SEQUENCE LISTING (22 sequences)

SEQ ID NO: 1 - DNA, 2151 bp, Bacillus thuringiensis
atgaattcatatcaaagtgaaaatgaatataaaatattggatacttcaaaaaatacctctactatgtctaatcgttatccaagatacccacaagcttctataaaaaatacaacttataaagattggttaaatatgtgtacaaaaaaccaatcaaatgctttgcaaattcctgaggctactcagatatcacaaggagcaatttctgctgcaatttctattagtacttcaattttaggtttgttaggtgttccatttgcatcacagatagggcaactttggcaatttacactcgagaagctatggcctgcagataatagtcaatgggaagaatttatgagacatgtagaagaactcattgatactaaaatagaaacatatgcaagagatacagcaattagtgaattaaaaggtttagttaatattttaagaccatatgaacgctcactcgaaaattggaaaaaacacccaacagacactggactgcaaaaaagcgtaagagacgcctttaaccttgttaatggtttttttaaagcaaccatgcctaaatttgctataagaaactatgaagtaccgttattaacagtgtatgcacttgcagccaatatgcatttacttttattacgagattcctctatttttgggagagaatgggggcttcctcaaaaggatgttgacgacaattactgtgatcaaagagcatttgcaaacgagtatgcaaatcattgtacaaattggtttaatagtggtttacaaagattaagaggaacaaatgcttctagttggatacggtataatagattccgtagagaaatgacgttgactgtattagatatctgctcattgttttccaattatgattattatcaatatccaattaaagtaaagtcagaacttaccagagaaatttatactgatccagtaggtgtggcaacgagcacgattcctggtctcattcctaactggtttgattatgcaccatcatttaatgagttagaacttgcactcattccgagtcctcgtacagttacttggttaaataaaataacaatttcaactggtagattaaacggctggagcacatcacagaactattggcaaggtcatagactagaatattcggaaacaagtgcaggtaataatataataagtccgacttatggggattttaacgaatcagttgctctccatagagattttagtgtattgaatagggatgtctacacaatggattcatatgcagtatcacaattttttggtattagtacagctaccctttttggagtttctgataatgatttccaataccaagaccttaatagtaatcggccaggggaaataagctatacaaatccacctagttggggtggccttcacatttattctgaattacctgcgaaaccaacaatacctgggatagaaccaggaaggccaactgctaacgattatagtcacagactaacgtatttgacagcttctcgtgcagggactgctggacatgttttatgttacggctggacatcttcaactgttgaccgtaacaatagaattgattacgataaaataacacaaataccggctgtgaaagggcattctcttcaaaatcatcgtcctcatgcatatacctatgtaataaaaggcaatcatacaggtgggaatttaatccgttttttaaaaacaagagaggcttataacgaatcaaccattggtggtggaattcgattgcatttttataattcaactgcgggacaaagttaccgcattcgttttcgatatgcggcagataaagctgcttattttagtgtatttcttcaaaaaagcggctggaattcaaatcagtttgtagagcttgaaaaaactttttctggaagttataacgatttaaattacaaagatttccaatacgctacactctcaattatcacacctccattacccggtggtgaaaatcagttatatctggacatagaggcgaatagttttcaatccgatgtaaatgtgattctcgacaaaattgaattcattccaagtaatatcccaattaaaaaatgtacatgtacatgcgacggaataaattgtgaattagaatgtacagaattacaatccgtagcaacagaaaaagagattgtaaatagtttatttaaaacaaagtag

SEQ ID NO: 2 - DNA, 1611 bp, Bacillus thuringiensis
atgtataccaatactatgaaaaatacattaaaactagaaacgacagattatgaaatagatcaagcggccatttctatagaatgtatgtcagatgaacaaaatccccaggaaaaaaagatgttatgggatgaagtaaaacatgccaaacaactcagtcaatctcgtaatttactctacaatggtgactttgaagatacatcaaacggctggaaaacaagctatacgattgaaattcgagagaatagtcccatttttaaagggcattatctgcatatgtttggggcaagagatattaatggaacgctatttccaacctatatctatcaaaaaatagatgaatcaaaattaaaaccctatacacgttatcgagtaagagggtttgtagaaagtagtaaagatttaacattagtggtaacacgttatgggaaagatattgatgcccacatggatgtcccaaatgatttgtcctatatgcagcctaacccttcatgtggagattatcgctgtgaatcatccccccagtatgtgagccaagggtattatacaccaacagatgtatatgctcctgataggtatgcatgcccgtcaagttcagataaaaaacatgttatgtgtcacgatcgtcatccctttgattttcatattgacaccggagaagtagatacgaatacaaacgtaggtattgatgtcttgtttaaaatttctaatccagatggatacgctacattagggaatctagaagtgattgaagaaggcccactaacagatgaagcattagcgcatgtgaagcaaaaggaaaagaaatggaaacaacacatggagaaaaaacgttgggaaacacaacaagcctatgacccagcaaaacaggcgatagatgcattatttacaaatgaacaagagttacactatcatattactttagatcatattcaaaacgccgatcagttgttacaggaaatcccctatgtataccatgattggttaccgaatgctccaggtatgaactatgatttatttaacaatttaaaggtacgtatagaacaagcacgctatttatatgatgcacgaaatgtcataacaaatggtgactttacacaggggttaacgggatggcacgcaacagggaaggcaacggtacaacagatgaatggcgcttctgtattagttctatcaaattggagtgcgggggtatctcaaaacttgcatgtccaagaccatcatggatatgtgctacgtgtgattgccaaaaaagaaggacctggaaaagggtatgtaacgatgatggattgtaatggaaatcaggaaacactgaagttcacttcttgtgaagaaggatatatgacaaaaacagtagaggtattcccagaaagtgatcgtgtacgaatagagatgggagaaaccgaaggtacgttttatatagatagcatcgagttgctttgtatgcaaggatatgctaccaataataactcgcactcgggtaatatgtatgagcaaagttataatgggaattataatcagaatatgagcgatgtgtatcaccaaggatatacaaacaactataaccaagggcataactctggctgtacatgtagtcaaggatataaacgttaa

SEQ ID NO: 3 - DNA, 3819 bp, Bacillus thuringiensis
atgaattcatatcaaagtgaaaatgaatataaaatattggatacttcaaaaaatacctctactatgtctaatcgttatccaagatacccacaagcttctataaaaaatacaacttataaagattggttaaatatgtgtacaaaaaaccaatcaaatgctttgcaaattcctgaggctactcagatatcacaaggagcaatttctgctgcaatttctattagtacttcaattttaggtttgttaggtgttccatttgcatcacagatagggcaactttggcaatttacactcgagaagctatggcctgcagataatagtcaatgggaagaatttatgagacatgtagaagaactcattgatactaaaatagaaacatatgcaagagatacagcaattagtgaattaaaaggtttagttaatattttaagaccatatgaacgctcactcgaaaattggaaaaaacacccaacagacactggactgcaaaaaagcgtaagagacgcctttaaccttgttaatggtttttttaaagcaaccatgcctaaatttgctataagaaactatgaagtaccgttattaacagtgtatgcacttgcagccaatatgcatttacttttattacgagattcctctatttttgggagagaatgggggcttcctcaaaaggatgttgacgacaattactgtgatcaaagagcatttgcaaacgagtatgcaaatcattgtacaaattggtttaatagtggtttacaaagattaagaggaacaaatgcttctagttggatacggtataatagattccgtagagaaatgacgttgactgtattagatatctgctcattgttttccaattatgattattatcaatatccaattaaagtaaagtcagaacttaccagagaaatttatactgatccagtaggtgtggcaacgagcacgattcctggtctcattcctaactggtttgattatgcaccatcatttaatgagttagaacttgcactcattccgagtcctcgtacagttacttggttaaataaaataacaatttcaactggtagattaaacggctggagcacatcacagaactattggcaaggtcatagactagaatattcggaaacaagtgcaggtaataatataataagtccgacttatggggattttaacgaatcagttgctctccatagagattttagtgtattgaatagggatgtctacacaatggattcatatgcagtatcacaattttttggtattagtacagctaccctttttggagtttctgataatgatttccaataccaagaccttaatagtaatcggccaggggaaataagctatacaaatccacctagttggggtggccttcacatttattctgaattacctgcgaaaccaacaatacctgggatagaaccaggaaggccaactgctaacgattatagtcacagactaacgtatttgacagcttctcgtgcagggactgctggacatgttttatgttacggctggacatcttcaactgttgaccgtaacaatagaattgattacgataaaataacacaaataccggctgtgaaagggcattctcttcaaaatcatcgtcctcatgcatatacctatgtaataaaaggcaatcatacaggtgggaatttaatccgttttttaaaaacaagagaggcttataacgaatcaaccattggtggtggaattcgattgcatttttataattcaactgcgggacaaagttaccgcattcgttttcgatatgcggcagataaagctgcttattttagtgtatttcttcaaaaaagcggctggaattcaaatcagtttgtagagcttgaaaaaactttttctggaagttataacgatttaaattacaaagatttccaatacgctacactctcaattatcacacctccattacccggtggtgaaaatcagttatatctggacatagaggcgaatagttttcaatccgatgtaaatgtgattctcgacaaaattgaattcattccaagtaatatcccaattaaaaaatgtacatgtacatgcgacggaataaattgtgaattagaatgtacagaattacaatccgtagcaacagaaaaagagattgtaaatagtttatttaaaacaaagtaggtaatgaagtaggaggtaaggatgctcgaaaaataagtagaaaaggtagtgaatcatatgtataccaatactatgaaaaatacattaaaactagaaacgacagattatgaaatagatcaagcggccatttctatagaatgtatgtcagatgaacaaaatccccaggaaaaaaagatgttatgggatgaagtaaaacatgccaaacaactcagtcaatctcgtaatttactctacaatggtgactttgaagatacatcaaacggctggaaaacaagctatacgattgaaattcgagagaatagtcccatttttaaagggcattatctgcatatgtttggggcaagagatattaatggaacgctatttccaacctatatctatcaaaaaatagatgaatcaaaattaaaaccctatacacgttatcgagtaagagggtttgtagaaagtagtaaagatttaacattagtggtaacacgttatgggaaagatattgatgcccacatggatgtcccaaatgatttgtcctatatgcagcctaacccttcatgtggagattatcgctgtgaatcatccccccagtatgtgagccaagggtattatacaccaacagatgtatatgctcctgataggtatgcatgcccgtcaagttcagataaaaaacatgttatgtgtcacgatcgtcatccctttgattttcatattgacaccggagaagtagatacgaatacaaacgtaggtattgatgtcttgtttaaaatttctaatccagatggatacgctacattagggaatctagaagtgattgaagaaggcccactaacagatgaagcattagcgcatgtgaagcaaaaggaaaagaaatggaaacaacacatggagaaaaaacgttgggaaacacaacaagcctatgacccagcaaaacaggcgatagatgcattatttacaaatgaacaagagttacactatcatattactttagatcatattcaaaacgccgatcagttgttacaggaaatcccctatgtataccatgattggttaccgaatgctccaggtatgaactatgatttatttaacaatttaaaggtacgtatagaacaagcacgctatttatatgatgcacgaaatgtcataacaaatggtgactttacacaggggttaacgggatggcacgcaacagggaaggcaacggtacaacagatgaatggcgcttctgtattagttctatcaaattggagtgcgggggtatctcaaaacttgcatgtccaagaccatcatggatatgtgctacgtgtgattgccaaaaaagaaggacctggaaaagggtatgtaacgatgatggattgtaatggaaatcaggaaacactgaagttcacttcttgtgaagaaggatatatgacaaaaacagtagaggtattcccagaaagtgatcgtgtacgaatagagatgggagaaaccgaaggtacgttttatatagatagcatcgagttgctttgtatgcaaggatatgctaccaataataactcgcactcgggtaatatgtatgagcaaagttataatgggaattataatcagaatatgagcgatgtgtatcaccaaggatatacaaacaactataaccaagggcataactctggctgtacatgtagtcaaggatataaacgttaa

SEQ ID NO: 4 - DNA, 2064 bp, Bacillus thuringiensis
atgtctaatcgttatccacggtatccaatagcaaataatccacaaatacctatgcgaaatacgaactataaagattggctaaatgagtgcgaaggaactaataatgaatgtcattcactcatatcgccatcaaatatagaatctgttatgcaaatttctttggaattagggttcgcactaataggagctctgggaggacctataggaagcataatatctgctattggaggtccagtagtaagtcttttatggggagaatttgggaatcaatgggatgcttcgagggatatggttgaagagcttataaatcaaacattagatgcaaatacaaggaatcaagctgcggccaaattagacagtttgaagaacatttttgatttatatataacgagattgaagacgtgggagaaaactcgtgatgctgatgatctaagggaattaacatcacagtttactacaactgataattttttaatagatagtttatcgttgtttaaacaagcaaatgttagacctatattgttaccgtcatatgtttatgcagcgactttacatttaagtctgttaagagattctattatatacgggaaggagtgggggtatagccaagagcttattgaatgtaattacgcaaggttacgagttgggattgcgaattattcggattattgtgcaacgacatatcatgagggcttaaacaatcttaggggttcaacttctagccagtggataaagtttaatagttaccgtacaaatttaacaataaatgcattagatttagtcatgttattcccattctatgatccgcgtttatatcctgaaaaagtaaatgcagagttaactagggaaatttatacagatccagtgggacatacgggttgggatcggaattttacaagttattttaatactctagaagctactggaacacggggacctggtttagttactaatctttcccataatatagatatatttagtgatgctttgataacgtacacaggcgcgactcctgtgaattatcttagcggctggggaggaactcgtcattatgaaaggtatacagggagttccgatattttgcaacgtatatctggaacaacgagtaatgatgtacatgattttaattcaatcagtcatacatttagaattaaatcagatgcaagggttgcggtaatagacgcgggtccgggtgtaggccctggaattcggcggtatcgtgtttcacgtgcagaatttcacgggactagtcgttttattgatgtgtataatgcaaatagtcctgttgggcagggcttaacgacatttgaatctatgttacccggttttcgttctcaagtacctaatcctctggattactctcatgaattatctaatgcggcatgtgtgcgatttgacagtgacagctccagaattaacgtatatggttggagacataaaagttcacagtttcgaaatgaaattcaagccaatcgaatttcacaaataccggcggtgaaaggttattaccttagcaattcttctacttatagctcccatgtaataaaaggcactaataccggtggggatttaatccattttttaaaaccaagagaggcttataacggagaaagtgcaggtggcggaattcgattgaatatttataattcaactgagggacaaagttaccgcattcgttttcgatatgcggcagataaagctgcttattttagtgtatttcttcaaaaaagcggctggaattcaaatcagtttgtagagcttgaaaaaacttactctggaaattataacgatttaaaatataatgatttccaatttgctgtactctcaattatcacacctccattacccggtggtgaaaatcagttatatctggacatggtagcgaatagttttcaatcagatgtaaatgtgattctcgacaaaattgaattcatcccaagtgatgtccaacttaaaaaatgtactgattgtcaatacaacccaagcgcaggcgtatgtgaatgtaaatgtgaaaaagtacaatccttggaaaaagagattgtaaatagtttatttgtcaaataa

SEQ ID NO: 5 - DNA, 1623 bp, Bacillus thuringiensis
atgtttacaagtggcacgaaaaatacgttgaaaatagaaacgacagattatgaaatagatcaagtggcaaattctatagaatgtatgtcagatgaacaaaatccacaggaaaaaacgatgttatgggatgaagtaaaacatgcaaaacaacttagtcagtctcgaaatttactccaaaatggtgactttggggacttagctggaaaggattggacattcaataatgatattatcataggatccaataatcctatttttaaagggaactttcttcagttacttggagcacgagacatatatggaagcatattcccaacttatatctatcaaaaaatagatgaatccaaattaaaaccatatacacgttatcgagtaagagggtttgtgggaagtagcaaagatttaacattagtggtaacacgttatgggaaagaaattgatgccattatggatgttccaaatgatttggcctatatgcagcctagcccttcatgtggagatgctcatcgctgtgaatcaccgtcccaagggtatcctacaccaacagatggatatgctcctgataggtatgcatgccagtccaatcaaggtaaaaagcatgtgaagtgtcacgatcgtcatccatttgatttccatattgacactggagaattagatataaatacaaacttaggtatctgtatcttatttaaaatttccaatccagatggatacgctacattagggaatttagaagtcattgaagaaggaccactaacaggcgaagcattggcacatgtgaaacataaggaaaagaaatggaatcaacacatggaaaaaaagcgaatggaaacacaagaagcctatgatccagcaaaactggcagtagatgcattatttacaaatgagcaagagttacaccatcatattactttagatcatattcaaaacgctgatcggctggtacagtcgattccatatgtacaccatgagtggttatcagatattccaggtatgaactatgatttatataccaatttaaaggtacgtatagtgcaagcacgctatttatatgatgcacgaaatgtcataaaaaatggtgactttacacaaggattactgggatggcacgcaactggaaaggtagcggtacaacaaatggatggcgcttctgtattagttctatcaaactggagtgccggggtatctcaaaatctgcatgcccaagatcatcatggatatgtgttacgtgtgattgccaaaaaagaaggacctggaaaagggtatgtaacgatgatggattgtaatggaaatcaggaaacactgaagttcacttcttgtgaagaaggatatatgacaaaaacagtagagatattcccggaaagcgatcgtgtacgaattgaaattggagaagccgaaggtacgttttatgtagaaagcatcgaattgatctgtatgaaaggttatactagcaattacaaccaaaatacgggtactatgtatgggcaaagttatactagcgattatagtcagaatacgagcaatatgtataatcaaggatataatagcaactataatcaaaatgataccaacaattatgaccagcatgccgattgttcgtgtaatcaaggatacagccgttaa

SEQ ID NO: 6 - DNA, 3754 bp, Bacillus thuringiensis
atgtctaatcgttatccacggtatccaatagcaaataatccacaaatacctatgcgaaatacgaactataaagattggctaaatgagtgcgaaggaactaataatgaatgtcattcactcatatcgccatcaaatatagaatctgttatgcaaatttctttggaattagggttcgcactaataggagctctgggaggacctataggaagcataatatctgctattggaggtccagtagtaagtcttttatggggagaatttgggaatcaatgggatgcttcgagggatatggttgaagagcttataaatcaaacattagatgcaaatacaaggaatcaagctgcggccaaattagacagtttgaagaacatttttgatttatatataacgagattgaagacgtgggagaaaactcgtgatgctgatgatctaagggaattaacatcacagtttactacaactgataattttttaatagatagtttatcgttgtttaaacaagcaaatgttagacctatattgttaccgtcatatgtttatgcagcgactttacatttaagtctgttaagagattctattatatacgggaaggagtgggggtatagccaagagcttattgaatgtaattacgcaaggttacgagttgggattgcgaattattcggattattgtgcaacgacatatcatgagggcttaaacaatcttaggggttcaacttctagccagtggataaagtttaatagttaccgtacaaatttaacaataaatgcattagatttagtcatgttattcccattctatgatccgcgtttatatcctgaaaaagtaaatgcagagttaactagggaaatttatacagatccagtgggacatacgggttgggatcggaattttacaagttattttaatactctagaagctactggaacacggggacctggtttagttactaatctttcccataatatagatatatttagtgatgctttgataacgtacacaggcgcgactcctgtgaattatcttagcggctggggaggaactcgtcattatgaaaggtatacagggagttccgatattttgcaacgtatatctggaacaacgagtaatgatgtacatgattttaattcaatcagtcatacatttagaattaaatcagatgcaagggttgcggtaatagacgcgggtccgggtgtaggccctggaattcggcggtatcgtgtttcacgtgcagaatttcacgggactagtcgttttattgatgtgtataatgcaaatagtcctgttgggcagggcttaacgacatttgaatctatgttacccggttttcgttctcaagtacctaatcctctggattactctcatgaattatctaatgcggcatgtgtgcgatttgacagtgacagctccagaattaacgtatatggttggagacataaaagttcacagtttcgaaatgaaattcaagccaatcgaatttcacaaataccggcggtgaaaggttattaccttagcaattcttctacttatagctcccatgtaataaaaggcactaataccggtggggatttaatccattttttaaaaccaagagaggcttataacggagaaagtgcaggtggcggaattcgattgaatatttataattcaactgagggacaaagttaccgcattcgttttcgatatgcggcagataaagctgcttattttagtgtatttcttcaaaaaagcggctggaattcaaatcagtttgtagagcttgaaaaaacttactctggaaattataacgatttaaaatataatgatttccaatttgctgtactctcaattatcacacctccattacccggtggtgaaaatcagttatatctggacatggtagcgaatagttttcaatcagatgtaaatgtgattctcgacaaaattgaattcatcccaagtgatgtccaacttaaaaaatgtactgattgtcaatacaacccaagcgcaggcgtatgtgaatgtaaatgtgaaaaagtacaatccttggaaaaagagattgtaaatagtttatttgtcaaataaaacaaaatatatactgaattaggtggtaaggctgttcgaaaaataagtagaaaaggtagtgaatcatatgtttacaagtggcacgaaaaatacgttgaaaatagaaacgacagattatgaaatagatcaagtggcaaattctatagaatgtatgtcagatgaacaaaatccacaggaaaaaacgatgttatgggatgaagtaaaacatgcaaaacaacttagtcagtctcgaaatttactccaaaatggtgactttggggacttagctggaaaggattggacattcaataatgatattatcataggatccaataatcctatttttaaagggaactttcttcagttacttggagcacgagacatatatggaagcatattcccaacttatatctatcaaaaaatagatgaatccaaattaaaaccatatacacgttatcgagtaagagggtttgtgggaagtagcaaagatttaacattagtggtaacacgttatgggaaagaaattgatgccattatggatgttccaaatgatttggcctatatgcagcctagcccttcatgtggagatgctcatcgctgtgaatcaccgtcccaagggtatcctacaccaacagatggatatgctcctgataggtatgcatgccagtccaatcaaggtaaaaagcatgtgaagtgtcacgatcgtcatccatttgatttccatattgacactggagaattagatataaatacaaacttaggtatctgtatcttatttaaaatttccaatccagatggatacgctacattagggaatttagaagtcattgaagaaggaccactaacaggcgaagcattggcacatgtgaaacataaggaaaagaaatggaatcaacacatggaaaaaaagcgaatggaaacacaagaagcctatgatccagcaaaactggcagtagatgcattatttacaaatgagcaagagttacaccatcatattactttagatcatattcaaaacgctgatcggctggtacagtcgattccatatgtacaccatgagtggttatcagatattccaggtatgaactatgatttatataccaatttaaaggtacgtatagtgcaagcacgctatttatatgatgcacgaaatgtcataaaaaatggtgactttacacaaggattactgggatggcacgcaactggaaaggtagcggtacaacaaatggatggcgcttctgtattagttctatcaaactggagtgccggggtatctcaaaatctgcatgcccaagatcatcatggatatgtgttacgtgtgattgccaaaaaagaaggacctggaaaagggtatgtaacgatgatggattgtaatggaaatcaggaaacactgaagttcacttcttgtgaagaaggatatatgacaaaaacagtagagatattcccggaaagcgatcgtgtacgaattgaaattggagaagccgaaggtacgttttatgtagaaagcatcgaattgatctgtatgaaaggttatactagcaattacaaccaaaatacgggtactatgtatgggcaaagttatactagcgattatagtcagaatacgagcaatatgtataatcaaggatataatagcaactataatcaaaatgataccaacaattatgaccagcatgccgattgttcgtgtaatcaaggatacagccgttaa

SEQ ID NO: 7 - protein, 716 aa, Bacillus thuringiensis
MNSYQSENEYKILDTSKNTSTMSNRYPRYPQASIKNTTYKDWLNMCTKNQSNALQIPEATQISQGAISAAISISTSILGLLGVPFASQIGQLWQFTLEKLWPADNSQWEEFMRHVEELIDTKIETYARDTAISELKGLVNILRPYERSLENWKKHPTDTGLQKSVRDAFNLVNGFFKATMPKFAIRNYEVPLLTVYALAANMHLLLLRDSSIFGREWGLPQKDVDDNYCDQRAFANEYANHCTNWFNSGLQRLRGTNASSWIRYNRFRREMTLTVLDICSLFSNYDYYQYPIKVKSELTREIYTDPVGVATSTIPGLIPNWFDYAPSFNELELALIPSPRTVTWLNKITISTGRLNGWSTSQNYWQGHRLEYSETSAGNNIISPTYGDFNESVALHRDFSVLNRDVYTMDSYAVSQFFGISTATLFGVSDNDFQYQDLNSNRPGEISYTNPPSWGGLHIYSELPAKPTIPGIEPGRPTANDYSHRLTYLTASRAGTAGHVLCYGWTSSTVDRNNRIDYDKITQIPAVKGHSLQNHRPHAYTYVIKGNHTGGNLIRFLKTREAYNESTIGGGIRLHFYNSTAGQSYRIRFRYAADKAAYFSVFLQKSGWNSNQFVELEKTFSGSYNDLNYKDFQYATLSIITPPLPGGENQLYLDIEANSFQSDVNVILDKIEFIPSNIPIKKCTCTCDGINCELECTELQSVATEKEIVNSLFKTK

SEQ ID NO: 8 - protein, 536 aa, Bacillus thuringiensis
MYTNTMKNTLKLETTDYEIDQAAISIECMSDEQNPQEKKMLWDEVKHAKQLSQSRNLLYNGDFEDTSNGWKTSYTIEIRENSPIFKGHYLHMFGARDINGTLFPTYIYQKIDESKLKPYTRYRVRGFVESSKDLTLVVTRYGKDIDAHMDVPNDLSYMQPNPSCGDYRCESSPQYVSQGYYTPTDVYAPDRYACPSSSDKKHVMCHDRHPFDFHIDTGEVDTNTNVGIDVLFKISNPDGYATLGNLEVIEEGPLTDEALAHVKQKEKKWKQHMEKKRWETQQAYDPAKQAIDALFTNEQELHYHITLDHIQNADQLLQEIPYVYHDWLPNAPGMNYDLFNNLKVRIEQARYLYDARNVITNGDFTQGLTGWHATGKATVQQMNGASVLVLSNWSAGVSQNLHVQDHHGYVLRVIAKKEGPGKGYVTMMDCNGNQETLKFTSCEEGYMTKTVEVFPESDRVRIEMGETEGTFYIDSIELLCMQGYATNNNSHSGNMYEQSYNGNYNQNMSDVYHQGYTNNYNQGHNSGCTCSQGYKR

SEQ ID NO: 9 - protein, 687 aa, Bacillus thuringiensis
MSNRYPRYPIANNPQIPMRNTNYKDWLNECEGTNNECHSLISPSNIESVMQISLELGFALIGALGGPIGSIISAIGGPVVSLLWGEFGNQWDASRDMVEELINQTLDANTRNQAAAKLDSLKNIFDLYITRLKTWEKTRDADDLRELTSQFTTTDNFLIDSLSLFKQANVRPILLPSYVYAATLHLSLLRDSIIYGKEWGYSQELIECNYARLRVGIANYSDYCATTYHEGLNNLRGSTSSQWIKFNSYRTNLTINALDLVMLFPFYDPRLYPEKVNAELTREIYTDPVGHTGWDRNFTSYFNTLEATGTRGPGLVTNLSHNIDIFSDALITYTGATPVNYLSGWGGTRHYERYTGSSDILQRISGTTSNDVHDFNSISHTFRIKSDARVAVIDAGPGVGPGIRRYRVSRAEFHGTSRFIDVYNANSPVGQGLTTFESMLPGFRSQVPNPLDYSHELSNAACVRFDSDSSRINVYGWRHKSSQFRNEIQANRISQIPAVKGYYLSNSSTYSSHVIKGTNTGGDLIHFLKPREAYNGESAGGGIRLNIYNSTEGQSYRIRFRYAADKAAYFSVFLQKSGWNSNQFVELEKTYSGNYNDLKYNDFQFAVLSIITPPLPGGENQLYLDMVANSFQSDVNVILDKIEFIPSDVQLKKCTDCQYNPSAGVCECKCEKVQSLEKEIVNSLFVK

SEQ ID NO: 10 - protein, 540 aa, Bacillus thuringiensis
MFTSGTKNTLKIETTDYEIDQVANSIECMSDEQNPQEKTMLWDEVKHAKQLSQSRNLLQNGDFGDLAGKDWTFNNDIIIGSNNPIFKGNFLQLLGARDIYGSIFPTYIYQKIDESKLKPYTRYRVRGFVGSSKDLTLVVTRYGKEIDAIMDVPNDLAYMQPSPSCGDAHRCESPSQGYPTPTDGYAPDRYACQSNQGKKHVKCHDRHPFDFHIDTGELDINTNLGICILFKISNPDGYATLGNLEVIEEGPLTGEALAHVKHKEKKWNQHMEKKRMETQEAYDPAKLAVDALFTNEQELHHHITLDHIQNADRLVQSIPYVHHEWLSDIPGMNYDLYTNLKVRIVQARYLYDARNVIKNGDFTQGLLGWHATGKVAVQQMDGASVLVLSNWSAGVSQNLHAQDHHGYVLRVIAKKEGPGKGYVTMMDCNGNQETLKFTSCEEGYMTKTVEIFPESDRVRIEIGEAEGTFYVESIELICMKGYTSNYNQNTGTMYGQSYTSDYSQNTSNMYNQGYNSNYNQNDTNNYDQHADCSCNQGYSR

SEQ ID NO: 11 - DNA, 31 bp, Artificial sequence
  Synthetic oligonucleotide
gccggatccaatgaattcatatcaaagtgaa

SEQ ID NO: 12 - DNA, 30 bp, Artificial sequence
  Synthetic oligonucleotide
ggggtcgacctactttgttttaaataaact

SEQ ID NO: 13 - DNA, 31 bp, Artificial sequence
  Synthetic oligonucleotide
gccggatccaatgtataccaatactatgaaa

SEQ ID NO: 14 - DNA, 31 bp, Artificial sequence
  Synthetic oligonucleotide
ggggtcgacttaacgtttatatccttgacta

SEQ ID NO: 15 - DNA, 21 bp, Artificial sequence
  Synthetic oligonucleotide
atgaattcatatcaaagtgaa

SEQ ID NO: 16 - DNA, 22 bp, Artificial sequence
  Synthetic oligonucleotide
ttaacgtttatatccttgacta

SEQ ID NO: 17 - DNA, 30 bp, Artificial sequence
  Synthetic oligonucleotide
ggggtcgacaatgtctaatcgttatccacg

SEQ ID NO: 18 - DNA, 30 bp, Artificial sequence
  Synthetic oligonucleotide
cccctcgagttatttgacaaataaactatt

SEQ ID NO: 19 - DNA, 31 bp, Artificial sequence
  Synthetic oligonucleotide
ggggtcgacaatgtttacaagtggcacgaaa

SEQ ID NO: 20 - DNA, 31 bp, Artificial sequence
  Synthetic oligonucleotide
cccctcgagttaacggctgtatccttgatta

SEQ ID NO: 21 - DNA, 20 bp, Artificial sequence
  Synthetic oligonucleotide
atgtctaatcgttatccacg

SEQ ID NO: 22 - DNA, 21 bp, Artificial sequence
  Synthetic oligonucleotide
ttaacggctgtatccttgatt

(19) United States
(12) Patent Application Publication
(10) Pub. No.: US 2016/0230187 A1
(43) Pub. Date: Aug. 11, 2016

(54) COMPOSITIONS AND METHODS FOR IMPROVING INSECT RESISTANCE

(71) Applicant: SICHUAN AGRICULTURAL UNIVERSITY, Chengdu (CN)

(72) Inventors: Aiping Zheng, Chengdu (CN); Ping Li, Chengdu (CN); Qiao Li, Chengdu (CN); Jun Zhu, Chengdu (CN); Qiming Deng, Chengdu (CN); Shiquan Wang, Chengdu (CN); Shuangcheng Li, Chengdu (CN)

(21) Appl. No.: 15/022,783

(22) PCT Filed: Sep. 17, 2014
(86) PCT No.: PCT/CN2014/086690

§ 371 Date: Mar. 17, 2016

(30) Foreign Application Priority Data
Sep. 18, 2013 (CN) 201310428864.9
Sep. 18, 2013 (CN) 201310428970.7
Sep. 18, 2013 (CN) 201310429403.3
Sep. 18, 2013 (CN) 201310430487.2

(51) Int. Cl. C12N 15/82; C07K 14/325

(52) U.S. Cl. CPC C12N 15/8286; C07K 14/325

(57) ABSTRACT

Compositions and methods for improving the pest resistance of plants. Plants and plant parts identified, selected and/or produced using the compositions and the methods.

### RELATED APPLICATIONS

This application claims priority to Chinese Provisional Patent Application Nos. 201310428864.9, filed Sep. 18, 2013; 201310428970.7, filed Sep. 18, 2013; 201310429403.3, filed Sep. 18, 2013; and 201310430487.2, filed Sep. 18, 2013, the disclosure of each of which is hereby incorporated by reference in its entirety.

### FIELD OF THE INVENTION

The present invention relates to compositions and methods for improving the pest resistances of plants.

### BACKGROUND OF THE INVENTION

Plant pests are a major factor in the loss of the world's important agricultural crops. About $8 billion are lost every year in the U.S. alone due to infestations of non-mammalian pests including insects. In addition to losses in field crops, insect pests are also a burden to vegetable and fruit growers, to producers of ornamental flowers, and to home gardeners.

Insect pests are mainly controlled by intensive applications of chemical pesticides, which inhibit insect growth, prevent insect feeding/reproduction, and/or cause insect death. Although chemical pesticides provide good insect pest control, they sometimes negatively affect other, beneficial insects. Another problem resulting from the wide use of chemical pesticides is the appearance of resistant insect varieties. This has been partially alleviated by various resistance management practices, but there is an increasing need for alternative pest control agents.

Biological pest control agents, such as Bacillus thuringiensis (Bt) strains expressing pesticidal toxins like δ-endotoxins, have also been applied to crop plants with satisfactory results, offering an alternative or compliment to chemical pesticides. The genes coding for some of those δ-endotoxins have been isolated and their expression in heterologous hosts have been shown to provide another tool for the control of economically important insect pests. In particular, the expression of insecticidal toxins in transgenic plants, such as Bt δ-endotoxins, has provided efficient protection against selected insect pests, and transgenic plants expressing such toxins have been commercialized, allowing farmers to reduce applications of chemical insect control agents.

The continued use of chemical and biological agents to control insect pests heightens the chance for insects to develop resistance to such control measures. Also, only a few specific insect pests are controllable with each control agent.

Thus, there remains a need to discover new and effective pest control agents that provide an economic benefit to farmers and that are environmentally acceptable. Particularly needed are new control agents that are targeted to a wide spectrum of economically important insect pests, new control agents that efficiently control insect strains that are or could become resistant to existing insect control agents, and new control agents with increased potency compared to current control agents. Furthermore, agents whose application minimizes burdens on the environment are desirable.

### SUMMARY OF THE INVENTION

The present invention addresses the need for new pest control agents by providing novel genes and toxins that may be used to control a variety of pests.

In particular, novel Cry nucleic acid sequences isolated from Bt, and sequences substantially identical thereto, whose expression results in pesticidal toxins with toxicity to economically important insect pests, particularly insect pests that infest plants, are provided. The invention is further drawn to the novel pesticidal toxins resulting from the expression of the nucleic acid sequences, and to compositions and formulations containing the pesticidal toxins, which are capable of inhibiting the ability of insect pests to survive, grow and reproduce, and of limiting insect-related damage or loss to crop plants. The invention is also drawn to methods of using the nucleic acid sequences, for example in making hybrid toxins with enhanced pesticidal activity or in a recombinogenic procedure such as DNA shuffling. The invention is further drawn to methods of making the toxins and to methods of using the nucleic acid sequences, for example in microorganisms to control insects or in transgenic plants to confer protection from insect damage, and to methods of using the pesticidal toxins, and compositions and formulations comprising the pesticidal toxins, for example applying the pesticidal toxins or compositions or formulations to insect-infested areas, or to prophylactically treat insect-susceptible areas or plants to confer protection against the insect pests.

The nucleotide sequences of the present invention can be engineered using methods generally known in the art in order to alter the nucleotide sequences for a variety of purposes including, but not limited to, broadening the spectrum of pesticidal activity, or increasing the specific activity against a specific pest. DNA shuffling by random fragmentation and PCR reassembly of gene fragments and synthetic oligonucleotides may be used to engineer the nucleotide sequences.

The novel pesticidal toxins described herein are highly active against insects. For example, a number of economically important insect pests, such as the lepidopterans Ostrinia nubilalis (European corn borer), Plutella xylostella (diamondback moth), Spodoptera frugiperda (fall armyworm), Agrotis ipsilon (black cutworm), Helicoverpa zea (corn earworm), Heliothis virescens (tobacco budworm), Spodoptera exigua (beet armyworm), Diatraea grandiosella (southwestern corn borer), Diatraea saccharalis (sugarcane borer), Helicoverpa punctigera (native budworm) and Helicoverpa armigera (cotton bollworm) can be controlled by the pesticidal toxins. The pesticidal toxins can be used singly or in combination with other insect control strategies to confer maximal pest control efficiency with minimal environmental impact.

In some embodiments, the present invention provides a nonnaturally occurring nucleic acid that encodes one or more δ-endotoxins and/or one or more auxiliary proteins capable of increasing the expression, stability and/or activity of one or more δ-endotoxins. For example, in some embodiments, the present invention provides nucleic acids comprising one or more of the nucleotide sequences set forth in SEQ ID NOs: 1-6, one or more nucleotide sequences that encodes a polypeptide comprising the amino acid sequence of any one of SEQ ID NOs: 7-10, one or more nucleotide sequences that is at least 95% identical to the nucleotide sequence set forth in any one of SEQ ID NOs: 1-6, one or more nucleotide sequences that encodes a polypeptide comprising an amino acid sequence that is at least 95% identical to the amino acid sequence of any one of SEQ ID NOs: 7-10, one or more nucleotide sequences that is complementary to one of the aforementioned nucleotide sequences, one or more nucleotide sequences that specifically hybridizes to one of the aforementioned nucleotide sequences under stringent hybridization conditions, and/or a functional fragment of one of the aforementioned nucleotide sequences.

In some embodiments, the present invention provides a transgenic bacterium, virus, fungal cell, plant or plant part that comprises an exogenous nucleic acid comprising one or more of the nucleotide sequences set forth in SEQ ID NOs: 1-6, one or more nucleotide sequences that encodes a polypeptide comprising the amino acid sequence of any one of SEQ ID NOs: 7-10, one or more nucleotide sequences that is at least 95% identical to the nucleotide sequence set forth in any one of SEQ ID NOs: 1-6, one or more nucleotide sequences that encodes a polypeptide comprising an amino acid sequence that is at least 95% identical to the amino acid sequence of any one of SEQ ID NOs: 7-10, one or more nucleotide sequences that is complementary to one of the aforementioned nucleotide sequences, one or more nucleotide sequences that specifically hybridizes to one of the aforementioned nucleotide sequences under stringent hybridization conditions, and/or a functional fragment of one of the aforementioned nucleotide sequences.

In some embodiments, the present invention provides a nonnaturally occurring δ-endotoxin. For example, in some embodiments, the present invention provides a pesticidal protein comprising an amino acid sequence that is at least 95% identical to the amino acid sequence of SEQ ID NO: 7 or SEQ ID NO: 9.

In some embodiments, the present invention provides a nonnaturally occurring δ-endotoxin chaperone. For example, in some embodiments, the present invention provides a chaperone comprising an amino acid sequence that is at least 95% identical to the amino acid sequence of SEQ ID NO: 8 or SEQ ID NO: 10.

In some embodiments, the present invention provides a pesticidal composition comprising a transgenic bacterium or fungus that expresses one or more of the nucleotide sequences set forth in SEQ ID NOs: 1-6, one or more nucleotide sequences that encodes a polypeptide comprising the amino acid sequence of any one of SEQ ID NOs: 7-10, one or more nucleotide sequences that is at least 95% identical to the nucleotide sequence set forth in any one of SEQ ID NOs: 1-6, one or more nucleotide sequences that encodes a polypeptide comprising an amino acid sequence that is at least 95% identical to the amino acid sequence of any one of SEQ ID NOs: 7-10, one or more nucleotide sequences that is complementary to one of the aforementioned nucleotide sequences, one or more nucleotide sequences that specifically hybridizes to one of the aforementioned nucleotide sequences under stringent hybridization conditions, and/or a functional fragment of one of the aforementioned nucleotide sequences.

In some embodiments, the present invention provides a pesticidal composition comprising a pesticidal protein having an amino acid sequence that is at least 95% identical to the amino acid sequence of SEQ ID NO: 7, a chaperone protein having an amino acid sequence that is at least 95% identical to the amino acid sequence of SEQ ID NO: 8, a pesticidal protein having an amino acid sequence that is at least 95% identical to the amino acid sequence of SEQ ID NO: 9, and/or a chaperone protein having an amino acid sequence that is at least 95% identical to the amino acid sequence of SEQ ID NO: 10.

In some embodiments, the present invention provides a method of identifying a plant or plant part having enhanced pest resistance, the method comprising detecting, in a plant or plant part, one or more nucleic acids that comprises one or more of the nucleotide sequences set forth in SEQ ID NOs: 1-6, one or more nucleotide sequences that encodes a polypeptide comprising the amino acid sequence of any one of SEQ ID NOs: 7-10, one or more nucleotide sequences that is at least 95% identical to the nucleotide sequence set forth in any one of SEQ ID NOs: 1-6, one or more nucleotide sequences that encodes a polypeptide comprising an amino acid sequence that is at least 95% identical to the amino acid sequence of any one of SEQ ID NOs: 7-10, one or more nucleotide sequences that is complementary to one of the aforementioned nucleotide sequences, one or more nucleotide sequences that specifically hybridizes to one of the aforementioned nucleotide sequences under stringent hybridization conditions, and/or a functional fragment of one of the aforementioned nucleotide sequences.

In some embodiments, the present invention provides a method of enhancing pest resistance in a plant or plant part, the method comprising expressing, in the plant or plant part, an exogenous nucleic acid comprising one or more of the nucleotide sequences set forth in SEQ ID NOs: 1-6, one or more nucleotide sequences that encodes a polypeptide comprising the amino acid sequence of any one of SEQ ID NOs: 7-10, one or more nucleotide sequences that is at least 95% identical to the nucleotide sequence set forth in any one of SEQ ID NOs: 1-6, one or more nucleotide sequences that encodes a polypeptide comprising an amino acid sequence that is at least 95% identical to the amino acid sequence of any one of SEQ ID NOs: 7-10, one or more nucleotide sequences that is complementary to one of the aforementioned nucleotide sequences, one or more nucleotide sequences that specifically hybridizes to one of the aforementioned nucleotide sequences under stringent hybridization conditions, and/or a functional fragment of one of the aforementioned nucleotide sequences. In some embodiments, the method further comprises introducing the exogenous nucleic acid into the plant or plant part.

In some embodiments, the present invention provides a method of producing a plant having enhanced pest resistance, the method comprising detecting, in a plant part, one or more nucleic acids comprising one or more of the nucleotide sequences set forth in SEQ ID NOs: 1-6, one or more nucleotide sequences that encodes a polypeptide comprising the amino acid sequence of any one of SEQ ID NOs: 7-10, one or more nucleotide sequences that is at least 95% identical to the nucleotide sequence set forth in any one of SEQ ID NOs: 1-6, one or more nucleotide sequences that encodes a polypeptide comprising an amino acid sequence that is at least 95% identical to the amino acid sequence of any one of SEQ ID NOs: 7-10, one or more nucleotide sequences that is complementary to one of the aforementioned nucleotide sequences, one or more nucleotide sequences that specifically hybridizes to one of the aforementioned nucleotide sequences under stringent hybridization conditions, and/or a functional fragment of one of the aforementioned nucleotide sequences; and producing a plant from the plant part.

In some embodiments, the present invention provides a method of producing a plant having enhanced pest resistance, the method comprising introducing, into a plant part, one or more nucleic acids comprising one or more of the nucleotide sequences set forth in SEQ ID NOs: 1-6, one or more nucleotide sequences that encodes a polypeptide comprising the amino acid sequence of any one of SEQ ID NOs: 7-10, one or more nucleotide sequences that is at least 95% identical to the nucleotide sequence set forth in any one of SEQ ID NOs: 1-6, one or more nucleotide sequences that encodes a polypeptide comprising an amino acid sequence that is at least 95% identical to the amino acid sequence of any one of SEQ ID NOs: 7-10, one or more nucleotide sequences that is complementary to one of the aforementioned nucleotide sequences, one or more nucleotide sequences that specifically hybridizes to one of the aforementioned nucleotide sequences under stringent hybridization conditions, and/or a functional fragment of one of the aforementioned nucleotide sequences; and producing a plant from the plant part.

In some embodiments, the present invention provides a method of producing a plant having enhanced pest resistance, the method comprising crossing a first parent plant or plant part with a second parent plant or plant part, wherein the first parent plant or plant part comprises within its genome one or more exogenous nucleic acids comprising one or more of the nucleotide sequences set forth in SEQ ID NOs: 1-6, one or more nucleotide sequences that encodes a polypeptide comprising the amino acid sequence of any one of SEQ ID NOs: 7-10, one or more nucleotide sequences that is at least 95% identical to the nucleotide sequence set forth in any one of SEQ ID NOs: 1-6, one or more nucleotide sequences that encodes a polypeptide comprising an amino acid sequence that is at least 95% identical to the amino acid sequence of any one of SEQ ID NOs: 7-10, one or more nucleotide sequences that is complementary to one of the aforementioned nucleotide sequences, one or more nucleotide sequences that specifically hybridizes to one of the aforementioned nucleotide sequences under stringent hybridization conditions, and/or a functional fragment of one of the aforementioned nucleotide sequences.

In some embodiments, the present invention provides a method of protecting a plant or plant part from a pest, the method comprising expressing, in the plant or plant part, an exogenous nucleic acid comprising one or more of the nucleotide sequences set forth in SEQ ID NOs: 1-6, one or more nucleotide sequences that encodes a polypeptide comprising the amino acid sequence of any one of SEQ ID NOs: 7-10, one or more nucleotide sequences that is at least 95% identical to the nucleotide sequence set forth in any one of SEQ ID NOs: 1-6, one or more nucleotide sequences that encodes a polypeptide comprising an amino acid sequence that is at least 95% identical to the amino acid sequence of any one of SEQ ID NOs: 7-10, one or more nucleotide sequences that is complementary to one of the aforementioned nucleotide sequences, one or more nucleotide sequences that specifically hybridizes to one of the aforementioned nucleotide sequences under stringent hybridization conditions, and/or a functional fragment of one of the aforementioned nucleotide sequences.

In some embodiments, the present invention provides a method of protecting a plant or plant part, the method comprising applying a pesticidal composition to the plant or plant part and/or to the area surrounding the plant or plant part, wherein the pesticidal composition comprises a pesticidal protein having an amino acid sequence that is at least 95% identical to the amino acid sequence of SEQ ID NO: 7, a chaperone protein having an amino acid sequence that is at least 95% identical to the amino acid sequence of SEQ ID NO: 8, a pesticidal protein having an amino acid sequence that is at least 95% identical to the amino acid sequence of SEQ ID NO: 9, and/or a chaperone protein having an amino acid sequence that is at least 95% identical to the amino acid sequence of SEQ ID NO: 10.

In some embodiments, the present invention provides a method of controlling a pest, the method comprising expressing, in the plant or plant part, an exogenous nucleic acid comprising one or more of the nucleotide sequences set forth in SEQ ID NOs: 1-6, one or more nucleotide sequences that encodes a polypeptide comprising the amino acid sequence of any one of SEQ ID NOs: 7-10, one or more nucleotide sequences that is at least 95% identical to the nucleotide sequence set forth in any one of SEQ ID NOs: 1-6, one or more nucleotide sequences that encodes a polypeptide comprising an amino acid sequence that is at least 95% identical to the amino acid sequence of any one of SEQ ID NOs: 7-10, one or more nucleotide sequences that is complementary to one of the aforementioned nucleotide sequences, one or more nucleotide sequences that specifically hybridizes to one of the aforementioned nucleotide sequences under stringent hybridization conditions, and/or a functional fragment of one of the aforementioned nucleotide sequences.

In some embodiments, the present invention provides a method of controlling a pest, the method comprising applying a pesticidal composition to the plant or plant part and/or to the area surrounding the plant or plant part, wherein the pesticidal compositions comprises a pesticidal protein having an amino acid sequence that is at least 95% identical to the amino acid sequence of SEQ ID NO: 7, a chaperone protein having an amino acid sequence that is at least 95% identical to the amino acid sequence of SEQ ID NO: 8, a pesticidal protein having an amino acid sequence that is at least 95% identical to the amino acid sequence of SEQ ID NO: 9, and/or a chaperone protein having an amino acid sequence that is at least 95% identical to the amino acid sequence of SEQ ID NO: 10.

In some embodiments, the present invention provides a method of controlling a pest, the method comprising applying a pesticidal composition to the pest and/or the pest's environment, wherein the pesticidal compositions comprises a pesticidal protein having an amino acid sequence that is at least 95% identical to the amino acid sequence of SEQ ID NO: 7, a chaperone protein having an amino acid sequence that is at least 95% identical to the amino acid sequence of SEQ ID NO: 8, a pesticidal protein having an amino acid sequence that is at least 95% identical to the amino acid sequence of SEQ ID NO: 9, and/or a chaperone protein having an amino acid sequence that is at least 95% identical to the amino acid sequence of SEQ ID NO: 10.

The foregoing and other objects and aspects of the present invention are explained in detail in the drawings and specification set forth below.

### BRIEF DESCRIPTION OF THE DRAWINGS

FIG. 1 shows an electrophoretogram of the full-length cry71Aa1 gene obtained by PCR amplification. Lane M is a DNA marker and lane 1 is the cry71Aa1 amplicon.

FIG. 2 shows the enzyme digestion products of the recombinant plasmid pS71Aa1. Lane M is a DNA marker, lane 1 is linearized pSTK plasmid, lane 2 is BamHI/SalI digestion products of the recombinant plasmid pS71Aa1, and lane 3 is the inserted DNA.

FIG. 3 shows the recombinant expression of Cry71Aa1 protein by no-crystal mutant strain HD73⁻ as determined by SDS-PAGE. Lane M is a protein marker; lane 1 is proteins expressed by transformants containing the recombinant plasmid pS71Aa1; and lane 2 is proteins expressed by the control transformant containing the plasmid pSTK. Cry71Aa1 protein is indicated with an arrow.

FIG. 4 shows the size of cry71Aa1 and cry71orf2 amplicons and BamHI+SalI-restricted plasmids containing said amplicons. Lane 1 is the cry71Aa1 amplicon, lane 2 is the recombinant plasmid pSTK-cry71Aa1 restricted with BamHI+SalI, lane 3 is the cry71orf2 amplicon, lane 4 is the recombinant plasmid pSTK-cry71orf2 restricted with BamHI+SalI, lane 5 is the cry71Aa1-cry71orf2 amplicon, lane 6 is the recombinant plasmid pSTK-cry71Aa1-cry71orf2 restricted with BamHI+SalI, lane 7 is linearized pSTK vector and M is the DNA marker.

FIG. 5 shows the recombinant expression of Cry71Aa1 and Cry71Orf2 proteins by no-crystal mutant strain HD73⁻ as determined by SDS-PAGE. Lane 1 is a crude protein extract of strain HS18-1, lane 2 is proteins expressed by transformants containing the recombinant plasmid pSTK-c71Aa1-cry71orf2, lane 3 is proteins expressed by transformants containing the recombinant plasmid pSTK-cry71Aa1, lane 4 is proteins expressed by transformants containing the recombinant plasmid pSTK-cry71orf2, lane 5 is proteins expressed by the control transformant containing the plasmid pSTK, and M is the protein marker. Cry71Aa1 and Cry71Orf2 proteins are indicated with arrows.

FIGS. 6A-6D show scanning electron microscopy images of the no-crystal mutant strain HD73⁻ transformed with pSTK (FIG. 6A) or no-crystal mutant strain HD73⁻ transformed with pSTK-cry71Aa1 (FIG. 6B); pSTK-cry71orf2 (FIG. 6C); or pSTK-cry71Aa1-cry71orf2 (FIG. 6D).

FIG. 7 shows the size of the cry72Aa1 amplicon and SalI+XhoI-restricted plasmid containing said amplicon. Lane 1 is the cry72Aa1 amplicon, lane 2 is the recombinant plasmid pSTK-cry72Aa1 restricted with SalI+XhoI, lane 3 is linearized pSTK vector and M is the DNA marker.

FIG. 8 shows the recombinant expression of Cry72Aa1 protein by no-crystal mutant strain HD73⁻ as determined by SDS-PAGE. Lane 1 is a crude protein extract of strain HS18-1, lane 2 is proteins expressed by transformants containing the recombinant plasmid pSTK-cry72Aa1, lane 3 is proteins expressed by the control transformant containing the plasmid pSTK, and M is the protein marker. Cry72Aa1 protein is indicated with an arrow.

FIGS. 9A-9C show the size of the cry72Aa1 and cry72orf2 amplicons and SalI+XhoI-restricted plasmids containing said amplicons. Lane 1 in FIG. 9A is the cry72Aa1 amplicon and lane 2 is the recombinant plasmid pSTK-cry72Aa1 restricted with SalI+XhoI. Lane 1 in FIG. 9B is the cry72orf2 amplicon and lane 2 is the recombinant plasmid pSTK-cry72orf2 restricted with SalI+XhoI. Lane 1 in FIG. 9C is the cry72Aa1-cry72orf2 amplicon and lane 2 is the recombinant plasmid pSTK-cry72Aa1-cry72orf2 restricted with SalI+XhoI. For each of FIGS. 9A-9C, lane 3 is linearized pSTK vector and M is the DNA marker.

FIG. 10 shows the recombinant expression of Cry72Aa1 and Orf2 proteins by no-crystal mutant strain HD73⁻ as determined by SDS-PAGE. Lane 1 is proteins expressed by transformants containing the recombinant plasmid pSTK-cry72Aa1-cry72orf2, lane 2 is proteins expressed by transformants containing the recombinant plasmid pSTK-cry72orf2, lane 3 is proteins expressed by transformants containing the recombinant plasmid pSTK-cry72Aa1, lane 4 is proteins expressed by the control transformant containing the plasmid pSTK, lane 5 is a crude protein extract of strain HS18-1 and M is the protein marker. Cry72Aa1 and Cry72Orf2 proteins are indicated with arrows.

FIGS. 11A-11D show the scanning electron microscopy images of the no-crystal mutant strain HD73⁻ transformed with pSTK (FIG. 11A) or no-crystal mutant strain HD73⁻ transformed with pSTK-cry72Aa1 (FIG. 11B); pSTK-cry72orf2 (FIG. 11C); or pSTK-cry72Aa1-cry72orf2 (FIG. 11D).

### DETAILED DESCRIPTION

The present invention provides compositions and methods for identifying, selecting and/or producing plants and plant parts having enhanced pest resistance (e.g., enhanced resistance to one or more Acarina and/or insects), as well as plants and plant parts identified, selected and/or produced using compositions and methods of the present invention.

Although the following terms are believed to be well understood by one of ordinary skill in the art, the following definitions are set forth to facilitate understanding of the presently disclosed subject matter.

All technical and scientific terms used herein, unless otherwise defined below, are intended to have the same meaning as commonly understood by one of ordinary skill in the art. References to techniques employed herein are intended to refer to the techniques as commonly understood in the art, including variations on those techniques or substitutions of equivalent techniques that would be apparent to one of skill in the art.

All patents, patent publications, non-patent publications referenced herein are incorporated by reference in their entireties for all purposes and to the same extent as if each was specifically and individually indicated to be incorporated by reference.

As used herein, the terms “a” or “an” or “the” may refer to one or more than one, unless the context clearly and unequivocally indicates otherwise. For example, “an” endogenous nucleic acid can mean one endogenous nucleic acid or a plurality of endogenous nucleic acids.

As used herein, the term “and/or” refers to and encompasses any and all possible combinations of one or more of the associated listed items, as well as the lack of combinations when interpreted in the alternative (“or”).

As used herein, the term “about,” when used in reference to a measurable value such as an amount of mass, dose, time, temperature, and the like, refers to a variation of 0.1%, 0.25%, 0.5%, 0.75%, 1%, 2%, 3%, 4%, 5%, 6,%, 7%, 8%, 9%, 10%, 15% or even 20% of the specified amount. Thus, if a given composition is described as comprising “about 50% X,” it is to be understood that, in some embodiments, the composition comprises 50% X whilst in other embodiments it may comprise anywhere from 40 to 60% X (i.e., 50±10%).

As used herein, the terms “abiotic stress” and “abiotic stress conditions” refer to non-living factors that negatively affect a plant's ability to grow, reproduce and/or survive (e.g., drought, flooding, extreme temperatures, extreme light conditions, extreme osmotic pressures, extreme salt concentrations, high winds, natural disasters and poor edaphic conditions (e.g., extreme soil pH, nutrient-deficient soil, compacted soil, etc.).

As used herein, the terms “abiotic stress tolerance” and “abiotic stress tolerant” refer to a plant's ability to endure and/or thrive under abiotic stress conditions (e.g., drought stress conditions, osmotic stress conditions, salt stress conditions and/or temperature stress conditions). When used in reference to a plant part, the terms refer to the ability of a plant that arises from that plant part to endure and/or thrive under abiotic stress conditions.

As used herein, the terms “backcross” and “backcrossing” refer to the process whereby a progeny plant is repeatedly crossed back to one of its parents. In a backcrossing scheme, the “donor” parent refers to the parental plant with the desired allele or locus to be introgressed. The “recipient” parent (used one or more times) or “recurrent” parent (used two or more times) refers to the parental plant into which the gene or locus is being introgressed. The initial cross gives rise to the F1 generation. The term “BC1” refers to the second use of the recurrent parent, “BC2” refers to the third use of the recurrent parent, and so on.

As used herein, the transitional phrase “consisting essentially of” is to be interpreted as encompassing the recited materials or steps and those that do not materially affect the basic and novel characteristic(s) of the claimed invention.

As used herein, the term “control” refers to the inhibition of an organism's ability to survive, grow, feed, and/or reproduce, and/or to limiting the damage/loss related to the activity of the organism. To “control” an organism may or may not mean killing the organism, although it preferably means killing the organism.

As used herein, the terms “cross,” “crossing” and “crossed” refer to the fusion of gametes to produce progeny (e.g., cells, seeds or plants). The term encompasses both sexual crosses (e.g., the pollination of one plant by another or the combination of protoplasts from two distinct plants via protoplast fusion) and selfing (e.g., self-pollination wherein the pollen and ovule are from the same plant).

As used herein, the term “CRY71 protein” refers to a δ-endotoxin having an amino acid sequence that is substantially identical to the amino acid sequence of Bt Cry71Aa1 or a functional fragment thereof. In some embodiments, the CRY71 protein has an amino acid sequence that is at least about 70%, 75%, 80%, 85%, 90%, 91%, 92%, 93%, 94%, 95%, 96%, 97%, 98%, 99%, 99.5% or more identical to the amino acid sequence of SEQ ID NO: 7 and/or to a functional fragment thereof In some embodiments, the CRY71 protein comprises an N-terminal helical bundle domain that is at least about 70%, 75%, 80%, 85%, 90%, 91%, 92%, 93%, 94%, 95%, 96%, 97%, 98%, 99%, 99.5% or more identical to amino acids 72 to 286 of SEQ ID NO: 7, a central beta-sheet domain that is at least about 70%, 75%, 80%, 85%, 90%, 91%, 92%, 93%, 94%, 95%, 96%, 97%, 98%, 99%, 99.5% or more identical to amino acids 295 to 511 of SEQ ID NO: 7, and/or a C-terminal beta-sandwich domain that is at least about 70%, 75%, 80%, 85%, 90%, 91%, 92%, 93%, 94%, 95%, 96%, 97%, 98%, 99%, 99.5% or more identical to amino acids 514 to 675 of SEQ ID NO: 7. In some embodiments, the CRY71 protein is encoded by a nucleic acid comprising a nucleotide sequence that is at least 70%, 75%, 80%, 85%, 90%, 91%, 92%, 93%, 94%, 95%, 96%, 97%, 98%, 99%, 99.5% or more identical to SEQ ID NO: 1 and/or to a functional fragment thereof.

As used herein, the term “CRY72 protein” refers to a δ-endotoxin having an amino acid sequence that is substantially identical to the amino acid sequence of Bt Cry72Aa1 or a functional fragment thereof In some embodiments, the CRY72 protein has an amino acid sequence that is at least about 70%, 75%, 80%, 85%, 90%, 91%, 92%, 93%, 94%, 95%, 96%, 97%, 98%, 99%, 99.5% or more identical to the amino acid sequence of SEQ ID NO: 9 and/or to a functional fragment thereof. In some embodiments, the CRY72 protein comprises an N-terminal helical bundle domain that is at least about 70%, 75%, 80%, 85%, 90%, 91%, 92%, 93%, 94%, 95%, 96%, 97%, 98%, 99%, 99.5% or more identical to amino acids 51 to 271 of SEQ ID NO: 9, a central beta-sheet domain that is at least about 70%, 75%, 80%, 85%, 90%, 91%, 92%, 93%, 94%, 95%, 96%, 97%, 98%, 99%, 99.5% or more identical to amino acids 279 to 481 of SEQ ID NO: 9, and/or a C-terminal beta-sandwich domain that is at least about 70%, 75%, 80%, 85%, 90%, 91%, 92%, 93%, 94%, 95%, 96%, 97%, 98%, 99%, 99.5% or more identical to amino acids 486 to 646 of SEQ ID NO: 9. In some embodiments, the CRY72 protein is encoded by a nucleic acid comprising a nucleotide sequence that is at least 70%, 75%, 80%, 85%, 90%, 91%, 92%, 93%, 94%, 95%, 96%, 97%, 98%, 99%, 99.5% or more identical to SEQ ID NO: 4 and/or to a functional fragment thereof.

As used herein, the terms “cultivar” and “variety” refer to a group of similar plants that by structural or genetic features and/or performance can be distinguished from other cultivars/varieties within the same species.

As used herein, the terms “decrease,” “decreases,” “decreasing” and similar terms refer to a reduction of at least about 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, 99.5% or more. In some embodiments, the reduction results in no or essentially no activity (i.e., an insignificant or undetectable amount of activity).

As used herein, the term “enhanced abiotic stress tolerance” refers to an improvement in the ability of a plant or plant part to grow, reproduce and/or survive under abiotic stress conditions, as compared to one or more controls (e.g., a native plant/plant part of the same species) “Enhanced ” may refer to any improvement in a plant's or plant part's ability to thrive and/or endure when grown under stress conditions, including, but not limited to, enhanced drought stress tolerance, osmotic stress tolerance, salt stress tolerance and/or temperature stress tolerance. In some embodiments, enhanced abiotic stress tolerance is evidenced by decreased water loss, decreased accumulation of one or more reactive oxygen species, decreased accumulation of one or more salts, increased salt excretion, increased accumulation of one or more dehydrins, improved root architecture, improved osmotic pressure regulation, increased accumulation of one or more late embryogenesis abundant proteins, increased survival rate, increased growth rate, increased height, increased chlorophyll content and/or increased yield (e.g., increased biomass, increased seed yield, increased grain yield at standard moisture percentage (YGSMN), increased grain moisture at harvest (GMSTP), increased grain weight per plot (GWTPN), increased percent yield recovery (PYREC), decreased yield reduction (YRED), and/or decreased percent barren (PB)) when grown under abiotic stress conditions. A plant or plant part that exhibits enhanced may be designated as “abiotic stress tolerant.”

As used herein, the term “enhanced pest resistance” refers to an improvement in the ability of a plant or plant part to grow, reproduce and/or survive under pest stress conditions, as compared to one or more controls (e.g., a native plant/plant part of the same species) “Enhanced pest resistance” may refer to any improvement in a plant's or plant part's ability to thrive and/or endure when grown under pest stress conditions, including, but not limited to, enhanced Acarina, bacterial, fungal, gastropod, insect, nematode, oomycete, phytoplasma, protozoa and/or viral resistance. In some embodiments, enhanced pest resistance is evidenced by increased survival rate, increased growth rate, increased height, and/or increased yield (e.g., increased biomass, increased seed yield, increased YGSMN, increased GMSTP, increased GWTPN, increased percent PYREC, and/or decreased YRED) when grown under pest stress conditions. A plant or plant part that exhibits enhanced pest resistance may be designated as “pest resistant.”

As used herein, the term “expression cassette” refers to a nucleic acid capable of directing expression of a particular nucleotide sequence in a host cell. The expression cassette may be chimeric, meaning that at least one of its components is heterologous with respect to at least one of its other components. The expression cassette may also be one that is naturally occurring but has been obtained in a recombinant form useful for heterologous expression. Typically, the expression cassette is heterologous with respect to the host (i.e., one or more of the nucleic acid sequences in the expression cassette do(es) not occur naturally in the host cell and must have been introduced into the host cell or an ancestor of the host cell by a transformation event).

As used herein, with respect to nucleic acids, the term “exogenous” refers to a nucleic acid that is not in the natural genetic background of the cell/organism in which it resides. In some embodiments, the exogenous nucleic acid comprises one or more nucleic acid sequences that are not found in the natural genetic background of the cell/organism. In some embodiments, the exogenous nucleic acid comprises one or more additional copies of a nucleic acid that is endogenous to the cell/organism.

As used herein with respect to nucleotide sequences, the terms “express” and “expression” refer to transcription and/or translation of the sequences.

As used herein with respect to nucleic acids, the term “fragment” refers to a nucleic acid that is reduced in length relative to a reference nucleic acid and that comprises, consists essentially of and/or consists of a nucleotide sequence of contiguous nucleotides identical or almost identical (e.g., 90%, 91%, 92%, 93%, 94%, 95%, 96%, 97%, 98%, 99% identical) to a corresponding portion of the reference nucleic acid. Such a nucleic acid fragment may be, where appropriate, included in a larger polynucleotide of which it is a constituent. In some embodiments, the nucleic acid fragment comprises, consists essentially of or consists of at least about 5, 6, 7, 8, 9, 10, 11, 12, 13, 14, 15, 20, 25, 30, 35, 40, 45, 50, 55, 60, 65, 70, 75, 80, 85, 90, 95, 100, 125, 150, 175, 200, 225, 250, 300, 350, 400, 450, 500, or more consecutive nucleotides. In some embodiments, the nucleic acid fragment comprises, consists essentially of or consists of less than about 5, 6, 7, 8, 9, 10, 11, 12, 13, 14, 15, 20, 25, 30, 35, 40, 45, 50, 55, 60, 65, 70, 75, 80, 85, 90, 95, 100, 125, 150, 175, 200, 225, 250, 300, 350, 400, 450 or 500 consecutive nucleotides.

As used herein with respect to polypeptides, the term “fragment” refers to a polypeptide that is reduced in length relative to a reference polypeptide and that comprises, consists essentially of and/or consists of an amino acid sequence of contiguous amino acids identical or almost identical (e.g., 90%, 91%, 92%, 93%, 94%, 95%, 96%, 97%, 98%, 99% identical) to a corresponding portion of the reference polypeptide. Such a polypeptide fragment may be, where appropriate, included in a larger polypeptide of which it is a constituent. In some embodiments, the polypeptide fragment comprises, consists essentially of or consists of at least about 2, 3, 4, 5, 6, 7, 8, 9, 10, 11, 12, 13, 14, 15, 20, 25, 30, 35, 40, 45, 50, 55, 60, 65, 70, 75, 80, 85, 90, 95, 100, 125, 150, 175, 200, 225, 250, 300, 350, 400, 450, 500, or more consecutive amino acids. In some embodiments, the polypeptide fragment comprises, consists essentially of or consists of less than about 2, 3, 4, 5, 6, 7, 8, 9, 10, 11, 12, 13, 14, 15, 20, 25, 30, 35, 40, 45, 50, 55, 60, 65, 70, 75, 80, 85, 90, 95, 100, 125, 150, 175, 200, 225, 250, 300, 350, 400, 450 or 500 consecutive amino acids.

As used herein with respect to nucleic acids, the term “functional fragment” refers to nucleic acid that encodes a functional fragment of a polypeptide.

As used herein with respect to polypeptides, the term “functional fragment” refers to polypeptide fragment that retains at least about 20%, 25%, 30%, 35%, 40%, 45%, 50%, 55%, 60%, 65%, 70%, 75%, 80%, 85%, 90%, 95%, 96%, 97%, 98%, 99%, 99.5% or more of at least one biological activity of the full-length polypeptide (e.g., insecticidal activity). In some embodiments, the functional fragment actually has a higher level of at least one biological activity of the full-length polypeptide.

As used herein, the term “germplasm” refers to genetic material of or from an individual plant, a group of plants (e.g., a plant line, variety or family), or a clone derived from a plant line, variety, species, or culture. The genetic material can be part of a cell, tissue or organism, or can be isolated from a cell, tissue or organism.

As used herein, the term “heterologous” refers to a nucleotide/polypeptide that originates from a foreign species, or, if from the same species, is substantially modified from its native form in composition and/or genomic locus by deliberate human intervention.

As used herein, the terms “increase,” “increases,” “increasing” and similar terms refer to an elevation of at least about 20%, 25%, 30%, 35%, 40%, 45%, 50%, 75%, 100%, 125%, 150%, 175%, 200%, 350%, 300%, 350%, 400%, 450%, 500% or more.

As used herein, the term “informative fragment” refers to a nucleotide sequence comprising a fragment of a larger nucleotide sequence, wherein the fragment allows for the identification of one or more alleles within the larger nucleotide sequence. For example, an informative fragment of the nucleotide sequence of SEQ ID NO: 1 comprises a fragment of the nucleotide sequence of SEQ ID NO: 1 and allows for the identification of one or more alleles located within the portion of the nucleotide sequence corresponding to that fragment of SEQ ID NO: 1.

As used herein with respect to nucleic acids, nucleotides and polypeptides, the term “isolated” refers to a nucleic acid, nucleotide or polypeptide that, by the hand of man, exists apart from its native environment and is therefore not a product of nature. In some embodiments, the nucleic acid, nucleotide or polypeptide exists in a purified form that is substantially free of cellular material, viral material, culture medium (when produced by recombinant DNA techniques), or chemical precursors or other chemicals (when chemically synthesized). An “isolated fragment” is a fragment of a nucleotide or polypeptide that is not naturally occurring as a fragment and would not be found in the natural state. “Isolated” does not mean that the preparation is technically pure (homogeneous), but rather that it is sufficiently pure to provide the nucleotide or polypeptide in a form in which it can be used for the intended purpose. In certain embodiments, the composition comprising the nucleotide or polypeptide is at least about 50%, 55%, 60%, 65%, 70%, 75%, 80%, 85%, 90%, 95%, 96%, 97%, 98%, or 99% or more pure.

As used herein with respect to cells, the term “isolated” refers to a cell that, by the hand of man, exists apart from its native environment and is therefore not a product of nature. In some embodiments, the cell is separated from other components with which it is normally associated in its natural state. For example, an isolated plant cell may be a plant cell in culture medium and/or a plant cell in a suitable carrier. “Isolated” does not mean that the preparation is technically pure (homogeneous), but rather that it is sufficiently pure to provide the cell in a form in which it can be used for the intended purpose. In certain embodiments, the composition comprising the cell is at least about 50%, 55%, 60%, 65%, 70%, 75%, 80%, 85%, 90%, 95%, 96%, 97%, 98%, or 99% or more pure.

As used herein with respect to nucleic acids, the term “nonfunctional fragment” refers to nucleic acid that encodes a nonfunctional fragment of a polypeptide.

As used herein with respect to polypeptides, the term “nonfunctional fragment” refers to polypeptide fragment that exhibits none or essentially none (i.e., less than about 10%, 9%, 8%, 7%, 6%, 5%, 4%, 3%, 2%, 1% or less) of the biological activities of the full-length polypeptide.

As used herein with respect to nucleic acids, proteins, plants, plant parts, bacteria, viruses and fungi, the term “nonnaturally occurring” refers to nucleic acids, proteins, plants, plant parts, bacteria, viruses or fungi that do not naturally exist in nature. Nonnaturally occurring nucleic acids, proteins, plants, plant parts, bacteria, viruses and fungi of the present invention may comprise any suitable variation(s) from their closest naturally occurring counterparts. For example, nonnaturally occurring nucleic acids of the present invention may comprise an otherwise naturally occurring nucleotide sequence having one or more point mutations, insertions or deletions relative to the naturally occurring nucleotide sequence. In some embodiments, nonnaturally occurring nucleic acids of the present invention comprise a naturally occurring nucleotide sequence and one or more heterologous nucleotide sequences (e.g., one or more heterologous promoter sequences, intron sequences and/or termination sequences). Likewise, nonnaturally occurring proteins of the present invention may comprise an otherwise naturally occurring protein that comprises one or more mutations, insertions, additions or deletions relative to the naturally occurring protein (e.g., one or more epitope tags). Similarly, nonnaturally occurring plants, plant parts, bacteria, viruses and fungi of the present invention may comprise one more exogenous nucleotide sequences and/or one or more nonnaturally occurring copies of a naturally occurring nucleotide sequence (i.e., extraneous copies of a gene that naturally occurs in that species). Nonnaturally occurring plants and plant parts may be produced by any suitable method, including, but not limited to, transfecting/transducing a plant or plant part with an exogenous nucleic acid and crossing a naturally occurring plant or plant part with a nonnaturally occurring plant or plant part. It is to be understood that all nucleic acids, proteins, plants, plant parts, bacteria, viruses and fungi claimed herein are nonnaturally occurring.

As used herein, the term “nucleic acid” refers to deoxyribonucleotide, ribonucleotide and deoxyribonucleotide-ribonucleotide polymers in either single- or double-stranded form and, unless otherwise limited, encompasses analogues having the essential nature of natural nucleotide sequences in that they hybridize to single-stranded nucleic acids in a manner similar to naturally occurring nucleotides (e.g., peptide nucleic acids).

As used herein, the term “nucleotide” refers to a monomeric unit from which DNA or RNA polymers are constructed and which consists of a purine or pyrimidine base, a pentose, and a phosphoric acid group. Nucleotides (usually found in their 5′-monophosphate form) are referred to by their single letter designation as follows: “A” for adenylate or deoxyadenylate (for RNA or DNA, respectively), “C” for cytidylate or deoxycytidylate, “G” for guanylate or deoxyguanylate, “U” for uridylate, “T” for deoxythymidylate, “R” for purines (A or G), “Y” for pyrimidines (C or T), “K” for G or T, “H” for A or C or T, “I” for inosine, and “N” for any nucleotide.

As used herein, the terms “nucleotide sequence,” “polynucleotide,” “nucleic acid sequence,” “nucleic acid molecule” and “nucleic acid fragment” refer to a polymer of RNA, DNA, or RNA and DNA that is single- or double-stranded, optionally containing synthetic, non-natural and/or altered nucleotide bases.

As used herein, the term “nucleotide sequence identity” refers to the presence of identical nucleotides at corresponding positions of two polynucleotides. Polynucleotides have “identical” sequences if the sequence of nucleotides in the two polynucleotides is the same when aligned for maximum correspondence (e.g., in a comparison window). Sequence comparison between two or more polynucleotides is generally performed by comparing portions of the two sequences over a comparison window to identify and compare local regions of sequence similarity. The comparison window is generally from about 20 to 200 contiguous nucleotides. The “percentage of sequence identity” for polynucleotides, such as about 50, 55, 60, 65, 70, 75, 80, 85, 90, 95, 98, 99 or 100 percent sequence identity, can be determined by comparing two optimally aligned sequences over a comparison window, wherein the portion of the polynucleotide sequence in the comparison window can include additions or deletions (i.e., gaps) as compared to the reference sequence for optimal alignment of the two sequences. The percentage is calculated by: (a) determining the number of positions at which the identical nucleic acid base occurs in both sequences; (b) dividing the number of matched positions by the total number of positions in the window of comparison; and (c) multiplying the result by 100. Optimal alignment of sequences for comparison can also be conducted by computerized implementations of known algorithms, or by visual inspection. Readily available sequence comparison and multiple sequence alignment algorithms are, respectively, the Basic Local Alignment Search Tool (BLAST) and ClustalW programs, both available on the internet. Other suitable programs include, but are not limited to, GAP, BestFit, Plot Similarity, and FASTA, which are part of the Accelrys GCG Package available from Accelrys, Inc. of San Diego, Calif., United States of America. In some embodiments, a percentage of sequence identity refers to sequence identity over the full length of one of the sequences being compared. In some embodiments, a calculation to determine a percentage of sequence identity does not include in the calculation any nucleotide positions in which either of the compared nucleic acids includes an “N” (i.e., where any nucleotide could be present at that position).

As used herein with respect to nucleic acids, the term “operably linked” refers to a functional linkage between two or more nucleic acids. For example, a promoter sequence may be described as being “operably linked” to a heterologous nucleic acid sequence because the promoter sequences initiates and/or mediates transcription of the heterologous nucleic acid sequence. In some embodiments, the operably linked nucleic acid sequences are contiguous and/or are in the same reading frame.

As used herein, the term “ORF2 protein” refers to a protein having an amino acid sequence that is substantially identical to the amino acid sequence of Bt Cry71Orf2, Bt Cry72Orf2 or a functional fragment thereof In some embodiments, the ORF2 protein has an amino acid sequence that is at least about 70%, 75%, 80%, 85%, 90%, 91%, 92%, 93%, 94%, 95%, 96%, 97%, 98%, 99%, 99.5% or more identical to the amino acid sequence of SEQ ID NO: 8, SEQ ID NO: 10, and/or to a functional fragment thereof.

As used herein, the term “percent yield recovery” (PYREC) refers to the effect a nucleotide sequence and/or combination of nucleotide sequences has on the yield of a plant grown under stress conditions (e.g., pest stress conditions) as compared to that of a control plant that is genetically identical except insofar as it lacks the nucleotide sequence and/or combination of nucleotide sequences. PYREC is calculated as:

$$1 - \frac{\begin{matrix}\begin{matrix}yield under non \text{-} stress \\ ( w \text{/} nucleotide sequence ( s ) of interest ) - \end{matrix} \\ yield under stres s conditions \\ ( w \text{/} nucleotide sequence ( s ) of interest )\end{matrix}}{\begin{matrix}\begin{matrix}yield under non \text{-} stress \\ ( w \text{/} out nucleotide sequence ( s ) of interest ) - \end{matrix} \\ yield under stress conditions \\ ( w \text{/} out nucleotide sequence ( s ) of interest )\end{matrix}} \times 100$$

By way of example and not limitation, if a control plant yields 200 bushels under full irrigation conditions, but yields only 100 bushels under pest stress conditions, then its percentage yield loss would be calculated at 50%. If an otherwise genetically identical hybrid that contains the nucleotide sequence(s) of interest yields 125 bushels under pest stress conditions and 200 bushels under full irrigation conditions, then the percentage yield loss would be calculated as 37.5% and the PYREC would be calculated as 25% [1.00−(200−125)/(200−100)×100)].

As used herein, the terms “pest resistance” and “pest tolerant” refer to a plant's ability to endure and/or thrive under pest stress conditions. When used in reference to a plant part, the terms refer to the ability of a plant that arises from that plant part to endure and/or thrive under pest stress conditions.

As used herein, the terms “pest stress” and “pest stress conditions” refer to stress(es) caused by organisms that negatively affect a plant's ability to grow, reproduce and/or survive (e.g., Acarina, bacteria, fungi, gastropods, insects, nematodes, oomycetes, phytoplasma, protozoa and/or viruses). In some embodiments, “pest stress conditions” comprise infestation by one or more pests (e.g., one or more Acarina and/or insect pests).

As used herein, the term “pesticidal” refers to the ability of a molecule/compound to control one or more pests. Thus, a pesticidal Cry71 protein may inhibit the ability of a pest organism (e.g., an insect pest) to survive, grow, feed, and/or reproduce. In some embodiments, the pesticidal molecule/compound kills the pest.

As used herein, the term “pesticidally effective amount” refers to a concentration or amount that inhibits, through a toxic effect, the ability of one or more pests to survive, grow, feed and/or reproduce, and/or that limits pest-related damage or loss in crop plants. A “pesticidally effective amount,” may or may not kill the pest(s), although it preferably kills the pest(s).

As used herein, the terms “phenotype,” “phenotypic trait” or “trait” refer to one or more traits of an organism. The phenotype can be observable to the naked eye, or by any other means of evaluation known in the art, e.g., microscopy, biochemical analysis, or an electromechanical assay. In some cases, a phenotype is directly controlled by a single gene or genetic locus, i.e., a “single gene trait.” In other cases, a phenotype is the result of several genes. It is noted that, as used herein, the term “water optimization phenotype” takes into account environmental conditions that might affect water optimization such that the water optimization effect is real and reproducible.

As used herein, the term “plant” may refer to any suitable plant, including, but not limited to, spermatophytes (e.g., angiosperms and gymnosperms) and embryophytes (e.g., bryophytes, ferns and fern allies). In some embodiments, the plant is a monocotyledonous (monocot) plant such as a rice, maize, wheat, barley, sorghum, millet, oat, triticale, rye, buckwheat, fonio, quinoa, sugar cane, bamboo, banana, ginger, onion, lily, daffodil, iris, amaryllis, orchid, canna, bluebell, tulip, garlic, secale, einkorn, spelt, emmer, durum, kamut, grass (e.g., gramma grass), teff, milo, flax, Tripsacum sp., or teosinte plant. In some embodiments, the plant is a dicotyledonous (dicot) plant such as a blackberry, raspberry, strawberry, barberry, bearberry, blueberry, coffee berry, cranberry, crowberry, currant, elderberry, gooseberry, goji berry, honeyberry, lemon, lime, lingonberry, mangosteen, orange, pepper, persimmon, pomegranate, prune, cotton, clover, acai, plum, peach, nectarine, cherry, guava, almond, pecan, walnut, amaranth, apple, sweet pea, pear, potato, soybean, sugar beet, sunflower, sweet potato, tamarind, tea, tobacco or tomato plant.

As used herein, the term “plant cell” refers to a cell existing in, taken from and/or derived from a plant (e.g., a cell derived from a plant cell/tissue culture). Thus, the term “plant cell” may refer to an isolated plant cell, a plant cell in a culture, a plant cell in an isolated tissue/organ and/or a plant cell in a whole plant.

As used herein, the term “plant part” refers to at least a fragment of a whole plant or to a cell culture or tissue culture derived from a plant. Thus, the term “plant part” may refer to a plant cell, a plant tissue and/or a plant organ, as well as to a cell/tissue culture derived from a plant cell, plant tissue or plant culture. Embodiments of the present invention may comprise and/or make use of any suitable plant part, including, but not limited to, anthers, branches, buds, calli, clumps, cobs, cotyledons, ears, embryos, filaments, flowers, fruits, husks, kernels, leaves, lodicules, ovaries, palea, panicles, pedicels, pods, pollen, protoplasts, roots, root tips, seeds, silks, stalks, stems, stigma, styles, and tassels. In some embodiments, the plant part is a plant germplasm.

As used herein, the term “polynucleotide” refers to a deoxyribopolynucleotide, ribopolynucleotide or analogs thereof that have the essential nature of a natural deoxyribopolynucleotide/ribonucleotide in that they hybridize, under stringent hybridization conditions, to substantially the same nucleotide sequence as naturally occurring nucleotides and/or allow translation into the same amino acid(s) as the naturally occurring nucleotide(s). A polynucleotide can be full-length or a subsequence of a native or heterologous structural or regulatory gene. Unless otherwise indicated, the term includes reference to the specified sequence as well as the complementary sequence thereof Thus, DNAs or RNAs with backbones modified for stability or for other reasons are “polynucleotides” as that term is intended herein. Moreover, DNAs or RNAs comprising unusual bases, such as inosine or modified bases, such as tritylated bases, to name just two examples, are polynucleotides as the term is used herein. It will be appreciated that a great variety of modifications have been made to DNA and RNA that serve many useful purposes known to those of skill in the art. The term polynucleotide as it is employed herein embraces such chemically, enzymatically or metabolically modified forms of polynucleotides, as well as the chemical forms of DNA and RNA characteristic of viruses and cells, including inter alia, simple and complex cells.

As used herein, the terms “polypeptide,” “peptide” and “protein” refer to a polymer of amino acid residues. The terms encompass amino acid polymers in which one or more amino acid residue is an artificial chemical analogue of a corresponding naturally occurring amino acid, as well as to naturally occurring amino acid polymers.

As used herein, the terms “progeny” and “progeny plant” refer to a plant generated from a vegetative or sexual reproduction from one or more parent plants. A progeny plant may be obtained by cloning or selfing a single parent plant, or by crossing two parental plants.

As used herein, the terms “promoter” and “promoter sequence” refer to nucleic acid sequences involved in the regulation of transcription initiation. A “plant promoter” is a promoter capable of initiating transcription in plant cells. Exemplary plant promoters include, but are not limited to, those that are obtained from plants, from plant viruses and from bacteria that comprise genes expressed in plant cells such Agrobacterium or Rhizobium. A “tissue-specific promoter” is a promoter that preferentially initiates transcription in a certain tissue (or combination of tissues). A “stress-inducible promoter” is a promoter that preferentially initiates transcription under certain environmental conditions (or combination of environmental conditions). A “developmental stage-specific promoter” is a promoter that preferentially initiates transcription during certain developmental stages (or combination of developmental stages).

As used herein, the term “regulatory sequences” refers to nucleotide sequences located upstream (5′ non-coding sequences), within or downstream (3′ non-coding sequences) of a coding sequence, which influence the transcription, RNA processing or stability, or translation of the associated coding sequence. Regulatory sequences include, but are not limited to, promoters, enhancers, exons, introns, translation leader sequences, termination signals, and polyadenylation signal sequences. Regulatory sequences include natural and synthetic sequences as well as sequences that can be a combination of synthetic and natural sequences. An “enhancer” is a nucleotide sequence that can stimulate promoter activity and can be an innate element of the promoter or a heterologous element inserted to enhance the level or tissue specificity of a promoter. The coding sequence can be present on either strand of a double-stranded DNA molecule, and is capable of functioning even when placed either upstream or downstream from the promoter.

As used herein, the terms “selectively hybridize” and “specifically hybridize” refer to the hybridization of a nucleic acid sequence to a specified nucleic acid target sequence, wherein the nucleic acid sequence preferentially hybridizes to the specified nucleic acid target sequence to the substantial exclusion of non-target nucleic acids (e.g., at least about a two- to ten-fold difference as compared to its hybridization with non-target nucleic acid sequences).

As used herein, the terms “stringent hybridization conditions” and “stringent hybridization wash conditions” refer to conditions under which a nucleic acid will selectively hybridize to a target nucleic acid sequence. In some embodiments, stringent hybridization conditions comprise 7% sodium dodecyl sulfate (SDS), 0.5 M Na₃PO₄, 1 mM EDTA at 50° C. with washing in 2×SSC, 0.1% SDS at 50° C. In some embodiments, stringent hybridization conditions comprise 7% SDS, 0.5 M Na₃PO₄, 1 mM EDTA at 50° C. with washing in 1×SSC, 0.1% SDS at 50° C. In some embodiments, stringent hybridization conditions comprise 7% SDS, 0.5 M Na₃PO₄, 1 mM EDTA at 50° C. with washing in 0.5×SSC, 0.1% SDS at 50° C. In some embodiments, stringent hybridization conditions comprise 7% SDS, 0.5 M Na₃PO₄, 1 mM EDTA at 50° C. with washing in 0.1×SSC, 0.1% SDS at 50° C. In some embodiments, stringent hybridization conditions comprise 7% SDS, 0.5 M Na₃PO₄, 1 mM EDTA at 50° C. with washing in 0.1×SSC, 0.1% SDS at 65° C. In some embodiments, stringent hybridization conditions comprise 6×SSC, 0.5% SDS at 65° C. with washing in 2×SSC, 0.1% SDS and 1×SSC, 0.1% SDS at 65° C. In some embodiments, stringent hybridization conditions comprise a wash stringency of 50% formamide with 5× Denhardt's solution, 0.5% SDS and 1× SSPE at 42° C.

“Stringent hybridization conditions” and “stringent hybridization wash conditions” in the context of nucleic acid hybridization experiments such as Southern and Northern hybridizations are sequence dependent, and are different under different environmental parameters. An extensive guide to the hybridization of nucleic acids is found in Tijssen Laboratory Techniques in Biochemistry and Molecular Biology-Hybridization with Nucleic Acid Probes part I chapter 2 “Overview of principles of hybridization and the strategy of nucleic acid probe assays” Elsevier, New York (1993). Generally, highly stringent hybridization and wash conditions are selected to be about 5° C. lower than the thermal melting point (Tₘ) for the specific sequence at a defined ionic strength and pH.

The Tₘ is the temperature (under defined ionic strength and pH) at which 50% of the target sequence hybridizes to a perfectly matched probe. Very stringent conditions are selected to be equal to the Tₘ for a particular probe. An example of stringent hybridization conditions for hybridization of complementary nucleotide sequences which have more than 100 complementary residues on a filter in a Southern or northern blot is 50% formamide with 1 mg of heparin at 42° C., with the hybridization being carried out overnight. An example of highly stringent wash conditions is 0.1 5M NaCl at 72° C. for about 15 minutes. An example of stringent wash conditions is a 0.2×SSC wash at 65° C. for 15 minutes (see, Sambrook, infra, for a description of SSC buffer). Often, a high stringency wash is preceded by a low stringency wash to remove background probe signal. An example of a medium stringency wash for a duplex of, e.g., more than 100 nucleotides, is 1×SSC at 45° C. for 15 minutes. An example of a low stringency wash for a duplex of, e.g., more than 100 nucleotides, is 4-6×SSC at 40° C. for 15 minutes. For short probes (e.g., about 10 to 50 nucleotides), stringent conditions typically involve salt concentrations of less than about 1.0 M Na ion, typically about 0.01 to 1.0 M Na ion concentration (or other salts) at pH 7.0 to 8.3, and the temperature is typically at least about 30° C. Stringent conditions can also be achieved with the addition of destabilizing agents such as formamide. In general, a signal to noise ratio of 2× (or higher) than that observed for an unrelated probe in the particular hybridization assay indicates detection of a specific hybridization. Nucleotide sequences that do not hybridize to each other under stringent conditions may still be substantially identical if the proteins that they encode are substantially identical. This can occur, for example, when a copy of a nucleotide sequence is created using the maximum codon degeneracy permitted by the genetic code.

As used herein, the term “substantially identical,” in the context of two nucleic acid molecules, nucleotide sequences or protein sequences, refers to two or more sequences or subsequences that have at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, and/or 100% nucleotide or amino acid residue identity, when compared and aligned for maximum correspondence, as measured using one of the following sequence comparison algorithms or by visual inspection. In some embodiments of the invention, the substantial identity exists over a region of the sequences that is at least about 50 residues to about 750 residues in length. Thus, in some embodiments, substantial identity exists over a region of the sequences that is at least about 50 residues to about 250 residues in length, about 75 residues to about 225 residues in length, about 100 residues to about 200 residues in length, about 125 residues to about 175 residues in length, about 200 residues to about 400 residues in length, about 300 residues to about 450 residues in length, about 400 residues to about 500 residues in length, about 500 residues to about 550 residues in length, about 550 residues to about 650 residues in length, and/or about 650 residues to about 750 residues in length, or any value or range therein. For sequence comparison, typically one sequence acts as a reference sequence to which test sequences are compared. When using a sequence comparison algorithm, test and reference sequences are entered into a computer, subsequence coordinates are designated if necessary, and sequence algorithm program parameters are designated. The sequence comparison algorithm then calculates the percent sequence identity for the test sequence(s) relative to the reference sequence, based on the designated program parameters.

Optimal alignment of sequences for aligning a comparison window are well known to those skilled in the art and may be conducted by tools such as the local homology algorithm of Smith and Waterman, the homology alignment algorithm of Needleman and Wunsch, the search for similarity method of Pearson and Lipman, and optionally by computerized implementations of these algorithms such as GAP, BESTFIT, FASTA, and TFASTA available as part of the GCG® Wisconsin Package® (Accelrys Inc., San Diego, Calif.). An “identity fraction” for aligned segments of a test sequence and a reference sequence is the number of identical components which are shared by the two aligned sequences divided by the total number of components in the reference sequence segment, i.e., the entire reference sequence or a smaller defined part of the reference sequence. Percent sequence identity is represented as the identity fraction multiplied by 100. The comparison of one or more polynucleotide sequences may be to a full-length polynucleotide sequence or a portion thereof, or to a longer polynucleotide sequence. For purposes of this invention “percent identity” may also be determined using BLASTX version 2.0 for translated nucleotide sequences and BLASTN version 2.0 for polynucleotide sequences.

Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., 1990). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when the cumulative alignment score falls off by the quantity X from its maximum achieved value, the cumulative score goes to zero or below due to the accumulation of one or more negative-scoring residue alignments, or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, a cutoff of 100, M=5, N=−4, and a comparison of both strands. For amino acid sequences, the BLASTP program uses as defaults a wordlength (W) of 3, an expectation (E) of 10, and the BLOSUM62 scoring matrix (see Henikoff & Henikoff, Proc. Natl. Acad. Sci. USA 89: 10915 (1989)).

In addition to calculating percent sequence identity, the BLAST algorithm also performs a statistical analysis of the similarity or identity between two sequences (see, e.g., Karlin & Altschul, Proc. Nat'l. Acad. Sci. USA 90: 5873-5787 (1993)). One measure of similarity or identity provided by the BLAST algorithm is the smallest sum probability (P(N)), which provides an indication of the probability by which a match between two nucleotide or amino acid sequences would occur by chance. For example, a test nucleic acid sequence is considered similar to a reference sequence if the smallest sum probability in a comparison of the test nucleotide sequence to the reference nucleotide sequence is less than about 0.1 to less than about 0.001. Thus, in some embodiments of the invention, the smallest sum probability in a comparison of the test nucleotide sequence to the reference nucleotide sequence is less than about 0.001.

Two nucleotide sequences may also be considered to be substantially complementary when the two sequences hybridize to each other under stringent conditions. In some embodiments, two nucleotide sequences considered to be substantially complementary hybridize to each other under highly stringent conditions.

As used herein, the terms “transfection” and “transduction” refer to the uptake of an exogenous nucleic acid (RNA and/or DNA) by a plant cell. A cell has been “transfected” or “transduced” with an exogenous nucleic acid when such nucleic acid has been introduced or delivered into the cell. A cell has been “transformed” by an exogenous nucleic acid when the transfected or transduced nucleic acid imparts a phenotypic change to the cell and/or a change in an activity or function of the cell. The transforming nucleic acid can be integrated (covalently linked) into chromosomal DNA making up the genome of the cell or it can be present as a stable plasmid.

As used herein, the terms “transgenic” and “recombinant” refer to an organism (e.g., a bacterium or plant) that comprises one or more exogenous nucleic acids. Generally, the exogenous nucleic acid is stably integrated within the genome such that at least a portion of the exogenous nucleic acid is passed on to successive generations. The exogenous nucleic acid may be integrated into the genome alone or as part of a recombinant expression cassette. “Transgenic” may be used to designate any organism the genotype of which has been altered by the presence of an exogenous nucleic acid, including those transgenics initially so altered and those created by sexual crosses or asexual propagation from the initial transgenic. As used herein, the term “transgenic” does not encompass the alteration of the genome (chromosomal or extra-chromosomal) by conventional breeding methods or by naturally occurring events such as random cross-fertilization, non-recombinant viral infection, non-recombinant bacterial transformation, non-recombinant transposition or spontaneous mutation.

As used herein, the term “vector” refers to a nucleic acid molecule for the cloning of and/or transfer of a nucleic acid into a cell. A vector may be a replicon to which another nucleotide sequence may be attached to allow for replication of the attached nucleotide sequence. A “replicon” can be any genetic element (e.g., plasmid, phage, cosmid, chromosome, viral genome) that functions as an autonomous unit of nucleic acid replication in vivo (i.e., is capable of replication under its own control). The term “vector” includes both viral and nonviral (e.g., plasmid) nucleic acid molecules for introducing a nucleic acid into a cell in vitro, ex vivo, and/or in vivo. A large number of vectors known in the art may be used to manipulate nucleic acids, incorporate response elements and promoters into genes, etc. For example, the insertion of nucleic acid fragments corresponding to response elements and promoters into a suitable vector can be accomplished by ligating the appropriate nucleic acid fragments into a chosen vector that has complementary cohesive termini Alternatively, the ends of the nucleic acid molecules may be enzymatically modified or any site may be produced by ligating nucleotide sequences (linkers) to the nucleic acid termini Such vectors may be engineered to contain sequences encoding selectable markers that provide for the selection of cells that contain the vector and/or have incorporated the nucleic acid of the vector into the cellular genome. Such markers allow identification and/or selection of host cells that incorporate and express the proteins encoded by the marker. Examples of such markers are disclosed in Messing & Vierra., GENE 19: 259-268 (1982); Bevan et al., NATURE 304:184-187 (1983); White et al., NUCL. ACIDS RES. 18: 1062 (1990); Spencer et al., THEOR. APPL. GENET. 79: 625-631 (1990); Blochinger & Diggelmann, MOL. CELL BIOL. 4: 2929-2931 (1984); Bourouis et al., EMBO J. 2(7): 1099-1104 (1983); U.S. Pat. No. 4,940,935; U.S. Pat. No. 5,188,642; U.S. Pat. No. 5,767,378; and U.S. Pat. No. 5,994,629. A “recombinant” vector refers to a viral or non-viral vector that comprises one or more heterologous nucleotide sequences (i.e., transgenes). Vectors may be introduced into cells by any suitable method known in the art, including, but not limited to, transfection, electroporation, microinjection, transduction, cell fusion, DEAE dextran, calcium phosphate precipitation, lipofection (lysosome fusion), and use of a gene gun or nucleic acid vector transporter.

As used herein, the term “yield reduction” (YD) refers to the degree to which yield is reduced in plants grown under stress conditions. YD is calculated as:

$$\frac{\begin{matrix}yield under non \text{-} stress conditions - \\ yield under stress conditons\end{matrix}}{yield under non \text{-} stress conditions} \times 100$$

The present invention provides compositions and methods useful for controlling a variety of pests, including, but not limited to, pests from:

the order Acarina (e.g., Acarus siro, Aceria sheldoni, Aculus schlechtendali, Amblyomma spp., Argas spp., Boophilus spp., Brevipalpus spp., Bryobia praetiosa, Calipitrimerus spp., Chorioptes spp., Dermanyssus gallinae, Eotetranychus carpini, Eriophyes spp., Hyalomma spp., Ixodes spp., Olygonychus pratensis, Ornithodoros spp., Panonychus spp., Phyllocoptruta oleivora, Polyphagotarsonemus latus, Psoroptes spp., Rhipicephalus spp., Rhizoglyphus spp., Sarcoptes spp., Tarsonemus spp. and Tetranychus spp.);

the order Anoplura (e.g., Haematopinus spp., Linognathus spp., Pediculus spp., Pemphigus spp. and Phylloxera spp.);

the order Coleoptera (e.g., Agriotes spp., Anthonomus spp., Atomaria linearis, Chaetocnema tibialis, Cosmopolites spp., Curculio spp., Dermestes spp., Diabrotica spp., Epilachna spp., Eremnus spp., Leptinotarsa decemLineata, Lissorhoptrus spp., Melolontha spp., Orycaephilus spp., Otiorhynchus spp., Phlyctinus spp., Popillia spp., Psylliodes spp., Rhizopertha spp., Scarabeidae, Sitophilus spp., Sitotroga spp., Tenebrio spp., Tribolium spp. and Trogoderma spp.);

the order Dermaptera (e.g., Forficula spp. and Labidura spp.);

the order Diptera (e.g., Aedes spp., Antherigona soccata, Bibio hortulanus, Calliphora erythrocephala, Ceratitis spp., Chrysomyia spp., Culex spp., Cuterebra spp., Dacus spp., Drosophila melanogaster, Fannia spp., Gastrophilus spp., Glossina spp., Hypoderma spp., Hyppobosca spp., Liriomyza spp., Lucilia spp., Melanagromyza spp., Musca spp., Oestrus spp., Orseolia spp., Oscinella frit, Pegomyia hyoscyami, Phorbia spp., Rhagoletis pomonella, Sciara spp., Stomoxys spp., Tabanus spp., Tannia spp. and Tipula spp.);

the order Hemiptera (e.g., Blissus spp., Lygus spp., Acrosternum spp. and Euschistus spp.);

the order Heteroptera (e.g., Cimex spp., Distantiella theobroma, Dysdercus spp., Euchistus spp., Eurygaster spp., Leptocorisa spp., Nezara spp., Piesma spp., Rhodnius spp., Sahlbergella singularis, Scotinophara spp. and Triatoma spp.);

the order Homoptera (e.g., Aleurothrixus floccosus, Aleyrodes brassicae, Aonidiella spp., Aphididae, Aphis spp., Aspidiotus spp., Bemisia tabaci, Ceroplaster spp., Chrysomphalus aonidium, Chrysomphalus dictyospermi, Coccus hesperidum, Empoasca spp., Eriosoma larigerum, Erythroneura spp., Gascardia spp., Laodelphax spp., Lecanium corni, Lepidosaphes spp., Macrosiphus spp., Myzus spp., Nephotettix spp., Nilaparvata spp., Parlatoria spp., Pemphigus spp., Planococcus spp., Pseudaulacaspis spp., Pseudococcus spp., Psylla spp., Pulvinaria aethiopica, Quadraspidiotus spp., Rhopalosiphum spp., Saissetia spp., Scaphoideus spp., Schizaphis spp., Sitobion spp., Trialeurodes vaporariorum, Trioza erytreae and Unaspis citri);

the order Hymenoptera (e.g., Acromyrmex, Atta spp., Cephus spp., Diprion spp., Diprionidae, Gilpinia polytoma, Hoplocampa spp., Lasius spp., Monomorium pharaonic, Neodiprion spp., Solenopsis spp. and Vespa spp.);

the order Isoptera (e.g., Reticulitermes spp.);

the order Lepidoptera (e.g., Acleris spp., Adoxophyes spp., Aegeria spp., Agrotis spp., Alabama argillaceae, Amylois spp., Anticarsia gemmatalis, Archips spp., Argyrotaenia spp., Autographa spp., Busseola fusca, Cadra cautella, Carposina nipponensis, Chilo spp., Choristoneura spp., Clysia ambiguella, Cnaphalocrocis spp., Cnephasia spp., Cochylis spp., Coleophora spp., Crocidolomia binotalis, Cryptophlebia leucotreta, Cydia spp., Diatraea spp., Diparopsis castanea, Earias spp., Ephestia spp., Eucosma spp., Eupoecilia ambiguella, Euproctis spp., Euxoa spp., Grapholita spp., Hedya nubiferana, Heliothis spp., Hellula undalis, Hyphantria cunea, Keiferia lycopersicella, Leucoptera scitella, Lithocollethis spp., Lobesia botrana, Lymantria spp., Lyonetia spp., Malacosoma spp., Mamestra brassicae, Manduca sexta, Operophtera spp., Ostrinia nubilalis, Pammene spp., Pandemis spp., Panolis flammea, Pectinophora gossypiela, Phthorimaea operculella, Pieris rapae, Pieris spp., Plutella xylostella, Prays spp., Scirpophaga spp., Sesamia spp., Sparganothis spp., Spodoptera spp., Synanthedon spp., Thaumetopoea spp., Tortrix spp., Trichoplusia ni and Yponomeuta spp.);

the order Mallophaga (e.g., Damalinea spp. and Trichodectes spp.);

the order Orthoptera (e.g., Blatta spp., Blattella spp., Gryllotalpa spp., Leucophaea maderae, Locusta spp., Periplaneta spp. and Schistocerca spp.);

the order Psocoptera (e.g., Liposcelis spp.);

the order Siphoptera (e.g., Ceratophyllus spp., Ctenocephalides spp. and Xenopsylla cheopis);

the order Thysanoptera (e.g., Frankliniella spp., Hercinothrips spp., Scirtothrips aurantii, Taeniothrips spp., Thrips palmi and Thrips tabaci);

the order Thysanura (e.g., Lepisma saccharina); and

the order Trichoptera (e.g., Limnephilus spp.).

In some preferred embodiments, compositions and methods of the present invention may be used to control one or more of the following pests: Lepidoptera Ostrinia nubilalis (European corn borer), Agrotis ipsilon (black cutworm), Helicoverpa zea (corn earworm), Spodoptera frugiperda (fall armyworm), Diatraea grandiosella (southwestern corn borer), Elasmopalpus lignosellus (lesser cornstalk borer), Diatraea saccharalis (sugarcane borer), Heliohtis virescens (cotton bollworm), Scirpophaga incertulas (yellow stemborer), Chilo polychrysa (darkheaded riceborer), Mythimna separata (oriental armyworm), Chilo partellus (sorghum borer), Feltia subterranea (granulate cutworm), Homoeosoma electellum (sunflower head moth), Spodoptera exigua (beet armyworm), Pectinophora gossypiella (pink bollworm), Scirpophaga innotata (white stemborer), Cnaphalocrocis medinalis (leaffolder), Chilo plejadellus (rice stalk borer), Nymphula depunctalis (caseworm), Spodoptera litura (cutworm), Spodoptera mauritia (rice swarming caterpillar), Cochylis hospes (banded sunflower moth), Pseudaletia unipunctata (army worm), Agrotis orthogonia (pale western cutworm), Pseudoplusia includens (soybean looper), Anticarsia gemmatalis (velvetbean caterpillar), Plathypena scabra (green cloverworm), Coleoptera Diabrotica virgifera (western corn rootworm), Diabrotica longicornis (northern corn rootworm), Diabrotica undecimpunctata (southern corn rootworm), Cyclocephala borealis (northern masked chafer (white grub)), Cyclocephala immaculata (southern masked chafer (white grub)), Popillia japonica (Japanese beetle), Chaetocnema pulicaria (corn flea beetle), Sphenophorus maidis (maize billbug), Phyllophaga crinita (white grub), Melanotus spp. (wireworms), Eleodes spp. (wireworms), Conoderus spp. (wireworms), Aeolus spp. (wireworms), Oulema melanopus (cereal leaf beetle), Chaetocnema pulicaria (corn flea beetle), Oulema melanopus (cereal leaf beetle), Hypera punctata (clover leaf weevil), Anthonomus grandis (boll weevil), Colaspis brunnea (grape colaspis), Lissorhoptrus oryzophilus (rice water weevil), Sitophilus oryzae (rice weevil), Epilachna varivestis (Mexican bean beetle), Rhopalosiphum maidis (corn leaf aphid), Anuraphis maidiradicis (corn root aphid), Sipha flava (yellow sugarcane aphid), Schizaphis graminum (greenbug), Macrosiphum avenae (English grain aphid), Aphis gossypii (cotton aphid), Pseudatomoscelis seriatus (cotton fleahopper), Trialeurodes abutilonea (bandedwinged whitefly), Nephotettix nigropictus (rice leafhopper), Myzus persicae (green peach aphid), Empoasca fabae (potato leafhopper), Blissus leucopterus (chinch bug), Lygus lineolaris (tarnished plant bug), Acrosternum hilare (green stink bug), Euschistus servus (brown stink bug), Melanoplus femurrubrum (redlegged grasshopper), Melanoplus sanguinipes (migratory grasshopper), Melanoplus differentialis (differential grasshopper), Hylemya platura (seedcorn maggot), Agromyza parvicornis (corn blotch leafminer), Contarinia sorghicola (sorghum midge), Mayetiola destructor (Hessian fly), Sitodiplosis mosellana (wheat midge), Meromyza americana (wheat stem maggot), Hylemya coarctata (wheat bulb fly), Neolasioptera murtfeldtiana (sunflower seed midge), Anaphothrips obscurus (grass thrips), Frankliniella fusca (tobacco thrips), Thrips tabaci (onion thrips), and Sericothrips variabilis (soybean thrips).

The present invention encompasses nonnaturally occurring nucleic acids useful for enhancing pest resistance (e.g., Acarina and/or insect resistance) in a plant or plant part.

Nucleic acids of the present invention may comprise, consist essentially of or consist of a nucleotide sequence that encodes one or more δ-endotoxins and/or one or more chaperones for increasing the expression, stability and/or activity of one or more δ-endotoxins. In some embodiments, the nucleic acid encodes a polypeptide that comprises, consists essentially of or consists of an amino acid sequence that is at least about 70%, 75%, 80%, 85%, 90%, 91%, 92%, 93%, 94%, 95%, 96%, 97%, 98%, 99% 99.5% or more identical to the amino acid sequence of SEQ ID NO: 7 or to a functional fragment thereof In some embodiments, the nucleic acid encodes a polypeptide that comprises, consists essentially of or consists of an amino acid sequence that is at least about 70%, 75%, 80%, 85%, 90%, 91%, 92%, 93%, 94%, 95%, 96%, 97%, 98%, 99% 99.5% or more identical to the amino acid sequence of SEQ ID NO: 9 or to a functional fragment thereof. In some embodiments, the nucleic acid encodes a polypeptide that comprises, consists essentially of or consists of an amino acid sequence that is at least about 70%, 75%, 80%, 85%, 90%, 91%, 92%, 93%, 94%, 95%, 96%, 97%, 98%, 99% 99.5% or more identical to the amino acid sequence of SEQ ID NO: 8 and/or SEQ ID NO: 10 and/or to a functional fragment thereof.

In some embodiments, the nucleic acid comprises, consists essentially of or consists of:
(a) one or more of the nucleotide sequences set forth in SEQ ID NOs: 1-6;
(b) a nucleotide sequence that is at least about 70%, 75%, 80%, 85%, 90%, 91%, 92%, 93%, 94%, 95%, 96%, 97%, 98%, 99%, 99.5% or more identical to the nucleotide sequence set forth in SEQ ID NO: 1;
(c) a nucleotide sequence that is at least about 70%, 75%, 80%, 85%, 90%, 91%, 92%, 93%, 94%, 95%, 96%, 97%, 98%, 99%, 99.5% or more identical to the nucleotide sequence set forth in SEQ ID NO: 2;
(d) a nucleotide sequence that is at least about 70%, 75%, 80%, 85%, 90%, 91%, 92%, 93%, 94%, 95%, 96%, 97%, 98%, 99%, 99.5% or more identical to the nucleotide sequence set forth in SEQ ID NO: 3;
(e) a nucleotide sequence that is at least about 70%, 75%, 80%, 85%, 90%, 91%, 92%, 93%, 94%, 95%, 96%, 97%, 98%, 99%, 99.5% or more identical to the nucleotide sequence set forth in SEQ ID NO: 4;
(f) a nucleotide sequence that is at least about 70%, 75%, 80%, 85%, 90%, 91%, 92%, 93%, 94%, 95%, 96%, 97%, 98%, 99%, 99.5% or more identical to the nucleotide sequence set forth in SEQ ID NO: 5;
(g) a nucleotide sequence that is at least about 70%, 75%, 80%, 85%, 90%, 91%, 92%, 93%, 94%, 95%, 96%, 97%, 98%, 99%, 99.5% or more identical to the nucleotide sequence set forth in SEQ ID NO: 6;
(h) one or more nucleotide sequences that encode(s) a polypeptide comprising, consisting essentially of or consisting of the amino acid sequence set forth in any one of SEQ ID NOs: 7-10;
(i) a nucleotide sequence that encodes a polypeptide comprising, consisting essentially of or consisting of an amino acid sequence that is at least about 70%, 75%, 80%, 85%, 90%, 91%, 92%, 93%, 94%, 95%, 96%, 97%, 98%, 99%, 99.5% or more identical to the amino acid sequence set forth in SEQ ID NO: 7;
(j) a nucleotide sequence that encodes a polypeptide comprising, consisting essentially of or consisting of an amino acid sequence that is at least about 70%, 75%, 80%, 85%, 90%, 91%, 92%, 93%, 94%, 95%, 96%, 97%, 98%, 99%, 99.5% or more identical to the amino acid sequence set forth in SEQ ID NO: 8;
(k) a nucleotide sequence that encodes a polypeptide comprising, consisting essentially of or consisting of an amino acid sequence that is at least about 70%, 75%, 80%, 85%, 90%, 91%, 92%, 93%, 94%, 95%, 96%, 97%, 98%, 99%, 99.5% or more identical to the amino acid sequence set forth in SEQ ID NO: 9;
(l) a nucleotide sequence that encodes a polypeptide comprising, consisting essentially of or consisting of an amino acid sequence that is at least about 70%, 75%, 80%, 85%, 90%, 91%, 92%, 93%, 94%, 95%, 96%, 97%, 98%, 99%, 99.5% or more identical to the amino acid sequence set forth in SEQ ID NO: 10;
(m) a nucleotide sequence that is complementary to any one of the nucleotide sequences described in (a) to (1) above;
(n) a nucleotide sequence that hybridizes to any one of the nucleotide sequences described in (a) to (m) above under stringent hybridization conditions;
(o) a functional fragment of any one of the nucleotide sequences described in (a), (b), (d), (e), (g), (h), (i) and (k) above, wherein the functional fragment encodes a δ-endotoxin;
(p) a functional fragment of any one of the nucleotide sequences described in (a), (b), (d), (h) and (i) above, wherein the functional fragment encodes a polypeptide that comprises an N-terminal helical bundle domain that is at least about 70%, 75%, 80%, 85%, 90%, 91%, 92%, 93%, 94%, 95%, 96%, 97%, 98%, 99%, 99.5% or more identical to amino acids 72 to 286 of SEQ ID NO: 7, a central beta-sheet domain that is at least about 70%, 75%, 80%, 85%, 90%, 91%, 92%, 93%, 94%, 95%, 96%, 97%, 98%, 99%, 99.5% or more identical to amino acids 295 to 511 of SEQ ID NO: 7, and a C-terminal beta-sandwich domain that is at least about 70%, 75%, 80%, 85%, 90%, 91%, 92%, 93%, 94%, 95%, 96%, 97%, 98%, 99%, 99.5% or more identical to amino acids 514 to 675 of SEQ ID NO: 7;
(q) a functional fragment of any one of the nucleotide sequences described in (a), (e), (g), (h) and (k) above, wherein the functional fragment encodes a polypeptide that comprises an N-terminal helical bundle domain that is at least about 70%, 75%, 80%, 85%, 90%, 91%, 92%, 93%, 94%, 95%, 96%, 97%, 98%, 99%, 99.5% or more identical to amino acids 51 to 271 of SEQ ID NO: 9, a central beta-sheet domain that is at least about 70%, 75%, 80%, 85%, 90%, 91%, 92%, 93%, 94%, 95%, 96%, 97%, 98%, 99%, 99.5% or more identical to amino acids 279 to 481 of SEQ ID NO: 9, and a C-terminal beta-sandwich domain that is at least about 70%, 75%, 80%, 85%, 90%, 91%, 92%, 93%, 94%, 95%, 96%, 97%, 98%, 99%, 99.5% or more identical to amino acids 486 to 646 of SEQ ID NO: 9; and/or
(r) a functional fragment of any one of the nucleotide sequences described in (a), (c), (d), (f), (g), (h), (j) and (1) above, wherein the functional fragment encodes a protein the expression of which increases the expression, stability and/or activity of one or more δ-endotoxins.

In some preferred embodiments, the nucleic acid comprises a nucleotide sequence that encodes a protein having an amino acid sequence that is at least 70%, 75%, 80%, 85%, 90%, 91%, 92%, 93%, 94%, 95%, 96%, 97%, 98%, 99%, 99.5% or more identical to the amino acid sequence set forth in SEQ ID NO: 7 and a nucleotide sequence that encodes a protein having an amino acid sequence that is at least 70%, 75%, 80%, 85%, 90%, 91%, 92%, 93%, 94%, 95%, 96%, 97%, 98%, 99%, 99.5% or more identical to the amino acid sequence set forth in SEQ ID NO: 8. In some preferred embodiments, the nucleic acid comprises a nucleotide sequence that encodes a protein having an amino acid sequence that is at least 70%, 75%, 80%, 85%, 90%, 91%, 92%, 93%, 94%, 95%, 96%, 97%, 98%, 99%, 99.5% or more identical to the amino acid sequence set forth in SEQ ID NO: 9 and a nucleotide sequence that encodes a protein having an amino acid sequence that is at least 70%, 75%, 80%, 85%, 90%, 91%, 92%, 93%, 94%, 95%, 96%, 97%, 98%, 99%, 99.5% or more identical to the amino acid sequence set forth in SEQ ID NO: 10.

Nucleic acids of the present invention may comprise any suitable promoter sequence(s), including, but not limited to, constitutive promoters, tissue-specific promoters, chemically inducible promoters, wound-inducible promoters, stress-inducible promoters and developmental stage-specific promoters.

In some embodiments, the nucleic acid comprises one or more constitutive promoter sequences. For example, the nucleic acid may comprise one or more CaMV 19S, CaMV 35S, Arabidopsis At6669, maize H3 histone, rice actin 1, actin 2, rice cyclophilin, nos, Adh, sucrose synthase, pEMU, GOS2, constitutive root tip CT2, and/or ubiquitin (e.g., maize Ubi) promoter sequences. Examples of suitable promoters are disclosed in U.S. Pat. Nos. 5,352,605, 5,641, 876, 5,604,121, 6,040,504 and 7,166,770; WO 93/07278; WO 01/73087; EP 0342926; Binet et al., PLANT SCI. 79:87-94 (1991); Christensen et al., PLANT MOLEC. BIOL. 12: 619-632 (1989); Ebert et al., PROC. NATL. ACAD. SCI. USA 84:5745-5749 (1987); Norris et al., PLANT MOLEC. BIOL. 21:895-906 (1993); Walker et al., PROC. NATL. ACAD. SCI. USA 84:6624-6629 (1987); Wang et al., MOL. CELL. BIOL. 12:3399-3406 (1992); and Yang & Russell, PROC. NATL. ACAD. SCI. USA 87:4144-4148 (1990). Thus, in some embodiments, the nucleic acid comprises one or more of the nucleotide sequences described in (a) to (r) above operably linked to one or more constitutive promoters.

In some embodiments, the nucleic acid comprises one or more tissue-specific promoter sequences. For example, the nucleic acid may comprise one or more leaf-, ligule-, node-, internode-, panicle-, root-, seed-, sheath-, stem-, and/or vascular bundle-specific promoter sequences. Examples of suitable promoters are disclosed in U.S. Pat. Nos. 5,459,252, 5,604,121, 5,625,136, 6,040,504 and 7,579,516; EP 0452269; WO 93/07278; Czako et al., MOL. GEN. GENET. 235:33-40 (1992); Hudspeth & Grula, PLANT MOLEC. BIOL. 12:579-589 (1989); de Framond, FEBS 290:103-106 (1991); Jeong et al. PLANT PHYSIOL. 153:185-197 (2010); and Kim et al. PLANT CELL 18:2958-2970 (2006). Thus, in some embodiments, the nucleic acid comprises one or more of the nucleotide sequences described in (a) to (r) above operably linked to one or more tissue-specific promoters.

In some embodiments, the nucleic acid comprises one or more chemically inducible promoter sequences. Examples of suitable promoters are disclosed in U.S. Pat. Nos. 5,614,395, 5,789,156 and 5,814,618; EP 0332104; WO 97/06269; WO 97/06268; Aoyama et al., PLANT J. 11:605-612 (1997); De Cosa et al. NAT. BIOTECHNOL. 19:71-74 (2001); Daniell et al. BMC BIOTECHNOL. 9:33 (2009); Gatz et al. MOL. GEN. GENET. 227, 229-237 (1991); Gatz, CURRENT OPINION BIOTECHNOL. 7:168-172 (1996); Gatz, ANN. REV. PLANT PHYSIOL. PLANT MOL. BIOL. 48:89-108 (1997); Li et al., GENE 403:132-142 (2007); Li et al., MOL BIOL. REP. 37:1143-1154 (2010); McNellis et al. PLANT J. 14, 247-257 (1998); Muto et al. BMC BIOTECHNOL. 9:26 (2009); Schena et al. PROC. NATL. ACAD. SCI. USA 88, 10421-10425 (1991); Surzycki et al. BIOLOGICALS 37:133-138 (2009); and Walker et al. PLANT CELL REP. 23:727-735 (2005). Thus, in some embodiments, the nucleic acid comprises one or more of the nucleotide sequences described in (a) to (r) above operably linked to one or more chemically inducible promoters.

In some embodiments, the nucleic acid comprises one or more wound-inducible promoter sequences. Examples of suitable promoters are disclosed in Stanford et al., MOL. GEN. GENET. 215:200-208 (1989); Xu et al., PLANT MOLEC. BIOL. 22:573-588 (1993); Logemann et al., PLANT CELL 1:151-158 (1989); Rohrmeier & Lehle, PLANT MOLEC. BIOL. 22:783-792 (1993); Firek et al., PLANT MOLEC. BIOL. 22:129-142 (1993); and Warner et al., PLANT J. 3:191-201 (1993). Thus, in some embodiments, the nucleic acid comprises one or more of the nucleotide sequences described in (a) to (r) above operably linked to one or more wound-inducible promoters.

In some embodiments, the nucleic acid comprises one or more stress-inducible promoter sequences. For example, the nucleic acid may comprise one or more drought stress-inducible, salt stress-inducible, heat stress-inducible, light stress-inducible and/or osmotic stress-inducible promoter sequences. Thus, in some embodiments, the nucleic acid comprises one or more of the nucleotide sequences described in (a) to (r) above operably linked to one or more stress-inducible promoters.

In some embodiments, the nucleic acid comprises one or more developmental stage-specific promoter sequences. For example, the nucleic acid may comprise a promoter sequence that drives expression prior to and/or during the seedling, tillering, panicle initiation, panicle differentiation, reproductive, and/or grain filling stage(s) of development. Thus, in some embodiments, the nucleic acid comprises one or more of the nucleotide sequences described in (a) to (r) above operably linked to one or more developmental-stage specific promoters.

In some embodiments, the nucleic acid comprises one or more promoters useful for expression in bacteria and/or yeast. For example, the nucleic acid may comprise one or more yeast promoters associated with phosphoglycerate kinase (PGK), glyceraldehyde-3-phosphate dehydrogenase (GAP), triose phosphate isomerase (TPI), galactose-regulon (GAL1, GAL10), alcohol dehydrogenase (ADH1, ADH2), phosphatase (PHO5), copper-activated metallothionine (CUP1), MFa1, PGK/a2 operator, TPI/a2 operator, GAP/GAL, PGK/GAL, GAP/ADH2, GAP/PHOS, iso-1-cytochrome c/glucocorticoid response element (CYC/GRE), phosphoglycerate kinase/angrogen response element (PGK/ARE), transcription elongation factor EF-1α (TEF1), triose phosphate dehydrogenase (TDH3), phosphoglycerate kinase 1 (PGK1), pyruvate kinase 1 (PYK1), and/or hexose transporter (HXT7). Likewise, the nucleic acid may comprise any bacterial L-arabinose inducible (araBAD, P_{BAD}) promoter, lac promoter, L-rhamnose inducible (rhaP_{BAD}) promoter, T7 RNA polymerase promoter, trc promoter, tac promoter, lambda phage promoter (p_{L}, p_{L}-9G-50), anydrotetracycline-inducible (tetA) promoter, trp, lpp, phoA, recA, proU, cst-1, cadA, nar, cspA, T7-lac operator, T3-lac operator, T4 gene 32, T5-lac operator, nprM-lac operator, Vhb, Protein A, corynebacterial-E. coli like promoters, thr, horn, diphtheria toxin promoter, sig A, sig B, nusG, SoxS, katb, a-amylase (Parry), Ptms, P43 (comprised of two overlapping RNA polymerase a factor recognition sites, GA, GB), Ptms, P43, rplK-rplA, ferredoxin promoter, and/or xylose promoter. Examples of suitable promoters are disclosed in Hannig et al. TRENDS BIOTECHNOL. 16:54-60 (1998); Partow et al. YEAST 27:955-964 (2010); Romanos et al. YEAST 8:423-488 (1992); Srivastava et al., PROTEIN EXPR. PURIF. 40:221-229 (2005); Terpe, APPL. MICROBIOL, BIOTECHNOL. 72:211-222 (2006). Thus, in some embodiments, the nucleic acid comprises one or more of the nucleotide sequences described in (a) to (r) above operably linked to one or more yeast and/or bacterial promoters.

Nucleic acids of the present invention may comprise any suitable termination sequence(s). For example, the nucleic acid may comprise a termination sequence comprising a stop signal for RNA polymerase and a polyadenylation signal for polyadenylase. Thus, the nucleic acid comprises one or more of the nucleotide sequences described in (a) to (r) above operably linked to one or more termination sequences.

Nucleic acids of the present invention may comprise any suitable expression-enhancing sequence(s). For example, the nucleic acid may comprise one or more intron sequences (e.g., Adhl and/or bronzel) and/or viral leader sequences (from tobacco mosaic virus (TMV), tobacco etch virus (TEV), maize chlorotic mottle virus (MCMV), maize dwarf mottle virus (MDMV) or alfalfa mosaic virus (AMV), for example) that enhance expression of associated nucleotide sequences. Examples of suitable sequences are disclosed in Allison et al. VIROLOGY 154:9-20 (1986); Della-Cioppa et al. PLANT PHYSIOL. 84:965-968 (1987); Elroy-Stein et al. PROC. NATL. ACAD. SCI. USA 86:6126-6130 (1989); Gallie et al., GENE 165:233-238 (1995); Gallie et al. NUCLEIC ACIDS RES.15:8693-8711 (1987); Gallie et al. NUCLEIC ACIDS RES. 15:3257-3273 (1987); Gallie et al. NUCLEIC ACIDS RES. 16:883-893 (1988); Gallie et al. NUCLEIC ACIDS RES. 20:4631-4638 (1992); Jobling et al. NATURE 325:622-625 (1987); Lommel et al. VIROLOGY 81:382-385 (1991); Skuzeski et al., PLANT MOLEC. BIOL. 15:65-79 (1990). Thus, the nucleic acid comprises one or more of the nucleotide sequences described in (a) to (r) above operably linked to one or more expression-enhancing sequences.

Nucleic acids of the present invention may comprise any suitable transgene(s), including, but not limited to, transgenes that encode gene products that provide enhanced abiotic stress tolerance (e.g., enhanced drought stress tolerance, enhanced osmotic stress tolerance, enhanced salt stress tolerance and/or enhanced temperature stress tolerance), herbicide-resistance (e.g., enhanced glyphosate-, Sulfonylurea-, imidazolinione-, dicamba-, glufisinate-, phenoxy proprionic acid-, cycloshexome-, traizine-, benzonitrile-, and/or broxynil-resistance), pest-resistance and/or disease-resistance.

Nucleic acids of the present invention may encode any suitable epitope tag, including, but not limited to, poly-Arg tags (e.g., RRRRR and RRRRRR) and poly-His tags (e.g., HHHHHH). In some embodiments, the nucleic acid comprises a nucleotide sequence encoding a poly-Arg tag, a poly-His tag, a FLAG tag (i.e., DYKDDDDK), a Strep-tag IITM (GE Healthcare, Pittsburgh, PA, USA) (i.e., WSHPQFEK), and/or a c-myc tag (i.e., EQKLISEEDL).

Nucleic acids of the present invention may comprise any suitable number of nucleotides. In some embodiments, the nucleic acid is 1500, 1550, 1600, 1650, 1700, 1750, 1800, 1850, 1900, 1950, 2000, 2050, 2100, 2150, 2200, 2250, 2300, 2350, 2400, 2450, 2500, 2550, 2600, 2650, 2700, 2750, 2800, 2850, 2900, 2950, 3000, 3050, 3100, 3150, 3200, 3250, 3300, 3350, 3400, 3450, 3500, 3550, 3600, 3650, 3700, 3750, 3800, 3850, 3900, 3950, 4000 or more nucleotides in length. In some embodiments, the nucleic acid is less than about 1500, 1550, 1600, 1650, 1700, 1750, 1800, 1850, 1900, 1950, 2000, 2050, 2100, 2150, 2200, 2250, 2300, 2350, 2400, 2450, 2500, 2550, 2600, 2650, 2700, 2750, 2800, 2850, 2900, 2950, 3000, 3050, 3100, 3150, 3200, 3250, 3300, 3350, 3400, 3450, 3500, 3550, 3600, 3650, 3700, 3750, 3800, 3850, 3900, 3950, 4000 nucleotides in length. In some embodiments, the nucleic acid is about 1500, 1550, 1600, 1650, 1700, 1750, 1800, 1850, 1900, 1950, 2000, 2050, 2100, 2150, 2200, 2250, 2300, 2350, 2400, 2450, 2500, 2550, 2600, 2650, 2700, 2750, 2800, 2850, 2900, 2950, 3000, 3050, 3100, 3150, 3200, 3250, 3300, 3350, 3400, 3450, 3500, 3550, 3600, 3650, 3700, 3750, 3800, 3850, 3900, 3950, 4000 nucleotides in length.

Nucleic acids of the present invention may be codon optimized for expression in bacteria, viruses, fungi or plants. Codon optimization is well known in the art and involves modification of a nucleotide sequence for codon usage bias using species-specific codon usage tables. The codon usage tables are generated based on a sequence analysis of the most highly expressed genes for the species of interest. When the nucleotide sequences are to be expressed in the nucleus, the codon usage tables are generated based on a sequence analysis of highly expressed nuclear genes for the species of interest. The modifications of the nucleotide sequences are determined by comparing the species specific codon usage table with the codons present in the native polynucleotide sequences. As is understood in the art, codon optimization of a nucleotide sequence results in a nucleotide sequence having less than 100% identity (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, and the like) to the native nucleotide sequence but which still encodes a polypeptide having the same function as that encoded by the original, native nucleotide sequence. Thus, in some embodiments of the present invention, the nucleic acid molecule may be codon optimized for expression in a particular species of interest (e.g., a plant such as maize, soybean, sugar cane, sugar beet, rice or wheat).

Because expression levels may also be dependent on GC content, nucleic acids of the present invention may also be GC-optimized That is, the nucleotide sequences of nucleic acids of the present invention may be selectively altered to optimize their GC content for increased expression in the desired organism. For example, because microbial nucleotide sequences that have low GC contents may express poorly in plants due to the existence of ATTTA motifs that may destabilize messages and/or AATAAA motifs that may cause inappropriate polyadenylation, expression in plants may be enhanced by increasing GC content to at least about 35%, 40%, 45%, 50%, 55%, 60%, 65%, 70%, 75% or more.

In some embodiments, nucleic acids of the present invention are isolated nucleic acids.

The present invention also encompasses expression cassettes comprising one or more nucleic acids of the present invention. In some embodiments, the expression cassette comprises a nucleic acid that confers at least one property (e.g., resistance to a selection agent) that can be used to detect, identify or select transformed plant cells and tissues.

The present invention also encompasses vectors comprising one or more nucleic acids and/or expression cassettes of the present invention. In some embodiments, the vector is a pSTK, pROKI, pBin438, pCAMBIA (e.g., pCAMBIA1302, pCAMBIA2301, pCAMBIA1301, pCAMBIA1391-Xa, pCAMBIA1391-Xb) (CAMBIA Co., Brisbane, Australia) or pBI121 vector.

The present invention also encompasses transgenic cells/organisms comprising one or more nucleic acids, expression cassettes and/or vectors of the present invention. In some embodiments, the transgenic organism is a bacteria, virus, fungus, plant or plant part. In some embodiments, the transgenic cell is a fungal spore or fungal gamete. In some embodiments, the transgenic cell is a propagating plant cell, such as an egg cell or sperm cell. In some embodiments, the transgenic cell is a non-propagating plant cell.

The present invention also encompasses nonnaturally occurring proteins useful for enhancing pest resistance (e.g., Acarina and/or insect resistance) in a plant or plant part.

Proteins of the present invention may comprise any amino acid sequence the expression of which enhances the pest resistance of a plant or plant part.

In some embodiments, the protein is a pesticidal protein capable of controlling one or more pests. For example, the protein may be a pesticidal CRY71 protein comprising, consisting essentially of or consisting of an amino acid sequence that is at least about 70%, 75%, 80%, 85%, 90%, 91%, 92%, 93%, 94%, 95%, 96%, 97%, 98%, 99% 99.5% or more identical to the amino acid sequence of SEQ ID NO: 7 or to a functional fragment thereof. Such proteins may comprise an N-terminal helical bundle domain that is at least about 70%, 75%, 80%, 85%, 90%, 91%, 92%, 93%, 94%, 95%, 96%, 97%, 98%, 99%, 99.5% or more identical to amino acids 72 to 286 of SEQ ID NO: 7, a central beta-sheet domain that is at least about 70%, 75%, 80%, 85%, 90%, 91%, 92%, 93%, 94%, 95%, 96%, 97%, 98%, 99%, 99.5% or more identical to amino acids 295 to 511 of SEQ ID NO: 7, and/or a C-terminal beta-sandwich domain that is at least about 70%, 75%, 80%, 85%, 90%, 91%, 92%, 93%, 94%, 95%, 96%, 97%, 98%, 99%, 99.5% or more identical to amino acids 514 to 675 of SEQ ID NO: 7. Alternatively, the protein may be a pesticidal CRY72 protein comprising, consisting essentially of or consisting of an amino acid sequence that is at least about 70%, 75%, 80%, 85%, 90%, 91%, 92%, 93%, 94%, 95%, 96%, 97%, 98%, 99% 99.5% or more identical to the amino acid sequence of SEQ ID NO: 9 or to a functional fragment thereof. Such proteins may comprise an N-terminal helical bundle domain that is at least about 70%, 75%, 80%, 85%, 90%, 91%, 92%, 93%, 94%, 95%, 96%, 97%, 98%, 99%, 99.5% or more identical to amino acids 51 to 271 of SEQ ID NO: 9, a central beta-sheet domain that is at least about 70%, 75%, 80%, 85%, 90%, 91%, 92%, 93%, 94%, 95%, 96%, 97%, 98%, 99%, 99.5% or more identical to amino acids 279 to 481 of SEQ ID NO: 9, and/or a C-terminal beta-sandwich domain that is at least about 70%, 75%, 80%, 85%, 90%, 91%, 92%, 93%, 94%, 95%, 96%, 97%, 98%, 99%, 99.5% or more identical to amino acids 486 to 646 of SEQ ID NO: 9.

In some embodiments, the protein is an auxiliary protein capable of increasing the expression, stability and/or activity of one or more δ-endotoxins. For example, the protein may be an ORF2 protein comprising, consisting essentially of or consisting of an amino acid sequence that is at least about 70%, 75%, 80%, 85%, 90%, 91%, 92%, 93%, 94%, 95%, 96%, 97%, 98%, 99% 99.5% or more identical to the amino acid sequence of SEQ ID NO: 8 and/or SEQ ID NO: 10 or to a functional fragment thereof Such proteins may increase the expression, stability and/or activity of one of more δ-endotoxins (e.g., one or more Cry71 and/or Cry72 proteins) by, for example, acting as a molecular chaperone for the δ-endotoxin(s).

In some embodiments, the protein is a fusion protein, comprising, consisting essentially of, or consisting of a δ-endotoxin (e.g., a CRY71 or CRY72 protein) and an auxiliary protein capable of increasing the expression, stability and/or activity of the δ-endotoxin. For example, the protein may be a fusion protein comprising, consisting essentially of or consisting of an amino acid sequence that is at least about 70%, 75%, 80%, 85%, 90%, 91%, 92%, 93%, 94%, 95%, 96%, 97%, 98%, 99% 99.5% or more identical to the amino acid sequence of SEQ ID NO: 7 or to a functional fragment thereof and an amino acid sequence that is at least about 70%, 75%, 80%, 85%, 90%, 91%, 92%, 93%, 94%, 95%, 96%, 97%, 98%, 99% 99.5% or more identical to the amino acid sequence of SEQ ID NO: 8 or to a functional fragment thereof.

In some embodiments, the protein is an isolated protein.

Proteins of the present invention may comprise any suitable epitope tag, including, but not limited to, poly-Arg tags (e.g., RRRRR and RRRRRR) and poly-His tags (e.g., HHHHHH). In some embodiments, the nucleic acid comprises a nucleotide sequence encoding a poly-Arg tag, a poly-His tag, a FLAG tag (i.e., DYKDDDDK), a Strep-tag IITM (GE Healthcare, Pittsburgh, Pa., USA) (i.e., WSHPQFEK), and/or a c-myc tag (i.e., EQKLISEEDL).

Proteins of the present invention may comprise any suitable number of amino acids. In some embodiments, the proteins is 10, 15, 20, 25, 30, 35, 40, 45, 50, 55, 60, 65, 70, 75, 80, 85, 90, 95, 100, 125, 150, 175, 200, 225, 250, 275, 300, 325, 350, 375, 400, 450, 500, 550, 600, 650, 700, 750, 800, 850, 900, 950, 1000, 1050, 1100, 1150, 1200, 1200, 1250, 1300, 1350, 1400, 1450, 1500 or more amino acids in length. In some embodiments, the protein is less than about 10, 15, 20, 25, 30, 35, 40, 45, 50, 55, 60, 65, 70, 75, 80, 85, 90, 95, 100, 125, 150, 175, 200, 225, 250, 275, 300, 325, 350, 375, 400, 450, 500, 550, 600, 650, 700, 750, 800, 850, 900, 950, 1000, 1050, 1100, 1150, 1200, 1200, 1250, 1300, 1350, 1400, 1450, or 1500 amino acids in length. In some embodiments, the protein is about 10, 15, 20, 25, 30, 35, 40, 45, 50, 55, 60, 65, 70, 75, 80, 85, 90, 95, 100, 125, 150, 175, 200, 225, 250, 275, 300, 325, 350, 375, 400, 450, 500, 550, 600, 650, 700, 750, 800, 850, 900, 950, 1000, 1050, 1100, 1150, 1200, 1200, 1250, 1300, 1350, 1400, 1450, or 1500 amino acids in length.

Proteins of the present invention may be produced using any suitable means, including, but not limited to, expression of nucleic acids of the present invention in a transgenic organism. In some embodiments, proteins of the present invention are produced using a transgenic bacterium/fungus expressing one or more nucleic acids of the present invention under the control of one or more heterologous regulatory elements (e.g., the nucleotide sequence of SEQ ID NO: 1 under the control of a constitutive promoter suitable for use in Bt).

Proteins of the present invention may possess any suitable pesticidal activity.

In some embodiments, the protein is useful for controlling pests belonging to the order Acarina, pests belonging to the order Anoplura, pests belonging to the order Coleoptera, pests belonging to the order Dermaptera, pests belonging to the order Diptera, pests belonging to the order Hemiptera, pests belonging to the order Heteroptera, pests belonging to the order Homoptera, pests belonging to the order Hymenoptera, pests belonging to the order Isoptera, pests belonging to the order Lepidoptera, pests belonging to the order Mallophaga, pests belonging to the order Orthoptera, pests belonging to the order Psocoptera, pests belonging to the order Siphoptera, pests belonging to the order Thysanoptera, pests belonging to the order Thysanura, and/or pests belonging to the order Trichoptera

In some preferred embodiments, the protein is useful for controlling Lepidoptera Ostrinia nubilalis (European corn borer), Agrotis ipsilon (black cutworm), Helicoverpa zea (corn earworm), Spodoptera frugiperda (fall armyworm), Diatraea grandiosella (southwestern corn borer), Elasmopalpus lignosellus (lesser cornstalk borer), Diatraea saccharalis (sugarcane borer), Heliohtis virescens (cotton bollworm), Scirpophaga incertulas (yellow stemborer), Chilo polychrysa (darkheaded riceborer), Mythimna separata (oriental armyworm), Chilo partellus (sorghum borer), Feltia subterranea (granulate cutworm), Homoeosoma electellum (sunflower head moth), Spodoptera exigua (beet armyworm), Pectinophora gossypiella (pink bollworm), Scirpophaga innotata (white stemborer), Cnaphalocrocis medinalis (leaffolder), Chilo plejadellus (rice stalk borer), Nymphula depunctalis (caseworm), Spodoptera litura (cutworm), Spodoptera mauritia (rice swarming caterpillar), Cochylis hospes (banded sunflower moth), Pseudaletia unipunctata (army worm), Agrotis orthogonia (pale western cutworm), Pseudoplusia includens (soybean looper), Anticarsia gemmatalis (velvetbean caterpillar), Plathypena scabra (green cloverworm), Coleoptera Diabrotica virgifera (western corn rootworm), Diabrotica longicornis (northern corn rootworm), Diabrotica undecimpunctata (southern corn rootworm), Cyclocephala borealis (northern masked chafer (white grub)), Cyclocephala immaculata (southern masked chafer (white grub)), Popillia japonica (Japanese beetle), Chaetocnema pulicaria (corn flea beetle), Sphenophorus maidis (maize billbug), Phyllophaga crinita (white grub), Melanotus spp. (wireworms), Eleodes spp. (wireworms), Conoderus spp. (wireworms), Aeolus spp. (wireworms), Oulema melanopus (cereal leaf beetle), Chaetocnema pulicaria (corn flea beetle), Oulema melanopus (cereal leaf beetle), Hypera punctata (clover leaf weevil), Anthonomus grandis (boll weevil), Colaspis brunnea (grape colaspis), Lissorhoptrus oryzophilus (rice water weevil), Sitophilus oryzae (rice weevil), Epilachna varivestis (Mexican bean beetle), Rhopalosiphum maidis (corn leaf aphid), Anuraphis maidiradicis (corn root aphid), Sipha flava (yellow sugarcane aphid), Schizaphis graminum (greenbug), Macrosiphum avenae (English grain aphid), Aphis gossypii (cotton aphid), Pseudatomoscelis seriatus (cotton fleahopper), Trialeurodes abutilonea (bandedwinged whitefly), Nephotettix nigropictus (rice leafhopper), Myzus persicae (green peach aphid), Empoasca fabae (potato leafhopper), Blissus leucopterus (chinch bug), Lygus lineolaris (tarnished plant bug), Acrosternum hilare (green stink bug), Euschistus servus (brown stink bug), Melanoplus femurrubrum (redlegged grasshopper), Melanoplus sanguinipes (migratory grasshopper), Melanoplus differentialis (differential grasshopper), Hylemya platura (seedcorn maggot), Agromyza parvicornis (corn blotch leafminer), Contarinia sorghicola (sorghum midge), Mayetiola destructor (Hessian fly), Sitodiplosis mosellana (wheat midge), Meromyza americana (wheat stem maggot), Hylemya coarctata (wheat bulb fly), Neolasioptera murtfeldtiana (sunflower seed midge), Anaphothrips obscurus (grass thrips), Frankliniella fusca (tobacco thrips), Thrips tabaci (onion thrips), and/or Sericothrips variabilis (soybean thrips).

Proteins of the present invention may be used in combination with other pesticidal agents, including, but not limited to, other fungicidal, nematocidal and insecticidal agents. For example, proteins of the present invention may be used in combination with one or more biological insecticidal agents, such as vegetative insectidical proteins (e.g., Vip1, Vip2, Vip3, etc.), protease inhibitors, lectins, alpha-amylase, peroxidase, cholesterol oxidase and other Bt Cry proteins; and/or one or more chemical insecticidal agents, such as dinotefuran, thiamethoxam, imidacloprid, acetamiprid, nitenpyram, nidinotefuran, chlorfenapyr, tebufenpyrad, tebufenozide, methoxyfenozide, halofenozide, triazamate, avermectin, spinosad, fiprinol, acephate, fenamiphos, diazinon, chlorpyrifos, chlorpyrifon-methyl, malathion, carbaryl, aldicarb, carbofuran, thiodicarb, and oxamyl. In some embodiments, proteins of the present invention are used in combination with other Bacillus thuringiensis Cry proteins.

Nucleic acids and proteins of the present invention may be expressed in any suitable cell/organism, including, but not limited to, plants, bacteria, viruses and fungi. In some embodiments, the nucleic acid/protein is expressed in a monocot plant or plant part (e.g., in rice, maize, wheat, barley, oats, rye, millet, sorghum, fonio, sugar cane, bamboo, durum, kamut, triticale, secale, einkorn, spelt, emmer, teff, milo, flax, banana, ginger, onion, lily, daffodil, iris, amaryllis, orchid, canna, bluebell, tulip, garlic, gramma grass, Tripsacum sp., or teosinte). In some embodiments, the nucleic acid/protein is expressed in a dicot plant or plant part (e.g., in buckwheat, cotton, potato, quinoa, soybean, sugar beet, sunflower, tobacco or tomato).

Once a nucleotide sequence has been introduced into a particular cell/organism, it may be propagated in that species using traditional methods. Furthermore, once the nucleotide sequence has been introduced into a particular plant variety, it may be moved into other varieties (including commercial varieties) of the same species.

In some embodiments, the pest resistance of a plant or plant part expressing a nucleic acid/protein of the present invention is increased by at least about 5%, 10%, 15%, 20%, 25%, 30%, 35%, 40%, 45%, 50%, 55%, 60%, 65%, 75%, 80%, 85%, 90%, 95%, 100%, 125%, 150%, 175%, 200%, 250%, 300% or more as compared to a control plant or plant part (e.g., a native plant of the same species) grown under the same (or substantially the same) environmental conditions. For example, the Acarina-, Anoplura-, Coleoptera-, Dermaptera-, Diptera-, Hemiptera-, Heteroptera-, Homoptera-, Hymenoptera-, Isoptera-, Lepidoptera-, Mallophaga-, Orthoptera-, Psocoptera-, Siphoptera-, Thysanoptera-, Thysanura-, and/or Trichoptera-resistance of a plant or plant part expressing a nucleic acid encoding one or more CRY71 and/or CRY72 proteins (e.g., a CRY71 protein have an amino acid sequence that is at least 90%, 91%, 92%, 93%, 94%, 95%, 96%, 97%, 98%, 99%, 99.5% or more identical to SEQ ID NO: 7 and/or a CRY72 protein have an amino acid sequence that is at least 90%, 91%, 92%, 93%, 94%, 95%, 96%, 97%, 98%, 99%, 99.5% or more identical to SEQ ID NO: 9) may be increased by at least about 5%, 10%, 15%, 20%, 25%, 30%, 35%, 40%, 45%, 50%, 55%, 60%, 65%, 75%, 80%, 85%, 90%, 95%, 100%, 125%, 150%, 175%, 200%, 250%, 300% or more as compared to a control plant or plant part (e.g., a native plant or plant part of the same species) grown under the same (or substantially the same) pest stress conditions. Co-expression of one or more ORF2 proteins may further enhance the pest resistance of such plants and plant parts. Thus, in some embodiments, the Acarina-, Anoplura-, Coleoptera-, Dermaptera-, Diptera-, Hemiptera-, Heteroptera-, Homoptera-, Hymenoptera-, Isoptera-, Lepidoptera-, Mallophaga-, Orthoptera-, Psocoptera-, Siphoptera-, Thysanoptera-, Thysanura-, and/or Trichoptera-resistance of a plant or plant part expressing one or more CRY71 and/or CRY72 proteins as well as one or more ORF2 proteins may be increased by at least about 10%, 15%, 20%, 25%, 30%, 35%, 40%, 45%, 50%, 55%, 60%, 65%, 75%, 80%, 85%, 90%, 95%, 100%, 125%, 150%, 175%, 200%, 250%, 300%, 350%, 400%, 450%, 500%, 550%, 600% or more as compared to a plant or plant part (e.g., a native plant of the same species) grown under the same (or substantially the same) pest stress conditions. For example, the Acarina-, Anoplura-, Coleoptera-, Dermaptera-, Diptera-, Hemiptera-, Heteroptera-, Homoptera-, Hymenoptera-, Isoptera-, Lepidoptera-, Mallophaga-, Orthoptera-, Psocoptera-, Siphoptera-, Thysanoptera-, Thysanura-, and/or Trichoptera-resistance of a plant or plant part expressing SEQ ID NO: 7 and SEQ ID NO: 8 (either separately from SEQ ID NO: 1 and SEQ ID NO: 2, or collectively from SEQ ID NO: 3) may be increased by at least about 10%, 15%, 20%, 25%, 30%, 35%, 40%, 45%, 50%, 55%, 60%, 65%, 75%, 80%, 85%, 90%, 95%, 100%, 125%, 150%, 175%, 200%, 250%, 300%, 350%, 400%, 450%, 500%, 550%, 600% or more as compared to a control plant or plant part grown under the same (or substantially the same) pest stress conditions. Similarly, the Acarina-, Anoplura-, Coleoptera-, Dermaptera-, Diptera-, Hemiptera-, Heteroptera-, Homoptera-, Hymenoptera-, Isoptera-, Lepidoptera-, Mallophaga-, Orthoptera-, Psocoptera-, Siphoptera-, Thysanoptera-, Thysanura-, and/or Trichoptera-resistance of a plant or plant part expressing SEQ ID NO: 9 and SEQ ID NO: 10 (either separately from SEQ ID NO: 4 and SEQ ID NO: 5, or collectively from SEQ ID NO: 6) may be increased by at least about 10%, 15%, 20%, 25%, 30%, 35%, 40%, 45%, 50%, 55%, 60%, 65%, 75%, 80%, 85%, 90%, 95%, 100%, 125%, 150%, 175%, 200%, 250%, 300%, 350%, 400%, 450%, 500%, 550%, 600% or more as compared to a control plant or plant part grown under the same (or substantially the same) pest stress conditions.

Plants and plant parts expressing nucleic acids/proteins of the present invention may exhibit a variety of pest resistant phenotypes, including, but not limited to, increased survival rate, increased growth rate, increased height and/or increased yield (e.g., increased biomass, increased seed yield, increased YGSMN, increased GMSTP, increased GWTPN, increased percent PYREC, decreased YRED, and/or decreased PB) when grown under pest stress conditions (e.g., Acarina and/or insect infestation). In some embodiments, one or more pest resistant phenotypes is increased by at least about 5%, 10%, 15%, 20%, 25%, 30%, 35%, 40%, 45%, 50%, 55%, 60%, 65%, 75%, 80%, 85%, 90%, 95%, 100%, 125%, 150%, 175%, 200%, 250%, 300%, or more as compared to a control plant or plant part (e.g., a native plant of the same species) when each is grown under the same (or substantially the same) environmental conditions.

In some embodiments, the yield (e.g., seed yield, biomass, GWTPN, PYREC and/or YGSMN) of a plant or plant part expressing a nucleic acid/protein of the present invention is increased by at least about 5%, 10%, 15%, 20%, 25%, 30%, 35%, 40%, 45%, 50%, 55%, 60%, 65%, 75%, 80%, 85%, 90%, 95%, 100%, 125%, 150%, 175%, 200%, 250%, 300% or more as compared to a control plant or plant part (e.g., a native plant of the same species) grown under the same (or substantially the same) environmental conditions. For example, the seed yield and/or biomass of a plant or plant part expressing SEQ ID NO: 1, SEQ ID NO: 3, SEQ ID NO: 4 and/or SEQ ID NO: 6 may be increased by at least about 5%, 10%, 15%, 20%, 25%, 30%, 35%, 40%, 45%, 50%, 55%, 60%, 65%, 75%, 80%, 85%, 90%, 95%, 100%, 125%, 150%, 175%, 200%, 250%, 300% or more as compared to a control plant or plant part grown under the same (or substantially the same) pest stress conditions.

In some embodiments, the expression, stability and/or activity of one or more δ-endotoxins in a plant or plant part expressing a nucleic acid/protein of the present invention is increased by at least about 5%, 10%, 15%, 20%, 25%, 30%, 35%, 40%, 45%, 50%, 55%, 60%, 65%, 75%, 80%, 85%, 90%, 95%, 100%, 125%, 150%, 175%, 200%, 250%, 300% or more as compared to a control plant (e.g., a native plant of the same species) grown under the same (or substantially the same) environmental conditions. For example, the expression, stability and/or activity of one or more CRY71 proteins and/or one or more CRY72 proteins may be increased by at least about 5%, 10%, 15%, 20%, 25%, 30%, 35%, 40%, 45%, 50%, 55%, 60%, 65%, 75%, 80%, 85%, 90%, 95%, 100%, 125%, 150%, 175%, 200%, 250%, 300% or more in a plant or plant part expressing a nucleic acid that encodes any one of SEQ ID NOs: 8 and 10.

In some embodiments, it may be preferable to target expression of nucleotide acids of the present invention to different cellular localizations in the plant. In some cases, localization in the cytosol may be desirable, whereas in other cases, localization in some subcellular organelle may be preferred. Subcellular localization of transgene-encoded enzymes is undertaken using techniques well known in the art. Typically, a nucleotide sequence encoding a target peptide from a known organelle-targeted gene product is manipulated and fused upstream of the nucleotide sequence. Many such target sequences are known for the chloroplast and their functioning in heterologous constructions has been shown. The expression of the nucleotide sequences of the present invention is also targeted to the endoplasmic reticulum or to the vacuoles of the host cells. Techniques to achieve this are well known in the art.

In some embodiments, it may be desirable to target proteins of the present invention to particular parts of a cell such as the chloroplast, the cell wall, the mitochondria, and the like. A nucleotide sequence encoding a signal peptide may be operably linked at the 5′- or 3′-terminus of a heterologous nucleotide sequence or nucleic acid molecule.

Various mechanisms for targeting gene products are known to exist in plants and the sequences controlling the functioning of these mechanisms have been characterized in some detail. For example, the targeting of gene products to the chloroplast is controlled by a signal sequence found at the amino terminal end of various proteins, which is cleaved during chloroplast import to yield the mature protein (see, e.g., Comai et al., J. BIOL. CHEM. 263:15104-15109 (1988). These signal sequences may be fused to heterologous gene products to effect the import of heterologous products into the chloroplast (see, e.g., van den Broeck et al., NATURE 313:358-363(1985)). DNA encoding for appropriate signal sequences may be isolated from the 5′ end of the cDNAs encoding the RUBISCO protein, the CAB protein, the EPSP synthase enzyme, the GS2 protein and many other proteins that are known to be chloroplast localized.

The above-described targeting sequences may be utilized not only in conjunction with their endogenous promoters, but also in conjunction with heterologous promoters. Use of promoters that are heterologous to the targeting sequence not only provides the ability to target the sequence but also can provide an expression pattern that is different from that of the promoter from which the targeting signal is originally derived.

Signal peptides (and the targeting nucleotide sequences encoding them) are well known in the art and can be found in public databases such as the “Signal Peptide Website: An Information Platform for Signal Sequences and Signal Peptides.” (www.signalpeptide.de); the “Signal Peptide Database” (proline.bic.nus.edu.sg/spdb/index.html) (Choo et al., BMC BIOINFORMATICS 6:249 (2005)(available on www.biomedcentral.com/1471-2105/6/249/abstract); ChloroP (www.cbs.dtu.dk/services/ChloroP/; predicts the presence of chloroplast transit peptides (cTP) in protein sequences and the location of potential cTP cleavage sites); LipoP (www.cbs.dtu.dk/services/LipoP/; predicts lipoproteins and signal peptides in Gram negative bacteria); MITOPROT (ihg2.helmholtz-muenchen.de/ihg/mitoprot.html; predicts mitochondrial targeting sequences); PlasMit (gecco.org.chemie.uni-frankfurt.de/plasmit/index.html; predicts mitochondrial transit peptides in Plasmodium falciparum); Predotar (urgi.versailles.inra.fr/predotar/predotar.html; predicts mitochondrial and plastid targeting sequences); PTS1 (mendel.imp.ac.at/mendeljsp/sat/pts1/PTS1predictor.jsp; predicts peroxisomal targeting signal 1 containing proteins); SignalP (www.cbs.dtu.dk/services/SignalP/; predicts the presence and location of signal peptide cleavage sites in amino acid sequences from different organisms: Gram-positive prokaryotes, Gram-negative prokaryotes, and eukaryotes).

Thus, for example, to localize to a plastid, a transit peptide from plastidic Ferredoxin: NADP+ oxidoreductase (FNR) of spinach, which is disclosed in Jansen et al., CURRENT GENETICS 13:517-522 (1988), may be employed. In particular, the sequence ranging from the nucleotides −171 to 165 of the cDNA sequence disclosed therein may be used, which comprises the 5′ non-translated region as well as the sequence encoding the transit peptide. Another example of a transit peptide is that of the waxy protein of maize including the first 34 amino acid residues of the mature waxy protein (Klosgen et al. MOL. GEN. GENET. 217:155-161 (1989)). It is also possible to use this transit peptide without the first 34 amino acids of the mature protein. Furthermore, the signal peptides of the ribulose bisposphate carboxylase small subunit (Wolter et al. PROC. NATL. ACAD. SCI. USA 85:846-850 (1988); Nawrath et al. PROC. NATL. ACAD. SCI. USA 91:12760-12764 (1994)), of NADP malate dehydrogenase (Galiardo et al. PLANTA 197:324-332 (1995)), of glutathione reductase (Creissen et al. PLANT J. 8:167-175(1995)) and/or of the R1 protein (Lorberth et al. NATURE BIOTECHNOLOGY 16:473-477 (1998)) may be used.

The present invention also encompasses pesticidal compositions useful for controlling pests and enhancing pest resistance in plants and plant parts. Pesticidal compositions of the present invention may also be useful for protecting materials such as wood, leather, textiles, plastics, adhesives, paints, papers, floor coverings and building materials from pest infestation.

Pesticidal compositions of the present invention may comprise any suitable active ingredient(s).

In some embodiments, the active ingredient(s) comprise(s), consist(s) essentially of or consist(s) of a transgenic organism that expresses a nucleic acid/protein of the present invention. For example, the active ingredient may comprise a transgenic bacterium, virus or fungus that expresses a nucleic acid comprising, consisting essentially of or consisting of the nucleotide sequence(s) of SEQ ID NO: 1, SEQ ID NO: 3, SEQ ID NO: 4 and/or SEQ ID NO: 6. In some embodiments, the active ingredient(s) comprise(s), consist(s) essentially of or consist(s) of one or more proteins of the present invention. For example, the active ingredient may comprise one or more isolated CRY71 proteins having an amino acid sequence that is at least 90%, 91%, 92%, 93%, 94%, 95%, 96%, 97%, 98%, 99%, 99.5% or more identical to SEQ ID NO: 7 and/or one or more isolated CRY72 proteins having an amino acid sequence that is at least 90%, 91%, 92%, 93%, 94%, 95%, 96%, 97%, 98%, 99%, 99.5% or more identical to SEQ ID NO: 9.

As noted above, proteins of the present invention may be used in combination with other pesticidal agents. Thus, in some embodiments, the pesticidal compositions comprises one or more of the aforementioned active ingredients in combination with one or more other pesticidal agents (e.g., one or more fungicidal agents, one or more nematocidal agents, one or more insecticidal agents and/or one more Acarinacidal agents). For example, proteins of the present invention may be used in combination with one or more biological insecticidal agents, such as vegetative insectidical proteins (e.g., Vip1, Vip2, Vip3, etc.), protease inhibitors, lectins, alpha-amylase, peroxidase, cholesterol oxidase and other Bt Cry proteins; and/or one or more chemical insecticidal agents, such as dinotefuran, thiamethoxam, imidacloprid, acetamiprid, nitenpyram, nidinotefuran, chlorfenapyr, tebufenpyrad, tebufenozide, methoxyfenozide, halofenozide, triazamate, avermectin, spinosad, fiprinol, acephate, fenamiphos, diazinon, chlorpyrifos, chlorpyrifon-methyl, malathion, carbaryl, aldicarb, carbofuran, thiodicarb, and oxamyl. In some embodiments, proteins of the present invention are used in combination with other Bacillus thuringiensis Cry proteins.

Pesticidal compositions of the present invention may comprise any suitable auxiliaries, including, but not limited to, one or more solvents, solid carriers, absorptive carriers, and/or surfactants.

The present invention therefore encompasses pesticidal compositions such as emulsifiable concentrates, suspension concentrates, directly sprayable or dilutable solutions, spreadable pastes, dilute emulsions, soluble powders, dispersible powders, wettable powders, dusts, granules and encapsulations in polymeric substances. Examples of suitable solvents are unhydrogenated or partially hydrogenated aromatic hydrocarbons, preferably the fractions C₈ ^{to} C₁₂ of alkylbenzenes, such as xylene mixtures, alkylated naphthalenes or tetrahydronaphthalene, aliphatic or cycloaliphatic hydrocarbons, such as paraffins or cyclohexane, alcohols such as ethanol, propanol or butanol, glycols and their ethers and esters such as propylene glycol, dipropylene glycol ether, ethylene glycol or ethylene glycol monomethyl ether or ethylene glycol monoethyl ether, ketones, such as cyclohexanone, isophorone or diacetone alcohol, strongly polar solvents, such as N-methylpyrrolid-2-one, dimethyl sulfoxide or N,N-dimethylformamide, water, unepoxidized or epoxidized vegetable oils, such as unexpodized or epoxidized rapeseed, castor, coconut or soya oil, and silicone oils.

Examples of suitable solid carriers are ground natural minerals, such as calcite, talc, kaolin, montmorillonite and attapulgite, highly dispersed silicas, highly dispersed absorbtive polymers, porous granuales, such as pumice, brick grit, sepiolite and bentonite, and non-sorptive carrier materials, such as calcite or sand. In addition, a large number of granulated materials of inorganic or organic nature can be used, in particular dolomite or comminuted plant residues.

Suitable surfactancts may be, depending on the type of the active ingredient to be formulated, non-ionic, cationic and/or anionic surfactants or surfactant mixtures which have good emulsifying, dispersing and wetting properties.

Examples of suitable non-ionic surfactants are polyglycol ether derivatives of aliphatic or cycloaliphatic alcohols, of saturated or unsaturated fatty acids or of alkyl phenols which may contain approximately 3 to approximately 30 glycol ether groups and approximately 8 to approximately 20 carbon atoms in the (cyclo)aliphatic hydrocarbon radical or approximately 6 to approximately 18 carbon atoms in the alkyl moiety of the alkyl phenols. Also suitable are water-soluble polyethylene oxide adducts with polypropylene glycol, ethylenediaminopolypropylene glycol or alkyl polypropylene glycol having 1 to approximately 10 carbon atoms in the alkyl chain and approximately 20 to approximately 250 ethylene glycol ether groups and approximately 10 to approximately 100 propylene glycol ether groups. Normally, the abovementioned compounds contain 1 to approximately 5 ethylene glycol units per propylene glycol unit. Examples which may be mentioned are nonylphenoxypolyethoxyethanol, castor oil polyglycol ether, polypropylene glycol/polyethylene oxide adducts, tributylphenoxypolyethoxyethanol, polyethylene glycol or octylphenoxypolyethoxyethanol. Also suitable are fatty acid esters of polyoxyethylene sorbitan, such as polyoxyethylene sorbitan trioleate.

Examples of suitable cationic surfactants are quarternary ammonium salts which generally have at least one alkyl radical of approximately 8 to approximately 22 C atoms as substituents and as further substituents (unhalogenated or halogenated) lower alkyl or hydroxyalkyl or benzyl radicals. Salts are preferably in the form of halides, methylsulfates or ethylsulfates. Examples are stearyltrimethylammonium chloride and benzylbis(2-chloroethyl)ethylammonium bromide.

Examples of suitable anionic surfactants are water-soluble soaps or water-soluble synthetic surface-active compounds. Examples of suitable soaps are the alkali, alkaline earth or (unsubstituted or substituted) ammonium salts of fatty acids having approximately 10 to approximately 22 C atoms, such as the sodium or potassium salts of oleic or stearic acid, or of natural fatty acid mixtures which are obtainable for example from coconut or tall oil; mention must also be made of the fatty acid methyl taurates. However, synthetic surfactants are used more frequently, in particular fatty sulfonates, fatty sulfates, sulfonated benzimidazole derivatives or alkylaryl sulfonates. As a rule, the fatty sulfonates and fatty sulfates are present as alkali, alkaline earth or (substituted or unsubstituted) ammonium salts and they generally have an alkyl radical of approximately 8 to approximately 22 C atoms, alkyl also to be understood as including the alkyl moiety of acyl radicals; examples which may be mentioned are the sodium or calcium salts of lignosulfonic acid, of the dodecylsulfuric ester or of a fatty alcohol sulfate mixture prepared from natural fatty acids. This group also includes the salts of the sulfuric esters and sulfonic acids of fatty alcohol/ethylene oxide adducts. The sulfonated benzimidazole derivatives preferably contain 2 sulfonyl groups and a fatty acid radical of approximately 8 to approximately 22 C atoms. Examples of alkylarylsulfonates are the sodium, calcium or triethanolammonium salts of decylbenzenesulfonic acid, of dibutylnaphthalenesulfonic acid or of a naphthalenesulfonic acid/formaldehyde condensate. Also possible are, furthermore, suitable phosphates, such as salts of the phosphoric ester of a pnonylphenol/(4-14)ethylene oxide adduct, or phospholipids.

Pesticidal compositions of the present invention may comprise about 0.1 to about 99% (e.g., about 0.1 to about 95%), by weight, active ingredient and about 1 to about 99.9% (e.g., about 5 to about 99.99%), by weight, of at least one solid or liquid adjuvant. In some embodiments, about 0 to about 25% (e.g., about 0.1 to about 20%), by weight, of the composition is surfactant. Whereas concentrated compositions may be preferred for commercial goods, the end consumer may dilute the pesticidal composition for use at a substantially lower concentration of active ingredient.

In some embodiments, pesticidal compositions of the present invention can comprise the following (%=percent by weight):

### Emulsifiable Concentrates

active ingredient: 1 to 95%, preferably 5 to 20%
surfactant: 1 to 30%, preferably 10 to 20%
solvent: 5 to 98%, preferably 70 to 85%

### Dusts

active ingredient: 0.1 to 10%, preferably 0.1 to 1%
solid carrier: 99.9 to 90%, preferably 99.9 to 99%

### Suspensions

active ingredient: 5 to 75%, preferably 10 to 50%
water: 94 to 24%, preferably 88 to 30%
surfactant: 1 to 40%, preferably 2 to 30%

### Wettable Powders

active ingredient: 0.5 to 90%, preferably 1 to 80%
surfactant: 0.5 to 20%, preferably 1 to 15%
solid carrier: 5 to 99%, preferably 15 to 98%

### Granulates

active ingredient: 0.5 to 30%, preferably 3 to 15%
solid carrier: 99.5 to 70%, preferably 97 to 85%

Pesticidal compositions of the present invention may be useful for controlling any suitable pest(s), including, but not limited to, pests belonging to the order Acarina, pests belonging to the order Anoplura, pests belonging to the order Coleoptera, pests belonging to the order Dermaptera, pests belonging to the order Diptera, pests belonging to the order Hemiptera, pests belonging to the order Heteroptera, pests belonging to the order Homoptera, pests belonging to the order Hymenoptera, pests belonging to the order Isoptera, pests belonging to the order Lepidoptera, pests belonging to the order Mallophaga, pests belonging to the order Orthoptera, pests belonging to the order Psocoptera, pests belonging to the order Siphoptera, pests belonging to the order Thysanoptera, pests belonging to the order Thysanura, and pests belonging to the order Trichoptera

In some preferred embodiments, the pesticidal composition is useful for controlling Lepidoptera Ostrinia nubilalis (European corn borer), Agrotis ipsilon (black cutworm), Helicoverpa zea (corn earworm), Spodoptera frugiperda (fall armyworm), Diatraea grandiosella (southwestern corn borer), Elasmopalpus lignosellus (lesser cornstalk borer), Diatraea saccharalis (sugarcane borer), Heliohtis virescens (cotton bollworm), Scirpophaga incertulas (yellow stemborer), Chilo polychrysa (darkheaded riceborer), Mythimna separata (oriental armyworm), Chilo partellus (sorghum borer), Feltia subterranea (granulate cutworm), Homoeosoma electellum (sunflower head moth), Spodoptera exigua (beet armyworm), Pectinophora gossypiella (pink bollworm), Scirpophaga innotata (white stemborer), Cnaphalocrocis medinalis (leaffolder), Chilo plejadellus (rice stalk borer), Nymphula depunctalis (caseworm), Spodoptera litura (cutworm), Spodoptera mauritia (rice swarming caterpillar), Cochylis hospes (banded sunflower moth), Pseudaletia unipunctata (army worm), Agrotis orthogonia (pale western cutworm), Pseudoplusia includens (soybean looper), Anticarsia gemmatalis (velvetbean caterpillar), Plathypena scabra (green cloverworm), Coleoptera Diabrotica virgifera (western corn rootworm), Diabrotica longicornis (northern corn rootworm), Diabrotica undecimpunctata (southern corn rootworm), Cyclocephala borealis (northern masked chafer (white grub)), Cyclocephala immaculata (southern masked chafer (white grub)), Popillia japonica (Japanese beetle), Chaetocnema pulicaria (corn flea beetle), Sphenophorus maidis (maize billbug), Phyllophaga crinita (white grub), Melanotus spp. (wireworms), Eleodes spp. (wireworms), Conoderus spp. (wireworms), Aeolus spp. (wireworms), Oulema melanopus (cereal leaf beetle), Chaetocnema pulicaria (corn flea beetle), Oulema melanopus (cereal leaf beetle), Hypera punctata (clover leaf weevil), Anthonomus grandis (boll weevil), Colaspis brunnea (grape colaspis), Lissorhoptrus oryzophilus (rice water weevil), Sitophilus oryzae (rice weevil), Epilachna varivestis (Mexican bean beetle), Rhopalosiphum maidis (corn leaf aphid), Anuraphis maidiradicis (corn root aphid), Sipha flava (yellow sugarcane aphid), Schizaphis graminum (greenbug), Macrosiphum avenae (English grain aphid), Aphis gossypii (cotton aphid), Pseudatomoscelis seriatus (cotton fleahopper), Trialeurodes abutilonea (bandedwinged whitefly), Nephotettix nigropictus (rice leafhopper), Myzus persicae (green peach aphid), Empoasca fabae (potato leafhopper), Blissus leucopterus (chinch bug), Lygus lineolaris (tarnished plant bug), Acrosternum hilare (green stink bug), Euschistus servus (brown stink bug), Melanoplus femurrubrum (redlegged grasshopper), Melanoplus sanguinipes (migratory grasshopper), Melanoplus differentialis (differential grasshopper), Hylemya platura (seedcorn maggot), Agromyza parvicornis (corn blotch leafminer), Contarinia sorghicola (sorghum midge), Mayetiola destructor (Hessian fly), Sitodiplosis mosellana (wheat midge), Meromyza americana (wheat stem maggot), Hylemya coarctata (wheat bulb fly), Neolasioptera murtfeldtiana (sunflower seed midge), Anaphothrips obscurus (grass thrips), Frankliniella fusca (tobacco thrips), Thrips tabaci (onion thrips), and/or Sericothrips variabilis (soybean thrips).

The present invention also encompasses amplification primers (and pairs of amplification primers) useful for isolating, amplifying and identifying CRY71 proteins, CRY72 proteins and ORF2 proteins.

Amplification primers of the present invention may comprise, consist essentially of or consists of:

(a) the nucleotide sequence set forth in SEQ ID NO: 11;

(b) the nucleotide sequence set forth in SEQ ID NO: 12;

(c) the nucleotide sequence set forth in SEQ ID NO: 13;

(d) the nucleotide sequence set forth in SEQ ID NO: 14;

(e) the nucleotide sequence set forth in SEQ ID NO: 15;

(f) the nucleotide sequence set forth in SEQ ID NO: 16;

(g) the nucleotide sequence set forth in SEQ ID NO: 17;

(h) the nucleotide sequence set forth in SEQ ID NO: 18;

(i) the nucleotide sequence set forth in SEQ ID NO: 19;

(j) the nucleotide sequence set forth in SEQ ID NO: 20;

(k) the nucleotide sequence set forth in SEQ ID NO: 21;

(l) the nucleotide sequence set forth in SEQ ID NO: 22; or

(m) a nucleotide sequence that is at least 90%, 91%, 92%, 93%, 94%, 95%, 96%, 97%, 98%, 99%, 99.5% or more identical to and hybridizes under stringent conditions to the complement of the nucleotide sequence of any one of SEQ ID NOs: 11 to 22.

Pairs of amplification primers useful for isolating, amplifying and identifying CRY71 proteins, CRY72 proteins and ORF2 proteins include, but are not limited to,

(a) the nucleotide sequence set forth in SEQ ID NO: 11 and the nucleotide sequence set forth in SEQ ID NO: 12;

(b) the nucleotide sequence set forth in SEQ ID NO: 13 and the nucleotide sequence set forth in SEQ ID NO: 14;

(c) the nucleotide sequence set forth in SEQ ID NO: 11 and the nucleotide sequence set forth in SEQ ID NO: 16;

(d) the nucleotide sequence set forth in SEQ ID NO: 15 and the nucleotide sequence set forth in SEQ ID NO: 16;

(e) the nucleotide sequence set forth in SEQ ID NO: 15 and the nucleotide sequence set forth in SEQ ID NO: 14;

(f) the nucleotide sequence set forth in SEQ ID NO: 17 and the nucleotide sequence set forth in SEQ ID NO: 18;

(g) the nucleotide sequence set forth in SEQ ID NO: 19 and the nucleotide sequence set forth in SEQ ID NO: 20;

(h) the nucleotide sequence set forth in SEQ ID NO: 17 and the nucleotide sequence set forth in SEQ ID NO: 22;

(i) the nucleotide sequence set forth in SEQ ID NO: 21 and the nucleotide sequence set forth in SEQ ID NO: 22;

(j) the nucleotide sequence set forth in SEQ ID NO: 21 and the nucleotide sequence set forth in SEQ ID NO: 20;

(k) a nucleotide sequence that is at least 90%, 91%, 92%, 93%, 94%, 95%, 96%, 97%, 98%, 99%, 99.5% or more identical to the nucleotide sequence set forth in SEQ ID NO: 11 and that hybridizes under stringent conditions to the complement of the nucleotide sequence set forth in SEQ ID NO: 11, and a nucleotide sequence that is at least 90%, 91%, 92%, 93%, 94%, 95%, 96%, 97%, 98%, 99%, 99.5% or more identical to the nucleotide sequence set forth in SEQ ID NO: 12 and that hybridizes under stringent conditions to the complement of the nucleotide sequence set forth in SEQ ID NO: 12;

(l) a nucleotide sequence that is at least 90%, 91%, 92%, 93%, 94%, 95%, 96%, 97%, 98%, 99%, 99.5% or more identical to the nucleotide sequence set forth in SEQ ID NO: 13 and that hybridizes under stringent conditions to the complement of the nucleotide sequence set forth in SEQ ID NO: 13, and a nucleotide sequence that is at least 90%, 91%, 92%, 93%, 94%, 95%, 96%, 97%, 98%, 99%, 99.5% or more identical to the nucleotide sequence set forth in SEQ ID NO: 14 and that hybridizes under stringent conditions to the complement of the nucleotide sequence set forth in SEQ ID NO: 14;

(m) a nucleotide sequence that is at least 90%, 91%, 92%, 93%, 94%, 95%, 96%, 97%, 98%, 99%, 99.5% or more identical to the nucleotide sequence set forth in SEQ ID NO: 11 and that hybridizes under stringent conditions to the complement of the nucleotide sequence set forth in SEQ ID NO: 11, and a nucleotide sequence that is at least 90%, 91%, 92%, 93%, 94%, 95%, 96%, 97%, 98%, 99%, 99.5% or more identical to the nucleotide sequence set forth in SEQ ID NO: 16 and that hybridizes under stringent conditions to the complement of the nucleotide sequence set forth in SEQ ID NO: 16;

(n) a nucleotide sequence that is at least 90%, 91%, 92%, 93%, 94%, 95%, 96%, 97%, 98%, 99%, 99.5% or more identical to the nucleotide sequence set forth in SEQ ID NO: 15 and that hybridizes under stringent conditions to the complement of the nucleotide sequence set forth in SEQ ID NO: 15, and a nucleotide sequence that is at least 90%, 91%, 92%, 93%, 94%, 95%, 96%, 97%, 98%, 99%, 99.5% or more identical to the nucleotide sequence set forth in SEQ ID NO: 16 and that hybridizes under stringent conditions to the complement of the nucleotide sequence set forth in SEQ ID NO: 16;

(o) a nucleotide sequence that is at least 90%, 91%, 92%, 93%, 94%, 95%, 96%, 97%, 98%, 99%, 99.5% or more identical to the nucleotide sequence set forth in SEQ ID NO: 15 and that hybridizes under stringent conditions to the complement of the nucleotide sequence set forth in SEQ ID NO: 15, and a nucleotide sequence that is at least 90%, 91%, 92%, 93%, 94%, 95%, 96%, 97%, 98%, 99%, 99.5% or more identical to the nucleotide sequence set forth in SEQ ID NO: 14 and that hybridizes under stringent conditions to the complement of the nucleotide sequence set forth in SEQ ID NO: 14;

(p) a nucleotide sequence that is at least 90%, 91%, 92%, 93%, 94%, 95%, 96%, 97%, 98%, 99%, 99.5% or more identical to the nucleotide sequence set forth in SEQ ID NO: 17 and that hybridizes under stringent conditions to the complement of the nucleotide sequence set forth in SEQ ID NO: 17, and a nucleotide sequence that is at least 90%, 91%, 92%, 93%, 94%, 95%, 96%, 97%, 98%, 99%, 99.5% or more identical to the nucleotide sequence set forth in SEQ ID NO: 18 and that hybridizes under stringent conditions to the complement of the nucleotide sequence set forth in SEQ ID NO: 18;

(q) a nucleotide sequence that is at least 90%, 91%, 92%, 93%, 94%, 95%, 96%, 97%, 98%, 99%, 99.5% or more identical to the nucleotide sequence set forth in SEQ ID NO: 19 and that hybridizes under stringent conditions to the complement of the nucleotide sequence set forth in SEQ ID NO: 19, and a nucleotide sequence that is at least 90%, 91%, 92%, 93%, 94%, 95%, 96%, 97%, 98%, 99%, 99.5% or more identical to the nucleotide sequence set forth in SEQ ID NO: 20 and that hybridizes under stringent conditions to the complement of the nucleotide sequence set forth in SEQ ID NO: 20;

(r) a nucleotide sequence that is at least 90%, 91%, 92%, 93%, 94%, 95%, 96%, 97%, 98%, 99%, 99.5% or more identical to the nucleotide sequence set forth in SEQ ID NO: 17 and that hybridizes under stringent conditions to the complement of the nucleotide sequence set forth in SEQ ID NO: 17, and a nucleotide sequence that is at least 90%, 91%, 92%, 93%, 94%, 95%, 96%, 97%, 98%, 99%, 99.5% or more identical to the nucleotide sequence set forth in SEQ ID NO: 22 and that hybridizes under stringent conditions to the complement of the nucleotide sequence set forth in SEQ ID NO: 22;

(s) a nucleotide sequence that is at least 90%, 91%, 92%, 93%, 94%, 95%, 96%, 97%, 98%, 99%, 99.5% or more identical to the nucleotide sequence set forth in SEQ ID NO: 21 and that hybridizes under stringent conditions to the complement of the nucleotide sequence set forth in SEQ ID NO: 21, and a nucleotide sequence that is at least 90%, 91%, 92%, 93%, 94%, 95%, 96%, 97%, 98%, 99%, 99.5% or more identical to the nucleotide sequence set forth in SEQ ID NO: 22 and that hybridizes under stringent conditions to the complement of the nucleotide sequence set forth in SEQ ID NO: 22; and

(t) a nucleotide sequence that is at least 90%, 91%, 92%, 93%, 94%, 95%, 96%, 97%, 98%, 99%, 99.5% or more identical to the nucleotide sequence set forth in SEQ ID NO: 21 and that hybridizes under stringent conditions to the complement of the nucleotide sequence set forth in SEQ ID NO: 21, and a nucleotide sequence that is at least 90%, 91%, 92%, 93%, 94%, 95%, 96%, 97%, 98%, 99%, 99.5% or more identical to the nucleotide sequence set forth in SEQ ID NO: 20 and that hybridizes under stringent conditions to the complement of the nucleotide sequence set forth in SEQ ID NO: 20.

The present invention extends to uses of nucleic acids, expression cassettes, vectors, bacteria, viruses, fungi, proteins, and amplification primers of the present invention, including, but not limited to, uses for controlling pests, uses for enhancing pest resistance in plants and plant parts, and uses for identifying, selecting and/or producing pest resistant plants.

In some embodiments, the use comprises introducing a nucleic acid of the present invention into a plant cell, growing the transgenic plant cell into a transgenic plant or plant part, and, optionally, selecting the transgenic plant or plant part based upon the presence of one or more pest resistant phenotypes (e.g., increased survival rate, increased growth rate, increased height and/or increased yield (e.g., increased biomass, increased seed yield, increased YGSMN, increased GMSTP, increased GWTPN, increased PYREC, decreased YRED, and/or decreased PB). Such uses may comprise transforming the plant cell with a transgenic bacterium/virus of the present invention.

In some embodiments, the use comprises culturing a transgenic bacterium/fungus comprising a nucleic acid of the present invention in/on a culture medium; isolating, from the culture medium, a pesticidal protein encoded by the nucleic acid; and applying the pesticidal protein to a plant or plant part, to an area surrounding a plant or plant part, to a pest, and/or to a pest's environment.

In some embodiments, the use comprises infecting a plant or plant part with a transgenic virus comprising a nucleic acid of the present invention.

In some embodiments, the use comprises applying a pesticidal protein of the present invention to a plant or plant part, to an area surrounding a plant or plant part, to a pest, and/or to a pest's environment.

The present invention also provides nonnaturally occurring plants and plant parts having enhanced pest resistance.

Plants and plant parts of the present invention may comprise any suitable exogenous nucleic acid(s). In some embodiments, the plant or plant part comprises at least one exogenous nucleic acid that encodes one or more proteins of the present invention and/or comprises, consists essentially of or consists of one or more nucleic acids of the present invention.

In some embodiments, the plant or plant part comprises within its genome an exogenous nucleic acid that comprises, consists essentially of or consists of:
(a) one or more of the nucleotide sequences set forth in SEQ ID NOs: 1-6;
(b) a nucleotide sequence that is at least about 70%, 75%, 80%, 85%, 90%, 91%, 92%, 93%, 94%, 95%, 96%, 97%, 98%, 99%, 99.5% or more identical to the nucleotide sequence set forth in SEQ ID NO: 1;
(c) a nucleotide sequence that is at least about 70%, 75%, 80%, 85%, 90%, 91%, 92%, 93%, 94%, 95%, 96%, 97%, 98%, 99%, 99.5% or more identical to the nucleotide sequence set forth in SEQ ID NO: 2;
(d) a nucleotide sequence that is at least about 70%, 75%, 80%, 85%, 90%, 91%, 92%, 93%, 94%, 95%, 96%, 97%, 98%, 99%, 99.5% or more identical to the nucleotide sequence set forth in SEQ ID NO: 3;
(e) a nucleotide sequence that is at least about 70%, 75%, 80%, 85%, 90%, 91%, 92%, 93%, 94%, 95%, 96%, 97%, 98%, 99%, 99.5% or more identical to the nucleotide sequence set forth in SEQ ID NO: 4;
(f) a nucleotide sequence that is at least about 70%, 75%, 80%, 85%, 90%, 91%, 92%, 93%, 94%, 95%, 96%, 97%, 98%, 99%, 99.5% or more identical to the nucleotide sequence set forth in SEQ ID NO: 5;
(g) a nucleotide sequence that is at least about 70%, 75%, 80%, 85%, 90%, 91%, 92%, 93%, 94%, 95%, 96%, 97%, 98%, 99%, 99.5% or more identical to the nucleotide sequence set forth in SEQ ID NO: 6;
(h) one or more nucleotide sequences that encode(s) a polypeptide comprising, consisting essentially of or consisting of the amino acid sequence set forth in any one of SEQ ID NOs: 7-10;
(i) a nucleotide sequence that encodes a polypeptide comprising, consisting essentially of or consisting of an amino acid sequence that is at least about 70%, 75%, 80%, 85%, 90%, 91%, 92%, 93%, 94%, 95%, 96%, 97%, 98%, 99%, 99.5% or more identical to the amino acid sequence set forth in SEQ ID NO: 7;
(j) a nucleotide sequence that encodes a polypeptide comprising, consisting essentially of or consisting of an amino acid sequence that is at least about 70%, 75%, 80%, 85%, 90%, 91%, 92%, 93%, 94%, 95%, 96%, 97%, 98%, 99%, 99.5% or more identical to the amino acid sequence set forth in SEQ ID NO: 8;
(k) a nucleotide sequence that encodes a polypeptide comprising, consisting essentially of or consisting of an amino acid sequence that is at least about 70%, 75%, 80%, 85%, 90%, 91%, 92%, 93%, 94%, 95%, 96%, 97%, 98%, 99%, 99.5% or more identical to the amino acid sequence set forth in SEQ ID NO: 9;
(l) a nucleotide sequence that encodes a polypeptide comprising, consisting essentially of or consisting of an amino acid sequence that is at least about 70%, 75%, 80%, 85%, 90%, 91%, 92%, 93%, 94%, 95%, 96%, 97%, 98%, 99%, 99.5% or more identical to the amino acid sequence set forth in SEQ ID NO: 10;
(m) a nucleotide sequence that is complementary to any one of the nucleotide sequences described in (a) to (1) above;
(n) a nucleotide sequence that hybridizes to any one of the nucleotide sequences described in (a) to (m) above under stringent hybridization conditions;
(o) a functional fragment of any one of the nucleotide sequences described in (a), (b), (d), (e), (g), (h), (i) and (k) above, wherein the functional fragment encodes a δ-endotoxin;
(p) a functional fragment of any one of the nucleotide sequences described in (a), (b), (d), (h) and (i) above, wherein the functional fragment encodes a polypeptide that comprises an N-terminal helical bundle domain that is at least about 70%, 75%, 80%, 85%, 90%, 91%, 92%, 93%, 94%, 95%, 96%, 97%, 98%, 99%, 99.5% or more identical to amino acids 72 to 286 of SEQ ID NO: 7, a central beta-sheet domain that is at least about 70%, 75%, 80%, 85%, 90%, 91%, 92%, 93%, 94%, 95%, 96%, 97%, 98%, 99%, 99.5% or more identical to amino acids 295 to 511 of SEQ ID NO: 7, and a C-terminal beta-sandwich domain that is at least about 70%, 75%, 80%, 85%, 90%, 91%, 92%, 93%, 94%, 95%, 96%, 97%, 98%, 99%, 99.5% or more identical to amino acids 514 to 675 of SEQ ID NO: 7;
(q) a functional fragment of any one of the nucleotide sequences described in (a), (e), (g), (h) and (k) above, wherein the functional fragment encodes a polypeptide that comprises an N-terminal helical bundle domain that is at least about 70%, 75%, 80%, 85%, 90%, 91%, 92%, 93%, 94%, 95%, 96%, 97%, 98%, 99%, 99.5% or more identical to amino acids 51 to 271 of SEQ ID NO: 9, a central beta-sheet domain that is at least about 70%, 75%, 80%, 85%, 90%, 91%, 92%, 93%, 94%, 95%, 96%, 97%, 98%, 99%, 99.5% or more identical to amino acids 279 to 481 of SEQ ID NO: 9, and a C-terminal beta-sandwich domain that is at least about 70%, 75%, 80%, 85%, 90%, 91%, 92%, 93%, 94%, 95%, 96%, 97%, 98%, 99%, 99.5% or more identical to amino acids 486 to 646 of SEQ ID NO: 9; and/or
(r) a functional fragment of any one of the nucleotide sequences described in (a), (c), (d), (f), (g), (h), (j) and (l) above, wherein the functional fragment encodes a protein the expression of which increases the expression, stability and/or activity of one or more δ-endotoxins. In some preferred embodiments, the exogenous nucleic acid comprises, consists essentially of or consists of a nucleotide sequence that encodes a protein having an amino acid sequence that is at least 70%, 75%, 80%, 85%, 90%, 91%, 92%, 93%, 94%, 95%, 96%, 97%, 98%, 99%, 99.5% or more identical to the amino acid sequence set forth in SEQ ID NO: 7 and a nucleotide sequence that encodes a protein having an amino acid sequence that is at least 70%, 75%, 80%, 85%, 90%, 91%, 92%, 93%, 94%, 95%, 96%, 97%, 98%, 99%, 99.5% or more identical to the amino acid sequence set forth in SEQ ID NO: 8. In some preferred embodiments, the exogenous nucleic acid comprises, consists essentially of or consists of a nucleotide sequence that encodes a protein having an amino acid sequence that is at least 70%, 75%, 80%, 85%, 90%, 91%, 92%, 93%, 94%, 95%, 96%, 97%, 98%, 99%, 99.5% or more identical to the amino acid sequence set forth in SEQ ID NO: 9 and a nucleotide sequence that encodes a protein having an amino acid sequence that is at least 70%, 75%, 80%, 85%, 90%, 91%, 92%, 93%, 94%, 95%, 96%, 97%, 98%, 99%, 99.5% or more identical to the amino acid sequence set forth in SEQ ID NO: 10.

In some embodiments, the exogenous nucleic acid comprises one or more constitutive promoters, tissue-specific promoters, chemically inducible promoters, wound-inducible promoters, stress-inducible promoters and developmental stage-specific promoters.

In some embodiments, the exogenous nucleic acid comprises one or more constitutive promoter sequences. For example, the exogenous nucleic acid may comprise one or more CaMV 19S, CaMV 35S, Arabidopsis At6669, maize H3 histone, rice actin 1, actin 2, rice cyclophilin, nos, Adh, sucrose synthase, pEMU, GOS2, constitutive root tip CT2, and/or ubiquitin (e.g., maize Ubi) promoter sequences. Thus, in some embodiments, the plant or plant part comprises an exogenous nucleic acid that comprises one or more of the nucleotide sequences described in (a) to (r) above operably linked to one or more constitutive promoter sequences.

In some embodiments, the exogenous nucleic acid comprises one or more tissue-specific or tissue-preferential promoter sequences. For example, the exogenous nucleic acid may comprise one or more leaf-, ligule-, node-, internode-, panicle-, root-, seed-, sheath-, stem-, and/or vascular bundle-specific promoter sequences. Thus, in some embodiments, the exogenous nucleic acid comprises one or more of the nucleotide sequences described in (a) to (r) above operably linked to one or more tissue-specific promoter sequences.

In some embodiments, the exogenous nucleic acid comprises one or more chemically inducible promoter sequences. Thus, in some embodiments, the exogenous nucleic acid comprises one or more of the nucleotide sequences described in (a) to (r) above operably linked to one or more chemically inducible promoter sequences.

In some embodiments, the exogenous nucleic acid comprises one or more wound-inducible promoter sequences. Thus, in some embodiments, the exogenous nucleic acid comprises one or more of the nucleotide sequences described in (a) to (r) above operably linked to one or more wound-inducible promoter sequences.

In some embodiments, the exogenous nucleic acid comprises one or more stress-inducible promoter sequences. For example, the exogenous nucleic acid may comprise one or more drought stress-inducible, osmotic stress-inducible, salt-inducible, temperature stress-inducible, and/or light stress-inducible promoter sequences. Thus, in some embodiments, the exogenous nucleic acid comprises one or more of the nucleotide sequences described in (a) to (r) above operably linked to one or more stress-inducible promoter sequences.

In some embodiments, the exogenous nucleic acid comprises one or more developmental stage-specific promoter sequences. For example, the exogenous nucleic acid may comprise a promoter sequence that drives expression prior to and/or during the seedling, tillering, panicle initiation, panicle differentiation, reproductive, and/or grain filling stage(s) of development. Thus, in some embodiments, the exogenous nucleic acid comprises one or more of the nucleotide sequences described in (a) to (r) above operably linked to one or more developmental stage-specific promoter sequences.

In some embodiments, the exogenous nucleic acid comprises one or more termination sequences. For example, the exogenous nucleic acid may comprise a termination sequence comprising a stop signal for RNA polymerase and a polyadenylation signal for polyadenylase. Thus, in some embodiments, the exogenous nucleic acid comprises one or more of the nucleotide sequences describes in (a) to (r) above operably linked to one or more termination sequences.

In some embodiments, the exogenous nucleic acid comprises one or more expression-enhancing sequence(s). For example, the exogenous nucleic acid may comprise one or more intron sequences (e.g., Adhl and/or bronzel) and/or viral leader sequences (from tobacco mosaic virus (TMV), tobacco etch virus (TEV), maize chlorotic mottle virus (MCMV), maize dwarf mottle virus (MDMV) or alfalfa mosaic virus (AMV), for example) that enhance expression of associated nucleotide sequences. Thus, in some embodiments, the exogenous nucleic acid comprises one or more of the nucleotide sequences described in (a) to (r) above operably linked to one or more expression-enhancing sequences.

In some embodiments, the exogenous nucleic acid comprises one or more transgenes that encodes a gene product that provides enhanced abiotic stress tolerance (e.g., drought stress tolerance, osmotic stress tolerance, salt stress tolerance and/or temperature stress tolerance), herbicide-resistance (e.g., glyphosate-, Sulfonylurea-, imidazolinione-, dicamba-, glufisinate-, phenoxy proprionic acid-, cycloshexome-, traizine-, benzonitrile-, and/or broxynil-resistance), pest-resistance (e.g., Acarina-, bacterial-, fungal, gastropod-, insect-, nematode-, oomycete-, phytoplasma-, protozoa-, and/or viral-resistance) and/or disease-resistance.

In some embodiments, the exogenous nucleic acid has been codon optimized for expression in plants. In some such embodiments, the exogenous nucleic acid has been optimized for expression in the particular species of interest (i.e., for expression in the species of plant into which has been introduced).

Plants and plant parts of the present invention may exhibit a variety of pest resistant phenotypes, including, but not limited to, increased survival rate, increased growth rate, increased height and/or increased yield (e.g., increased biomass, increased seed yield, increased YGSMN, increased GMSTP, increased GWTPN, increased PYREC, decreased YRED, and/or decreased PB) when grown under pest stress conditions (e.g., Acarina and/or insect infestation). In some embodiments, one or more pest resistant phenotypes is increased by at least about 5%, 10%, 15%, 20%, 25%, 30%, 35%, 40%, 45%, 50%, 55%, 60%, 65%, 75%, 80%, 85%, 90%, 95%, 100%, 125%, 150%, 175%, 200%, 250%, 300%, or more as compared to a control plant or plant part (e.g., a native plant of the same species) when each is grown under the same (or substantially the same) environmental conditions.

In some embodiments, the yield (e.g., seed yield, biomass, GWTPN, PYREC and/or YGSMN) of the plant or plant part is increased by at least about 5%, 10%, 15%, 20%, 25%, 30%, 35%, 40%, 45%, 50%, 55%, 60%, 65%, 75%, 80%, 85%, 90%, 95%, 100%, 125%, 150%, 175%, 200%, 250%, 300% or more as compared to a control plant or plant part (e.g., a native plant of the same species) grown under the same (or substantially the same) environmental conditions. For example, the seed yield and/or biomass of the plant or plant part may be increased by at least about 5%, 10%, 15%, 20%, 25%, 30%, 35%, 40%, 45%, 50%, 55%, 60%, 65%, 75%, 80%, 85%, 90%, 95%, 100%, 125%, 150%, 175%, 200%, 250%, 300% or more as compared to a control plant or plant part grown under the same (or substantially the same) pest stress conditions.

In some embodiments, the expression, stability and/or activity of one or more heterologous endotoxins in the plant or plant part is increased by at least about 5%, 10%, 15%, 20%, 25%, 30%, 35%, 40%, 45%, 50%, 55%, 60%, 65%, 75%, 80%, 85%, 90%, 95%, 100%, 125%, 150%, 175%, 200%, 250%, 300% or more as compared to a control plant (e.g., a native plant of the same species) grown under the same (or substantially the same) environmental conditions. For example, the expression, stability and/or activity of one or more CRY71 proteins and/or one or more CRY72 proteins may be increased by at least about 5%, 10%, 15%, 20%, 25%, 30%, 35%, 40%, 45%, 50%, 55%, 60%, 65%, 75%, 80%, 85%, 90%, 95%, 100%, 125%, 150%, 175%, 200%, 250%, 300% or more in a plant or plant part comprising an exogenous nucleic acid encoding SEQ ID NO: 8 and/or SEQ ID NO: 10.

In some embodiments, the pest resistance (e.g., Acarina-, Anoplura-, Coleoptera-, Dermaptera-, Diptera-, Hemiptera-, Heteroptera-, Homoptera-, Hymenoptera-, Isoptera-, Lepidoptera-, Mallophaga-, Orthoptera-, Psocoptera-, Siphoptera-, Thysanoptera-, Thysanura-, and/or Trichoptera-resistance) of the plant or plant part is increased by at least about 5%, 10%, 15%, 20%, 25%, 30%, 35%, 40%, 45%, 50%, 55%, 60%, 65%, 75%, 80%, 85%, 90%, 95%, 100%, 125%, 150%, 175%, 200%, 250%, 300% or more as compared to a control plant (e.g., a native plant of the same species) grown under the same (or substantially the same) environmental conditions.

Plants and plant parts of the present invention may be of any suitable plant type, including, but not limited to, plants belonging to the superfamily Viridiplantae. In some embodiments the plant or plant part is a fodder crop, a food crop, an ornamental plant, a tree or a shrub. For example, in some embodiments, the plant or plant part is a variety of Acer spp., Actinidia spp., Abelmoschus spp., Agropyron spp., Album spp., Amaranthus spp., Ananas comosus, Annona spp., Apium graveolens, Arachis spp, Artocarpus spp., Asparagus officinalis, Avena spp. (e.g. Avena sativa, Avena fatua, Avena byzantina, Avena fatua var. sativa, Avena hybrida), Averrhoa carambola, Benincasa hispida, Bertholletia excelsea, Beta vulgaris, Brassica spp. (e.g. Brassica napus, Brassica rapa ssp. [canola, oilseed rape, turnip rape]), Cadaba farinosa, Camellia sinensis, Canna indica, Capsicum spp., Carex elata, Carica papaya, Carissa macrocarpa, Carya spp., Carthamus tinctorius, Castanea spp., Cichorium endivia, Cinnamomum spp., Citrullus lanatus, Citrus spp., Cocos spp., Coffea spp., Colocasia esculenta, Cola spp., Coriandrum sativum, Corylus spp., Crataegus spp., Crocus sativus, Cucurbita spp., Cucumis spp., Cynara spp., Daucus carota, Desmodium spp., Dimocarpus longan, Dioscorea spp., Diospyros spp., Echinochloa spp., Elaeis (e.g. Elaeis guineensis, Elaeis oleifera), Eleusine coracana, Eriobotrya japonica, Eugenia uniflora, Fagopyrum spp., Fagus spp., Ficus carica, Fortunella spp., Fragaria spp., Ginkgo biloba, Glycine spp. (e.g. Glycine max, Soja hispida or Soja max), Gossypium hirsutum, Helianthus spp. (e.g. Helianthus annuus), Hemerocallis fulva, Hibiscus spp., Hordeum spp. (e.g. Hordeum vulgare), Ipomoea batatas, Juglans spp., Lactuca sativa, Lathyrus spp., Lens culinaris, Linum usitatissimum, Litchi chinensis, Lotus spp., Luffa acutangula, Lupinus spp., Luzula sylvatica, Lycopersicon spp. (e.g. Lycopersicon esculentum, Lycopersicon lycopersicum, Lycopersicon pyriforme), Macrotyloma spp., Malus spp., Malpighia emarginata, Mammea americana, Mangifera indica, Manihot spp., Manilkara zapota, Medicago sativa, Melilotus spp., Mentha spp., Miscanthus spp., Momordica spp., Morus nigra, Musa spp., Nicotiana spp., Olea spp., Opuntia spp., Ornithopus spp., Oryza spp. (e.g. Oryza sativa, Oryza latifolia), Panicum miliaceum, Passiflora edulis, Pastinaca sativa, Persea spp., Petroselinum crispum, Phaseolus spp., Phoenix spp., Physalis spp., Pinus spp., Pistacia vera, Pisum spp., Poa spp., Populus spp., Prosopis spp., Prunus spp., Psidium spp., Punica granatum, Pyrus communis, Quercus spp., Raphanus sativus, Rheum rhabarbarum, Ribes spp., Ricinus communis, Rubus spp., Saccharum spp., Sambucus spp., Secale cereale, Sesamum spp., Sinapis sp., Solanum spp. (e.g. Solanum tuberosum, Solanum integrifolium or Solanum lycopersicum), Sorghum bicolor, Spinacia spp., Syzygium spp., Tagetes spp., Tamarindus indica, Theobroma cacao, Trifolium spp., Triticosecale rimpaui, Triticum spp. (e.g. Triticum aestivum, Triticum durum, Triticum turgidum, Triticum hybernum, Triticum macha, Triticum sativum or Triticum vulgare), Tropaeolum minus, Tropaeolum majus, Vaccinium spp., Vicia spp., Vigna spp., Viola odorata, Vitis spp., Zea mays, Zizania palustris or Ziziphus spp., amongst others. In some embodiments, the plant or plant part is a rice, maize, wheat, barley, sorghum, millet, oat, triticale, rye, buckwheat, fonio, quina, sugar cane, bamboo, banana, ginger, onion, lily, daffodil, iris, amaryllis, orchid, canna, bluebell, tulip, garlic, secale, einkorn, spelt, emmer, durum, kamut, grass (e.g., gramma grass), teff, milo, flax, Tripsacum sp., or teosinte plant or plant part. In some embodiments, the plant or plant part is a blackberry, raspberry, strawberry, barberry, bearberry, blueberry, coffee berry, cranberry, crowberry, currant, elderberry, gooseberry, goji berry, honeyberry, lemon, lime, lingonberry, mangosteen, orange, pepper, persimmon, pomegranate, prune, cotton, clover, acai, plum, peach, nectarin, cherry, guava, almond, pecan, walnut, apple, amaranth, sweet pea, pear, potato, soybean, sugar beet, sunflower, sweet potato, tamarind, tea, tobacco or tomato plant or plant part.

Plants and plant parts of the present invention may be produced using any suitable method, including, but not limited to, methods of the present invention.

The present invention extends to products harvested from plants and plant parts of the present invention, including, but not limited to, plant cells and harvestable plant parts such as seeds, leaves, fruits, flowers, stems, rhizomes, tubers and bulbs. In some embodiments, the harvested product is a plant part capable of producing a plant or plant part that expresses one or more CRY71 proteins and/or one or more CRY72 proteins, and/or that exhibits enhanced pest resistance (e.g., enhanced Acarina and/or insect resistance). In some embodiments, the harvested product is a plant part capable of producing a plant or plant that exhibits increased survival rate, increased growth rate, increased height and/or increased yield (e.g., increased biomass, increased seed yield, increased YGSMN, increased GWTPN, increased PYREC, and/or decreased YRED) when grown under pest stress conditions (e.g., Acarina stress conditions and/or insect stress conditions).

The present invention also extends to products derived from harvestable plant parts, including, but not limited to, dry pellets and powders, oils, fats, fatty acids, starches and proteins.

The present invention also encompasses methods of enhancing pest resistance (e.g., Acarina-, Anoplura-, Coleoptera-, Dermaptera-, Diptera-, Hemiptera-, Heteroptera-, Homoptera-, Hymenoptera-, Isoptera-, Lepidoptera-, Mallophaga-, Orthoptera-, Psocoptera-, Siphoptera-, Thysanoptera-, Thysanura-, and/or Trichoptera-resistance) in a plant or plant part.

Pest resistance may be enhanced by increasing the expression, stability and/or activity of one or more CRY71 proteins and/or one or more CRY72 proteins. Thus, methods of enhancing pest resistance in a plant or plant part may comprise, consist essentially of or consist of increasing the expression, stability and/or activity of one or more CRY71 proteins, one or more CRY72 proteins and/or one or more ORF2 proteins in the plant or plant part.

The expression, stability and/or activity of CRY71 proteins may be increased via any suitable method, including, but not limited to, expression of exogenous CRY71 proteins, overexpression of one or more CRY71 precursors, down-regulation and/or inhibition of one or more CRY71 inhibitors, overexpression of one or more enzymes involved in CRY71 synthesis, and expression of one or more exogenous enzymes involved in CRY71 synthesis. In some embodiments, the expression, stability and/or activity of one or more CRY71 proteins is increased by:
(a) increasing the expression and/or activity of one or more exogenous CRY71 proteins in the plant or plant part;
(b) increasing the expression and/or activity of one or more CRY71 protein precursors in the plant or plant part;
(c) increasing the expression and/or activity of one or more CRY71 chaperones in the plant or plant part;
(d) decreasing the expression and/or activity of one or more CRY71 protein inhibitors in the plant or plant part;
(e) increasing the expression and/or activity of one or more enzymes involved in CRY71 protein synthesis in the plant or plant part; and/or
(f) increasing the expression and/or activity of one or more exogenous enzymes involved in CRY71 protein synthesis in the plant or plant part.

The expression and/or activity of CRY72 proteins may be increased via any suitable method, including, but not limited to, expression of exogenous CRY72 proteins, overexpression of one or more CRY72 precursors, down-regulation and/or inhibition of one or more CRY72 inhibitors, overexpression of one or more enzymes involved in CRY72 synthesis, and expression of one or more exogenous enzymes involved in CRY72 synthesis. In some embodiments, the expression and/or activity of one or more CRY72 proteins is/are increased by:
(a) increasing the expression and/or activity of one or more exogenous CRY72 proteins in the plant or plant part;
(b) increasing the expression and/or activity of one or more CRY72 protein precursors in the plant or plant part;
(c) increasing the expression and/or activity of one or more CRY72 chaperones in the plant or plant part;
(d) decreasing the expression and/or activity of one or more CRY72 protein inhibitors in the plant or plant part;
(e) increasing the expression and/or activity of one or more enzymes involved in CRY72 protein synthesis in the plant or plant part; and/or
(f) increasing the expression and/or activity of one or more exogenous enzymes involved in CRY72 protein synthesis in the plant or plant part.

Thus, in some embodiments, pest resistance may be enhanced by introducing/expressing an exogenous nucleic acid comprising:
(a) one or more of the nucleotide sequences set forth in SEQ ID NOs: 1-6;
(b) a nucleotide sequence that is at least about 70%, 75%, 80%, 85%, 90%, 91%, 92%, 93%, 94%, 95%, 96%, 97%, 98%, 99%, 99.5% or more identical to the nucleotide sequence set forth in SEQ ID NO: 1;
(c) a nucleotide sequence that is at least about 70%, 75%, 80%, 85%, 90%, 91%, 92%, 93%, 94%, 95%, 96%, 97%, 98%, 99%, 99.5% or more identical to the nucleotide sequence set forth in SEQ ID NO: 2;
(d) a nucleotide sequence that is at least about 70%, 75%, 80%, 85%, 90%, 91%, 92%, 93%, 94%, 95%, 96%, 97%, 98%, 99%, 99.5% or more identical to the nucleotide sequence set forth in SEQ ID NO: 3;
(e) a nucleotide sequence that is at least about 70%, 75%, 80%, 85%, 90%, 91%, 92%, 93%, 94%, 95%, 96%, 97%, 98%, 99%, 99.5% or more identical to the nucleotide sequence set forth in SEQ ID NO: 4;
(f) a nucleotide sequence that is at least about 70%, 75%, 80%, 85%, 90%, 91%, 92%, 93%, 94%, 95%, 96%, 97%, 98%, 99%, 99.5% or more identical to the nucleotide sequence set forth in SEQ ID NO: 5;
(g) a nucleotide sequence that is at least about 70%, 75%, 80%, 85%, 90%, 91%, 92%, 93%, 94%, 95%, 96%, 97%, 98%, 99%, 99.5% or more identical to the nucleotide sequence set forth in SEQ ID NO: 6;
(h) one or more nucleotide sequences that encodes a polypeptide comprising, consisting essentially of or consisting of the amino acid sequence set forth in any one of SEQ ID NOs: 7-10;
(i) a nucleotide sequence that encodes a polypeptide comprising, consisting essentially of or consisting of an amino acid sequence that is at least about 70%, 75%, 80%, 85%, 90%, 91%, 92%, 93%, 94%, 95%, 96%, 97%, 98%, 99%, 99.5% or more identical to the amino acid sequence set forth in SEQ ID NO: 7;
(j) a nucleotide sequence that encodes a polypeptide comprising, consisting essentially of or consisting of an amino acid sequence that is at least about 70%, 75%, 80%, 85%, 90%, 91%, 92%, 93%, 94%, 95%, 96%, 97%, 98%, 99%, 99.5% or more identical to the amino acid sequence set forth in SEQ ID NO: 8;
(k) a nucleotide sequence that encodes a polypeptide comprising, consisting essentially of or consisting of an amino acid sequence that is at least about 70%, 75%, 80%, 85%, 90%, 91%, 92%, 93%, 94%, 95%, 96%, 97%, 98%, 99%, 99.5% or more identical to the amino acid sequence set forth in SEQ ID NO: 9;
(l) a nucleotide sequence that encodes a polypeptide comprising, consisting essentially of or consisting of an amino acid sequence that is at least about 70%, 75%, 80%, 85%, 90%, 91%, 92%, 93%, 94%, 95%, 96%, 97%, 98%, 99%, 99.5% or more identical to the amino acid sequence set forth in SEQ ID NO: 10;
(m) a nucleotide sequence that is complementary to any one of the nucleotide sequences described in (a) to (l) above;
(n) a nucleotide sequence that hybridizes to any one of the nucleotide sequences described in (a) to (m) above under stringent hybridization conditions;
(o) a functional fragment of any one of the nucleotide sequences described in (a), (b), (d), (e), (g), (h), (i) and (k) above, wherein the functional fragment encodes a δ-endotoxin;
(p) a functional fragment of any one of the nucleotide sequences described in (a), (b), (d), (h) and (i) above, wherein the functional fragment encodes a polypeptide that comprises an N-terminal helical bundle domain that is at least about 70%, 75%, 80%, 85%, 90%, 91%, 92%, 93%, 94%, 95%, 96%, 97%, 98%, 99%, 99.5% or more identical to amino acids 72 to 286 of SEQ ID NO: 7, a central beta-sheet domain that is at least about 70%, 75%, 80%, 85%, 90%, 91%, 92%, 93%, 94%, 95%, 96%, 97%, 98%, 99%, 99.5% or more identical to amino acids 295 to 511 of SEQ ID NO: 7, and a C-terminal beta-sandwich domain that is at least about 70%, 75%, 80%, 85%, 90%, 91%, 92%, 93%, 94%, 95%, 96%, 97%, 98%, 99%, 99.5% or more identical to amino acids 514 to 675 of SEQ ID NO: 7;
(q) a functional fragment of any one of the nucleotide sequences described in (a), (e), (g), (h) and (k) above, wherein the functional fragment encodes a polypeptide that comprises an N-terminal helical bundle domain that is at least about 70%, 75%, 80%, 85%, 90%, 91%, 92%, 93%, 94%, 95%, 96%, 97%, 98%, 99%, 99.5% or more identical to amino acids 51 to 271 of SEQ ID NO: 9, a central beta-sheet domain that is at least about 70%, 75%, 80%, 85%, 90%, 91%, 92%, 93%, 94%, 95%, 96%, 97%, 98%, 99%, 99.5% or more identical to amino acids 279 to 481 of SEQ ID NO: 9, and a C-terminal beta-sandwich domain that is at least about 70%, 75%, 80%, 85%, 90%, 91%, 92%, 93%, 94%, 95%, 96%, 97%, 98%, 99%, 99.5% or more identical to amino acids 486 to 646 of SEQ ID NO: 9; and/or
(r) a functional fragment of any one of the nucleotide sequences described in (a), (c), (d), (f), (g), (h), (j) and (l) above, wherein the functional fragment encodes a protein the expression of which increases the expression, stability and/or activity of one or more δ-endotoxins.

The present invention also encompasses methods of identifying, selecting and/or producing a plant or plant part having enhanced pest resistance (e.g., Acarina-, Anoplura-, Coleoptera-, Dermaptera-, Diptera-, Hemiptera-, Heteroptera-, Homoptera-, Hymenoptera-, Isoptera-, Lepidoptera-, Mallophaga-, Orthoptera-, Psocoptera-, Siphoptera-, Thysanoptera-, Thysanura-, and/or Trichoptera-resistance).

Methods of identifying plants and plant parts having enhanced pest resistance may comprise, consist essentially of or consist of detecting, in the plant or plant part, a nucleic acid (e.g., an exogenous nucleic acid) comprising:
(a) one or more of the nucleotide sequences set forth in SEQ ID NOs: 1-6;
(b) a nucleotide sequence that is at least about 70%, 75%, 80%, 85%, 90%, 91%, 92%, 93%, 94%, 95%, 96%, 97%, 98%, 99%, 99.5% or more identical to the nucleotide sequence set forth in SEQ ID NO: 1;
(c) a nucleotide sequence that is at least about 70%, 75%, 80%, 85%, 90%, 91%, 92%, 93%, 94%, 95%, 96%, 97%, 98%, 99%, 99.5% or more identical to the nucleotide sequence set forth in SEQ ID NO: 2;
(d) a nucleotide sequence that is at least about 70%, 75%, 80%, 85%, 90%, 91%, 92%, 93%, 94%, 95%, 96%, 97%, 98%, 99%, 99.5% or more identical to the nucleotide sequence set forth in SEQ ID NO: 3;
(e) a nucleotide sequence that is at least about 70%, 75%, 80%, 85%, 90%, 91%, 92%, 93%, 94%, 95%, 96%, 97%, 98%, 99%, 99.5% or more identical to the nucleotide sequence set forth in SEQ ID NO: 4;
(f) a nucleotide sequence that is at least about 70%, 75%, 80%, 85%, 90%, 91%, 92%, 93%, 94%, 95%, 96%, 97%, 98%, 99%, 99.5% or more identical to the nucleotide sequence set forth in SEQ ID NO: 5;
(g) a nucleotide sequence that is at least about 70%, 75%, 80%, 85%, 90%, 91%, 92%, 93%, 94%, 95%, 96%, 97%, 98%, 99%, 99.5% or more identical to the nucleotide sequence set forth in SEQ ID NO: 6;
(h) one or more nucleotide sequences that encodes a polypeptide comprising, consisting essentially of or consisting of the amino acid sequence set forth in any one of SEQ ID NOs: 7-10;
(i) a nucleotide sequence that encodes a polypeptide comprising, consisting essentially of or consisting of an amino acid sequence that is at least about 70%, 75%, 80%, 85%, 90%, 91%, 92%, 93%, 94%, 95%, 96%, 97%, 98%, 99%, 99.5% or more identical to the amino acid sequence set forth in SEQ ID NO: 7;
(j) a nucleotide sequence that encodes a polypeptide comprising, consisting essentially of or consisting of an amino acid sequence that is at least about 70%, 75%, 80%, 85%, 90%, 91%, 92%, 93%, 94%, 95%, 96%, 97%, 98%, 99%, 99.5% or more identical to the amino acid sequence set forth in SEQ ID NO: 8;
(k) a nucleotide sequence that encodes a polypeptide comprising, consisting essentially of or consisting of an amino acid sequence that is at least about 70%, 75%, 80%, 85%, 90%, 91%, 92%, 93%, 94%, 95%, 96%, 97%, 98%, 99%, 99.5% or more identical to the amino acid sequence set forth in SEQ ID NO: 9;
(l) a nucleotide sequence that encodes a polypeptide comprising, consisting essentially of or consisting of an amino acid sequence that is at least about 70%, 75%, 80%, 85%, 90%, 91%, 92%, 93%, 94%, 95%, 96%, 97%, 98%, 99%, 99.5% or more identical to the amino acid sequence set forth in SEQ ID NO: 10;
(m) a nucleotide sequence that is complementary to any one of the nucleotide sequences described in (a) to (l) above;
(n) a nucleotide sequence that hybridizes to any one of the nucleotide sequences described in (a) to (m) above under stringent hybridization conditions;
(o) a functional fragment of any one of the nucleotide sequences described in (a), (b), (d), (e), (g), (h), (i) and (k) above, wherein the functional fragment encodes a δ-endotoxin;
(p) a functional fragment of any one of the nucleotide sequences described in (a), (b), (d), (h) and (i) above, wherein the functional fragment encodes a polypeptide that comprises an N-terminal helical bundle domain that is at least about 70%, 75%, 80%, 85%, 90%, 91%, 92%, 93%, 94%, 95%, 96%, 97%, 98%, 99%, 99.5% or more identical to amino acids 72 to 286 of SEQ ID NO: 7, a central beta-sheet domain that is at least about 70%, 75%, 80%, 85%, 90%, 91%, 92%, 93%, 94%, 95%, 96%, 97%, 98%, 99%, 99.5% or more identical to amino acids 295 to 511 of SEQ ID NO: 7, and a C-terminal beta-sandwich domain that is at least about 70%, 75%, 80%, 85%, 90%, 91%, 92%, 93%, 94%, 95%, 96%, 97%, 98%, 99%, 99.5% or more identical to amino acids 514 to 675 of SEQ ID NO: 7;
(q) a functional fragment of any one of the nucleotide sequences described in (a), (e), (g), (h) and (k) above, wherein the functional fragment encodes a polypeptide that comprises an N-terminal helical bundle domain that is at least about 70%, 75%, 80%, 85%, 90%, 91%, 92%, 93%, 94%, 95%, 96%, 97%, 98%, 99%, 99.5% or more identical to amino acids 51 to 271 of SEQ ID NO: 9, a central beta-sheet domain that is at least about 70%, 75%, 80%, 85%, 90%, 91%, 92%, 93%, 94%, 95%, 96%, 97%, 98%, 99%, 99.5% or more identical to amino acids 279 to 481 of SEQ ID NO: 9, and a C-terminal beta-sandwich domain that is at least about 70%, 75%, 80%, 85%, 90%, 91%, 92%, 93%, 94%, 95%, 96%, 97%, 98%, 99%, 99.5% or more identical to amino acids 486 to 646 of SEQ ID NO: 9; and/or
(r) a functional fragment of any one of the nucleotide sequences described in (a), (c), (d), (f), (g), (h), (j) and (l) above, wherein the functional fragment encodes a protein the expression of which increases the expression, stability and/or activity of one or more δ-endotoxins.

Methods of producing plants and plant parts having enhanced pest resistance may comprise, consist essentially of or consist of:
(a) detecting, in a plant part, the presence of an exogenous nucleic acid encoding one or more CRY71 proteins, one or more CRY72 proteins and/or one or more ORF2 proteins (e.g., a nucleic acid comprising a nucleotide sequence that is at least 70%, 75%, 80%, 85%, 90%, 91%, 92%, 93%, 94%, 95%, 96%, 97%, 98%, 99%, 99.5% or more identical to the nucleotide sequence set forth in any one of SEQ ID NOs: 1-6), and producing a plant from the plant part;
(b) introducing, into a plant part, an exogenous nucleic acid encoding one or more CRY71 proteins, one or more CRY72 proteins and/or one or more ORF2 proteins (e.g., an exogenous nucleic acid comprising a nucleotide sequence that is at least 70%, 75%, 80%, 85%, 90%, 91%, 92%, 93%, 94%, 95%, 96%, 97%, 98%, 99%, 99.5% or more identical to the nucleotide sequence set forth in any one of SEQ ID NOs: 1-6), and growing the plant part into a plant; such methods may further comprise detecting the exogenous nucleic acid in the plant part and/or in the plant produced from the plant part;
(c) introducing, into a plant part, an exogenous nucleic acid encoding one or more CRY71 proteins, one or more CRY72 proteins and/or one or more ORF2 proteins (e.g., an exogenous nucleic acid comprising a nucleotide sequence that is at least 70%, 75%, 80%, 85%, 90%, 91%, 92%, 93%, 94%, 95%, 96%, 97%, 98%, 99%, 99.5% or more identical to the nucleotide sequence set forth in any one of SEQ ID NOs: 1-6), detecting the presence of the exogenous nucleic acid in the plant part, and growing the plant part into a plant;
(d) crossing a first parent plant or plant part with a second parent plant or plant part, wherein the first parent plant or plant part comprises within its genome a nucleic acid (e.g., an exogenous nucleic acid) encoding one or more CRY71 proteins, one or more CRY72 proteins and/or one or more ORF2 proteins (e.g., an exogenous nucleic acid comprising a nucleotide sequence that is at least 70%, 75%, 80%, 85%, 90%, 91%, 92%, 93%, 94%, 95%, 96%, 97%, 98%, 99%, 99.5% or more identical to the nucleotide sequence set forth in any one of SEQ ID NOs: 1-6); and/or
(e) introgressing an exogenous nucleic acid encoding one or more CRY71 proteins, one or more CRY72 proteins and/or one or more ORF2 proteins (e.g., an exogenous nucleic acid comprising a nucleotide sequence that is at least 70%, 75%, 80%, 85%, 90%, 91%, 92%, 93%, 94%, 95%, 96%, 97%, 98%, 99%, 99.5% or more identical to the nucleotide sequence set forth in any one of SEQ ID NOs: 1-6) into a plant or plant part lacking the exogenous nucleic acid.

In some embodiments, methods of producing plants having enhanced pest resistance comprise, consist essentially of or consist of detecting, in a plant or plant part, the presence of a nucleic acid (e.g., an exogenous nucleic acid) comprising, consisting essentially of or consisting of:
(a) one or more of the nucleotide sequences set forth in SEQ ID NOs: 1-6;
(b) a nucleotide sequence that is at least about 70%, 75%, 80%, 85%, 90%, 91%, 92%, 93%, 94%, 95%, 96%, 97%, 98%, 99%, 99.5% or more identical to the nucleotide sequence set forth in SEQ ID NO: 1;
(c) a nucleotide sequence that is at least about 70%, 75%, 80%, 85%, 90%, 91%, 92%, 93%, 94%, 95%, 96%, 97%, 98%, 99%, 99.5% or more identical to the nucleotide sequence set forth in SEQ ID NO: 2;
(d) a nucleotide sequence that is at least about 70%, 75%, 80%, 85%, 90%, 91%, 92%, 93%, 94%, 95%, 96%, 97%, 98%, 99%, 99.5% or more identical to the nucleotide sequence set forth in SEQ ID NO: 3;
(e) a nucleotide sequence that is at least about 70%, 75%, 80%, 85%, 90%, 91%, 92%, 93%, 94%, 95%, 96%, 97%, 98%, 99%, 99.5% or more identical to the nucleotide sequence set forth in SEQ ID NO: 4;
(f) a nucleotide sequence that is at least about 70%, 75%, 80%, 85%, 90%, 91%, 92%, 93%, 94%, 95%, 96%, 97%, 98%, 99%, 99.5% or more identical to the nucleotide sequence set forth in SEQ ID NO: 5;
(g) a nucleotide sequence that is at least about 70%, 75%, 80%, 85%, 90%, 91%, 92%, 93%, 94%, 95%, 96%, 97%, 98%, 99%, 99.5% or more identical to the nucleotide sequence set forth in SEQ ID NO: 6;
(h) one or more nucleotide sequences that encodes a polypeptide comprising, consisting essentially of or consisting of the amino acid sequence set forth in any one of SEQ ID NOs: 7-10;
(i) a nucleotide sequence that encodes a polypeptide comprising, consisting essentially of or consisting of an amino acid sequence that is at least about 70%, 75%, 80%, 85%, 90%, 91%, 92%, 93%, 94%, 95%, 96%, 97%, 98%, 99%, 99.5% or more identical to the amino acid sequence set forth in SEQ ID NO: 7;
(j) a nucleotide sequence that encodes a polypeptide comprising, consisting essentially of or consisting of an amino acid sequence that is at least about 70%, 75%, 80%, 85%, 90%, 91%, 92%, 93%, 94%, 95%, 96%, 97%, 98%, 99%, 99.5% or more identical to the amino acid sequence set forth in SEQ ID NO: 8;
(k) a nucleotide sequence that encodes a polypeptide comprising, consisting essentially of or consisting of an amino acid sequence that is at least about 70%, 75%, 80%, 85%, 90%, 91%, 92%, 93%, 94%, 95%, 96%, 97%, 98%, 99%, 99.5% or more identical to the amino acid sequence set forth in SEQ ID NO: 9;
(l) a nucleotide sequence that encodes a polypeptide comprising, consisting essentially of or consisting of an amino acid sequence that is at least about 70%, 75%, 80%, 85%, 90%, 91%, 92%, 93%, 94%, 95%, 96%, 97%, 98%, 99%, 99.5% or more identical to the amino acid sequence set forth in SEQ ID NO: 10;
(m) a nucleotide sequence that is complementary to any one of the nucleotide sequences described in (a) to (l) above;
(n) a nucleotide sequence that hybridizes to any one of the nucleotide sequences described in (a) to (m) above under stringent hybridization conditions;
(o) a functional fragment of any one of the nucleotide sequences described in (a), (b), (d), (e), (g), (h), (i) and (k) above, wherein the functional fragment encodes a δ-endotoxin;
(p) a functional fragment of any one of the nucleotide sequences described in (a), (b), (d), (h) and (i) above, wherein the functional fragment encodes a polypeptide that comprises an N-terminal helical bundle domain that is at least about 70%, 75%, 80%, 85%, 90%, 91%, 92%, 93%, 94%, 95%, 96%, 97%, 98%, 99%, 99.5% or more identical to amino acids 72 to 286 of SEQ ID NO: 7, a central beta-sheet domain that is at least about 70%, 75%, 80%, 85%, 90%, 91%, 92%, 93%, 94%, 95%, 96%, 97%, 98%, 99%, 99.5% or more identical to amino acids 295 to 511 of SEQ ID NO: 7, and a C-terminal beta-sandwich domain that is at least about 70%, 75%, 80%, 85%, 90%, 91%, 92%, 93%, 94%, 95%, 96%, 97%, 98%, 99%, 99.5% or more identical to amino acids 514 to 675 of SEQ ID NO: 7;
(q) a functional fragment of any one of the nucleotide sequences described in (a), (e), (g), (h) and (k) above, wherein the functional fragment encodes a polypeptide that comprises an N-terminal helical bundle domain that is at least about 70%, 75%, 80%, 85%, 90%, 91%, 92%, 93%, 94%, 95%, 96%, 97%, 98%, 99%, 99.5% or more identical to amino acids 51 to 271 of SEQ ID NO: 9, a central beta-sheet domain that is at least about 70%, 75%, 80%, 85%, 90%, 91%, 92%, 93%, 94%, 95%, 96%, 97%, 98%, 99%, 99.5% or more identical to amino acids 279 to 481 of SEQ ID NO: 9, and a C-terminal beta-sandwich domain that is at least about 70%, 75%, 80%, 85%, 90%, 91%, 92%, 93%, 94%, 95%, 96%, 97%, 98%, 99%, 99.5% or more identical to amino acids 486 to 646 of SEQ ID NO: 9; and/or
(r) a functional fragment of any one of the nucleotide sequences described in (a), (c), (d), (f), (g), (h), (j) and (l) above, wherein the functional fragment encodes a protein the expression of which increases the expression, stability and/or activity of one or more δ-endotoxins; and
   producing a plant from the plant or plant part, wherein the plant so produced comprises the nucleic acid (or a functional fragment thereof) and exhibits enhanced pest resistance as compared to a control plant of the same species grown under the same environmental conditions.

In some embodiments, methods of producing plants having enhanced pest resistance comprise, consist essentially of or consist of introducing, into a plant or plant part, an exogenous nucleic acid comprising, consisting essentially of or consisting of:
(a) one or more of the nucleotide sequences set forth in SEQ ID NOs: 1-6;
(b) a nucleotide sequence that is at least about 70%, 75%, 80%, 85%, 90%, 91%, 92%, 93%, 94%, 95%, 96%, 97%, 98%, 99%, 99.5% or more identical to the nucleotide sequence set forth in SEQ ID NO: 1;
(c) a nucleotide sequence that is at least about 70%, 75%, 80%, 85%, 90%, 91%, 92%, 93%, 94%, 95%, 96%, 97%, 98%, 99%, 99.5% or more identical to the nucleotide sequence set forth in SEQ ID NO: 2;
(d) a nucleotide sequence that is at least about 70%, 75%, 80%, 85%, 90%, 91%, 92%, 93%, 94%, 95%, 96%, 97%, 98%, 99%, 99.5% or more identical to the nucleotide sequence set forth in SEQ ID NO: 3;
(e) a nucleotide sequence that is at least about 70%, 75%, 80%, 85%, 90%, 91%, 92%, 93%, 94%, 95%, 96%, 97%, 98%, 99%, 99.5% or more identical to the nucleotide sequence set forth in SEQ ID NO: 4;
(f) a nucleotide sequence that is at least about 70%, 75%, 80%, 85%, 90%, 91%, 92%, 93%, 94%, 95%, 96%, 97%, 98%, 99%, 99.5% or more identical to the nucleotide sequence set forth in SEQ ID NO: 5;
(g) a nucleotide sequence that is at least about 70%, 75%, 80%, 85%, 90%, 91%, 92%, 93%, 94%, 95%, 96%, 97%, 98%, 99%, 99.5% or more identical to the nucleotide sequence set forth in SEQ ID NO: 6;
(h) one or more nucleotide sequences that encodes a polypeptide comprising, consisting essentially of or consisting of the amino acid sequence set forth in any one of SEQ ID NOs: 7-10;
(i) a nucleotide sequence that encodes a polypeptide comprising, consisting essentially of or consisting of an amino acid sequence that is at least about 70%, 75%, 80%, 85%, 90%, 91%, 92%, 93%, 94%, 95%, 96%, 97%, 98%, 99%, 99.5% or more identical to the amino acid sequence set forth in SEQ ID NO: 7;
(j) a nucleotide sequence that encodes a polypeptide comprising, consisting essentially of or consisting of an amino acid sequence that is at least about 70%, 75%, 80%, 85%, 90%, 91%, 92%, 93%, 94%, 95%, 96%, 97%, 98%, 99%, 99.5% or more identical to the amino acid sequence set forth in SEQ ID NO: 8;
(k) a nucleotide sequence that encodes a polypeptide comprising, consisting essentially of or consisting of an amino acid sequence that is at least about 70%, 75%, 80%, 85%, 90%, 91%, 92%, 93%, 94%, 95%, 96%, 97%, 98%, 99%, 99.5% or more identical to the amino acid sequence set forth in SEQ ID NO: 9;
(l) a nucleotide sequence that encodes a polypeptide comprising, consisting essentially of or consisting of an amino acid sequence that is at least about 70%, 75%, 80%, 85%, 90%, 91%, 92%, 93%, 94%, 95%, 96%, 97%, 98%, 99%, 99.5% or more identical to the amino acid sequence set forth in SEQ ID NO: 10;
(m) a nucleotide sequence that is complementary to any one of the nucleotide sequences described in (a) to (l) above;
(n) a nucleotide sequence that hybridizes to any one of the nucleotide sequences described in (a) to (m) above under stringent hybridization conditions;
(o) a functional fragment of any one of the nucleotide sequences described in (a), (b), (d), (e), (g), (h), (i) and (k) above, wherein the functional fragment encodes a δ-endotoxin;
(p) a functional fragment of any one of the nucleotide sequences described in (a), (b), (d), (h) and (i) above, wherein the functional fragment encodes a polypeptide that comprises an N-terminal helical bundle domain that is at least about 70%, 75%, 80%, 85%, 90%, 91%, 92%, 93%, 94%, 95%, 96%, 97%, 98%, 99%, 99.5% or more identical to amino acids 72 to 286 of SEQ ID NO: 7, a central beta-sheet domain that is at least about 70%, 75%, 80%, 85%, 90%, 91%, 92%, 93%, 94%, 95%, 96%, 97%, 98%, 99%, 99.5% or more identical to amino acids 295 to 511 of SEQ ID NO: 7, and a C-terminal beta-sandwich domain that is at least about 70%, 75%, 80%, 85%, 90%, 91%, 92%, 93%, 94%, 95%, 96%, 97%, 98%, 99%, 99.5% or more identical to amino acids 514 to 675 of SEQ ID NO: 7;
(q) a functional fragment of any one of the nucleotide sequences described in (a), (e), (g), (h) and (k) above, wherein the functional fragment encodes a polypeptide that comprises an N-terminal helical bundle domain that is at least about 70%, 75%, 80%, 85%, 90%, 91%, 92%, 93%, 94%, 95%, 96%, 97%, 98%, 99%, 99.5% or more identical to amino acids 51 to 271 of SEQ ID NO: 9, a central beta-sheet domain that is at least about 70%, 75%, 80%, 85%, 90%, 91%, 92%, 93%, 94%, 95%, 96%, 97%, 98%, 99%, 99.5% or more identical to amino acids 279 to 481 of SEQ ID NO: 9, and a C-terminal beta-sandwich domain that is at least about 70%, 75%, 80%, 85%, 90%, 91%, 92%, 93%, 94%, 95%, 96%, 97%, 98%, 99%, 99.5% or more identical to amino acids 486 to 646 of SEQ ID NO: 9; and/or
(r) a functional fragment of any one of the nucleotide sequences described in (a), (c), (d), (f), (g), (h), (j) and (l) above, wherein the functional fragment encodes a protein the expression of which increases the expression, stability and/or activity of one or more δ-endotoxins; and
   producing a plant from the plant or plant part, wherein the plant so produced comprises the exogenous nucleic acid (or a functional fragment thereof) and exhibits enhanced pest resistance as compared to a control plant of the same species grown under the same environmental conditions.

In some embodiments, methods of producing plants having enhanced pest resistance comprise, consist essentially of or consist of crossing a first parent plant or plant part with a second parent plant or plant part, wherein the first parent plant or plant part comprises within its genome a nucleic acid (e.g., an exogenous nucleic acid) comprising, consisting essentially of or consisting of:
(a) one or more of the nucleotide sequences set forth in SEQ ID NOs: 1-6;
(b) a nucleotide sequence that is at least about 70%, 75%, 80%, 85%, 90%, 91%, 92%, 93%, 94%, 95%, 96%, 97%, 98%, 99%, 99.5% or more identical to the nucleotide sequence set forth in SEQ ID NO: 1;
(c) a nucleotide sequence that is at least about 70%, 75%, 80%, 85%, 90%, 91%, 92%, 93%, 94%, 95%, 96%, 97%, 98%, 99%, 99.5% or more identical to the nucleotide sequence set forth in SEQ ID NO: 2;
(d) a nucleotide sequence that is at least about 70%, 75%, 80%, 85%, 90%, 91%, 92%, 93%, 94%, 95%, 96%, 97%, 98%, 99%, 99.5% or more identical to the nucleotide sequence set forth in SEQ ID NO: 3;
(e) a nucleotide sequence that is at least about 70%, 75%, 80%, 85%, 90%, 91%, 92%, 93%, 94%, 95%, 96%, 97%, 98%, 99%, 99.5% or more identical to the nucleotide sequence set forth in SEQ ID NO: 4;
(f) a nucleotide sequence that is at least about 70%, 75%, 80%, 85%, 90%, 91%, 92%, 93%, 94%, 95%, 96%, 97%, 98%, 99%, 99.5% or more identical to the nucleotide sequence set forth in SEQ ID NO: 5;
(g) a nucleotide sequence that is at least about 70%, 75%, 80%, 85%, 90%, 91%, 92%, 93%, 94%, 95%, 96%, 97%, 98%, 99%, 99.5% or more identical to the nucleotide sequence set forth in SEQ ID NO: 6;
(h) one or more nucleotide sequences that encodes a polypeptide comprising, consisting essentially of or consisting of the amino acid sequence set forth in any one of SEQ ID NOs: 7-10;
(i) a nucleotide sequence that encodes a polypeptide comprising, consisting essentially of or consisting of an amino acid sequence that is at least about 70%, 75%, 80%, 85%, 90%, 91%, 92%, 93%, 94%, 95%, 96%, 97%, 98%, 99%, 99.5% or more identical to the amino acid sequence set forth in SEQ ID NO: 7;
(j) a nucleotide sequence that encodes a polypeptide comprising, consisting essentially of or consisting of an amino acid sequence that is at least about 70%, 75%, 80%, 85%, 90%, 91%, 92%, 93%, 94%, 95%, 96%, 97%, 98%, 99%, 99.5% or more identical to the amino acid sequence set forth in SEQ ID NO: 8;
(k) a nucleotide sequence that encodes a polypeptide comprising, consisting essentially of or consisting of an amino acid sequence that is at least about 70%, 75%, 80%, 85%, 90%, 91%, 92%, 93%, 94%, 95%, 96%, 97%, 98%, 99%, 99.5% or more identical to the amino acid sequence set forth in SEQ ID NO: 9;
(l) a nucleotide sequence that encodes a polypeptide comprising, consisting essentially of or consisting of an amino acid sequence that is at least about 70%, 75%, 80%, 85%, 90%, 91%, 92%, 93%, 94%, 95%, 96%, 97%, 98%, 99%, 99.5% or more identical to the amino acid sequence set forth in SEQ ID NO: 10;
(m) a nucleotide sequence that is complementary to any one of the nucleotide sequences described in (a) to (l) above;
(n) a nucleotide sequence that hybridizes to any one of the nucleotide sequences described in (a) to (m) above under stringent hybridization conditions;
(o) a functional fragment of any one of the nucleotide sequences described in (a), (b), (d), (e), (g), (h), (i) and (k) above, wherein the functional fragment encodes a δ-endotoxin;
(p) a functional fragment of any one of the nucleotide sequences described in (a), (b), (d), (h) and (i) above, wherein the functional fragment encodes a polypeptide that comprises an N-terminal helical bundle domain that is at least about 70%, 75%, 80%, 85%, 90%, 91%, 92%, 93%, 94%, 95%, 96%, 97%, 98%, 99%, 99.5% or more identical to amino acids 72 to 286 of SEQ ID NO: 7, a central beta-sheet domain that is at least about 70%, 75%, 80%, 85%, 90%, 91%, 92%, 93%, 94%, 95%, 96%, 97%, 98%, 99%, 99.5% or more identical to amino acids 295 to 511 of SEQ ID NO: 7, and a C-terminal beta-sandwich domain that is at least about 70%, 75%, 80%, 85%, 90%, 91%, 92%, 93%, 94%, 95%, 96%, 97%, 98%, 99%, 99.5% or more identical to amino acids 514 to 675 of SEQ ID NO: 7;
(q) a functional fragment of any one of the nucleotide sequences described in (a), (e), (g), (h) and (k) above, wherein the functional fragment encodes a polypeptide that comprises an N-terminal helical bundle domain that is at least about 70%, 75%, 80%, 85%, 90%, 91%, 92%, 93%, 94%, 95%, 96%, 97%, 98%, 99%, 99.5% or more identical to amino acids 51 to 271 of SEQ ID NO: 9, a central beta-sheet domain that is at least about 70%, 75%, 80%, 85%, 90%, 91%, 92%, 93%, 94%, 95%, 96%, 97%, 98%, 99%, 99.5% or more identical to amino acids 279 to 481 of SEQ ID NO: 9, and a C-terminal beta-sandwich domain that is at least about 70%, 75%, 80%, 85%, 90%, 91%, 92%, 93%, 94%, 95%, 96%, 97%, 98%, 99%, 99.5% or more identical to amino acids 486 to 646 of SEQ ID NO: 9; and/or
(r) a functional fragment of any one of the nucleotide sequences described in (a), (c), (d), (f), (g), (h), (j) and (l) above, wherein the functional fragment encodes a protein the expression of which increases the expression, stability and/or activity of one or more δ-endotoxins,
   thereby producing a progeny generation that comprises at least one plant that comprises the nucleic acid (or a functional fragment thereof) and that exhibits enhanced pest resistance as compared to a control plant of the same species grown under the same environmental conditions. Such methods may further comprise selecting a progeny plant or plant part that comprises the nucleic acid (or a functional fragment thereof) within its genome and that exhibits enhanced pest resistance as compared to a control plant of the same species grown under the same environmental conditions. Such selections may be made based upon the presence of the nucleic acid (or a functional fragment thereof) and/or the enhanced pest resistance of the progeny plant or part.

In some embodiments, methods of producing plants having enhanced pest resistance comprise, consist essentially of or consist of crossing a first plant or plant part that comprises a nucleic acid (e.g., an exogenous nucleic acid) encoding one or more CRY71 proteins, one or more CRY72 proteins and/or one or more ORF2 proteins with a second plant or plant part that lacks the nucleic acid and repeatedly backcrossing progeny plants comprising the nucleic acid (or a functional fragment thereof) with the second plant or plant part to produce an introgressed plant or plant part that comprises the nucleic acid (or a functional fragment thereof) and that exhibits enhanced pest resistance as compared to a control plant of the same species grown under the same environmental conditions. In some embodiments, the method further comprises selecting the introgressed plant or plant part based upon the presence of the nucleic acid (or a functional fragment thereof) and/or its enhanced pest resistance. In some embodiments, the method further comprises selecting the introgressed plant or plant part (for inclusion in a breeding program, for example).

In some embodiments, methods of producing plants and plant parts having enhanced pest resistance comprise, consist essentially of or consist of crossing a first plant or plant part that comprises a nucleic acid (e.g., an exogenous nucleic acid)with a second plant or plant part that lacks the nucleic acid and repeatedly backcrossing progeny plants comprising the nucleic acid (or a functional fragment thereof) with the second plant or plant part to produce an introgressed plant or plant part that comprises the nucleic acid (or a functional fragment thereof) and that exhibits enhanced pest resistance as compared to a control plant of the same species grown under the same environmental conditions, wherein the exogenous nucleic acid comprises, consists essentially of or consists of:
(a) one or more of the nucleotide sequences set forth in SEQ ID NOs: 1-6;
(b) a nucleotide sequence that is at least about 70%, 75%, 80%, 85%, 90%, 91%, 92%, 93%, 94%, 95%, 96%, 97%, 98%, 99%, 99.5% or more identical to the nucleotide sequence set forth in SEQ ID NO: 1;
(c) a nucleotide sequence that is at least about 70%, 75%, 80%, 85%, 90%, 91%, 92%, 93%, 94%, 95%, 96%, 97%, 98%, 99%, 99.5% or more identical to the nucleotide sequence set forth in SEQ ID NO: 2;
(d) a nucleotide sequence that is at least about 70%, 75%, 80%, 85%, 90%, 91%, 92%, 93%, 94%, 95%, 96%, 97%, 98%, 99%, 99.5% or more identical to the nucleotide sequence set forth in SEQ ID NO: 3;
(e) a nucleotide sequence that is at least about 70%, 75%, 80%, 85%, 90%, 91%, 92%, 93%, 94%, 95%, 96%, 97%, 98%, 99%, 99.5% or more identical to the nucleotide sequence set forth in SEQ ID NO: 4;
(f) a nucleotide sequence that is at least about 70%, 75%, 80%, 85%, 90%, 91%, 92%, 93%, 94%, 95%, 96%, 97%, 98%, 99%, 99.5% or more identical to the nucleotide sequence set forth in SEQ ID NO: 5;
(g) a nucleotide sequence that is at least about 70%, 75%, 80%, 85%, 90%, 91%, 92%, 93%, 94%, 95%, 96%, 97%, 98%, 99%, 99.5% or more identical to the nucleotide sequence set forth in SEQ ID NO: 6;
(h) one or more nucleotide sequences that encodes a polypeptide comprising, consisting essentially of or consisting of the amino acid sequence set forth in any one of SEQ ID NOs: 7-10;
(i) a nucleotide sequence that encodes a polypeptide comprising, consisting essentially of or consisting of an amino acid sequence that is at least about 70%, 75%, 80%, 85%, 90%, 91%, 92%, 93%, 94%, 95%, 96%, 97%, 98%, 99%, 99.5% or more identical to the amino acid sequence set forth in SEQ ID NO: 7;
(j) a nucleotide sequence that encodes a polypeptide comprising, consisting essentially of or consisting of an amino acid sequence that is at least about 70%, 75%, 80%, 85%, 90%, 91%, 92%, 93%, 94%, 95%, 96%, 97%, 98%, 99%, 99.5% or more identical to the amino acid sequence set forth in SEQ ID NO: 8;
(k) a nucleotide sequence that encodes a polypeptide comprising, consisting essentially of or consisting of an amino acid sequence that is at least about 70%, 75%, 80%, 85%, 90%, 91%, 92%, 93%, 94%, 95%, 96%, 97%, 98%, 99%, 99.5% or more identical to the amino acid sequence set forth in SEQ ID NO: 9;
(l) a nucleotide sequence that encodes a polypeptide comprising, consisting essentially of or consisting of an amino acid sequence that is at least about 70%, 75%, 80%, 85%, 90%, 91%, 92%, 93%, 94%, 95%, 96%, 97%, 98%, 99%, 99.5% or more identical to the amino acid sequence set forth in SEQ ID NO: 10;
(m) a nucleotide sequence that is complementary to any one of the nucleotide sequences described in (a) to (l) above;
(n) a nucleotide sequence that hybridizes to any one of the nucleotide sequences described in (a) to (m) above under stringent hybridization conditions;
(o) a functional fragment of any one of the nucleotide sequences described in (a), (b), (d), (e), (g), (h), (i) and (k) above, wherein the functional fragment encodes a δ-endotoxin;
(p) a functional fragment of any one of the nucleotide sequences described in (a), (b), (d), (h) and (i) above, wherein the functional fragment encodes a polypeptide that comprises an N-terminal helical bundle domain that is at least about 70%, 75%, 80%, 85%, 90%, 91%, 92%, 93%, 94%, 95%, 96%, 97%, 98%, 99%, 99.5% or more identical to amino acids 72 to 286 of SEQ ID NO: 7, a central beta-sheet domain that is at least about 70%, 75%, 80%, 85%, 90%, 91%, 92%, 93%, 94%, 95%, 96%, 97%, 98%, 99%, 99.5% or more identical to amino acids 295 to 511 of SEQ ID NO: 7, and a C-terminal beta-sandwich domain that is at least about 70%, 75%, 80%, 85%, 90%, 91%, 92%, 93%, 94%, 95%, 96%, 97%, 98%, 99%, 99.5% or more identical to amino acids 514 to 675 of SEQ ID NO: 7;
(q) a functional fragment of any one of the nucleotide sequences described in (a), (e), (g), (h) and (k) above, wherein the functional fragment encodes a polypeptide that comprises an N-terminal helical bundle domain that is at least about 70%, 75%, 80%, 85%, 90%, 91%, 92%, 93%, 94%, 95%, 96%, 97%, 98%, 99%, 99.5% or more identical to amino acids 51 to 271 of SEQ ID NO: 9, a central beta-sheet domain that is at least about 70%, 75%, 80%, 85%, 90%, 91%, 92%, 93%, 94%, 95%, 96%, 97%, 98%, 99%, 99.5% or more identical to amino acids 279 to 481 of SEQ ID NO: 9, and a C-terminal beta-sandwich domain that is at least about 70%, 75%, 80%, 85%, 90%, 91%, 92%, 93%, 94%, 95%, 96%, 97%, 98%, 99%, 99.5% or more identical to amino acids 486 to 646 of SEQ ID NO: 9; and/or
(r) a functional fragment of any one of the nucleotide sequences described in (a), (c), (d), (f), (g), (h), (j) and (l) above, wherein the functional fragment encodes a protein the expression of which increases the expression, stability and/or activity of one or more δ-endotoxins.
   In some embodiments, the method further comprises selecting the introgressed plant or plant part based upon the presence of the nucleic acid (or a functional fragment thereof) and/or its enhanced pest resistance. In some embodiments, the method further comprises selecting the introgressed plant or plant part (for inclusion in a breeding program, for example).

Any suitable nucleic acid may be detected in/introduced into the plant or plant part, including, but not limited to, nucleic acids of the present invention. In some embodiments, the nucleic acid detected in/introduced into the plant or plant part is a nucleic acid encoding one or more of SEQ ID NOs: 7-10 (e.g., an exogenous nucleic acid comprising one or more of SEQ ID NOs: 1-6).

Exogenous nucleic acids may be introduced into a plant or plant part via any suitable method, including, but not limited to, microparticle bombardment, liposome-mediated transfection, receptor-mediated delivery, bacteria-mediated delivery (e.g., Agrobacterium-mediated transformation and/or whiskers-mediated transformation). In some embodiments, the exogenous nucleic acid is introduced into a plant part by crossing a first plant or plant part comprising the exogenous nucleic acid with a second plant or plant part that lacks the exogenous nucleic acid.

Nucleic acids encoding CRY71 proteins and/or CRY72 proteins may be detected using any suitable method, including, but not limited to, DNA sequencing, mass spectrometry and capillary electrophoresis. In some embodiments, the nucleic acid (or an informative fragment thereof) is detected in one or more amplification products from a nucleic acid sample from the plant or plant part. In some such embodiments, the amplification product(s) comprise(s) the nucleotide sequence of any one of SEQ ID NOs: 1-6, the reverse complement thereof, an informative fragment thereof, or an informative fragment of the reverse complement thereof.

Nucleic acids encoding CRY71 proteins and/or CRY72 proteins may be detected using any suitable probe. In some embodiments, the nucleic acid (or an informative fragment thereof) is detected using a probe comprising the nucleotide sequence of any one of SEQ ID NOs: 1-6, the reverse complement thereof, an informative fragment thereof, or an informative fragment of the reverse complement thereof In some embodiments, the probe comprises one or more detectable moieties, such as digoxigenin, fluorescein, acridine-ester, biotin, alkaline phosphatase, horseradish peroxidase, β-glucuronidase, β-galactosidase, luciferase, ferritin or a radioactive isotope.

Methods of the present invention may be used to identify, select and/or produce plants and/or plant parts that exhibit a variety of pest resistant phenotypes, including, but not limited to, increased survival rate, increased growth rate, increased height, increased chlorophyll content and/or increased yield (e.g., increased biomass, increased seed yield, increased YGSMN, increased GWTPN, increased PYREC, and/or decreased YRED) when grown under pest stress conditions (e.g., Acarina-, Anoplura-, Coleoptera-, Dermaptera-, Diptera-, Hemiptera-, Heteroptera-, Homoptera-, Hymenoptera-, Isoptera-, Lepidoptera-, Mallophaga-, Orthoptera-, Psocoptera-, Siphoptera-, Thysanoptera-, Thysanura-, and/or Trichoptera-stress conditions). In some embodiments, one or more pest resistant phenotypes is increased by at least about 5%, 10%, 15%, 20%, 25%, 30%, 35%, 40%, 45%, 50%, 55%, 60%, 65%, 75%, 80%, 85%, 90%, 95%, 100%, 125%, 150%, 175%, 200%, 250%, 300%, or more as compared to a control plant or plant part (e.g., a native plant of the same species) when each is grown under the same (or substantially the same) environmental conditions.

In some embodiments, the yield (e.g., seed yield, biomass, GWTPN, PYREC and/or YGSMN) of the plant or plant part is increased by at least about 5%, 10%, 15%, 20%, 25%, 30%, 35%, 40%, 45%, 50%, 55%, 60%, 65%, 75%, 80%, 85%, 90%, 95%, 100%, 125%, 150%, 175%, 200%, 250%, 300% or more as compared to a control plant or plant part (e.g., a native plant of the same species) grown under the same (or substantially the same) environmental conditions. For example, the seed yield and/or biomass of the plant or plant part may be increased by at least about 5%, 10%, 15%, 20%, 25%, 30%, 35%, 40%, 45%, 50%, 55%, 60%, 65%, 75%, 80%, 85%, 90%, 95%, 100%, 125%, 150%, 175%, 200%, 250%, 300% or more as compared to a control plant or plant part grown under the same (or substantially the same) Acarina- and/or insect-stress conditions.

Methods of the present invention may be used to identify, select, produce and/or protect plants and/or plant parts of any suitable plant type, including, but not limited to, plants belonging to the superfamily Viridiplantae. In some embodiments the plant or plant part is a fodder crop, a food crop, an ornamental plant, a tree or a shrub. For example, in some embodiments, the plant or plant part is a variety of Acer spp., Actinidia spp., Abelmoschus spp., Agropyron spp., Allium spp., Amaranthus spp., Ananas comosus, Annona spp., Apium graveolens, Arachis spp, Artocarpus spp., Asparagus officinalis, Avena spp. (e.g. Avena sativa, Avena fatua, Avena byzantina, Avena fatua var. sativa, Avena hybrida), Averrhoa carambola, Benincasa hispida, Bertholletia excelsea, Beta vulgaris, Brassica spp. (e.g. Brassica napus, Brassica rapa ssp. [canola, oilseed rape, turnip rape]), Cadaba farinosa, Camellia sinensis, Canna indica, Capsicum spp., Carex elata, Carica papaya, Carissa macrocarpa, Carya spp., Carthamus tinctorius, Castanea spp., Cichorium endivia, Cinnamomum spp., Citrullus lanatus, Citrus spp., Cocos spp., Coffea spp., Colocasia esculenta, Cola spp., Coriandrum sativum, Corylus spp., Crataegus spp., Crocus sativus, Cucurbita spp., Cucumis spp., Cynara spp., Daucus carota, Desmodium spp., Dimocarpus longan, Dioscorea spp., Diospyros spp., Echinochloa spp., Elaeis (e.g. Elaeis guineensis, Elaeis oleifera), Eleusine coracana, Eriobotrya japonica, Eugenia uniflora, Fagopyrum spp., Fagus spp., Ficus carica, Fortunella spp., Fragaria spp., Ginkgo biloba, Glycine spp. (e.g. Glycine max, Soja hispida or Soja max), Gossypium hirsutum, Helianthus spp. (e.g. Helianthus annuus), Hemerocallis fulva, Hibiscus spp., Hordeum spp. (e.g. Hordeum vulgare), Ipomoea batatas, Juglans spp., Lactuca sativa, Lathyrus spp., Lens culinaris, Linum usitatissimum, Litchi chinensis, Lotus spp., Luffa acutangula, Lupinus spp., Luzula sylvatica, Lycopersicon spp. (e.g. Lycopersicon esculentum, Lycopersicon lycopersicum, Lycopersicon pyriforme), Macrotyloma spp., Malus spp., Malpighia emarginata, Mammea americana, Mangifera indica, Manihot spp., Manilkara zapota, Medicago sativa, Melilotus spp., Mentha spp., Miscanthus spp., Momordica spp., Morus nigra, Musa spp., Nicotiana spp., Olea spp., Opuntia spp., Ornithopus spp., Oryza spp. (e.g. Oryza sativa, Oryza latifolia), Panicum miliaceum, Passiflora edulis, Pastinaca sativa, Persea spp., Petroselinum crispum, Phaseolus spp., Phoenix spp., Physalis spp., Pinus spp., Pistacia vera, Pisum spp., Poa spp., Populus spp., Prosopis spp., Prunus spp., Psidium spp., Punica granatum, Pyrus communis, Quercus spp., Raphanus sativus, Rheum rhabarbarum, Ribes spp., Ricinus communis, Rubus spp., Saccharum spp., Sambucus spp., Secale cereale, Sesamum spp., Sinapis sp., Solanum spp. (e.g. Solanum tuberosum, Solanum integrifolium or Solanum lycopersicum), Sorghum bicolor, Spinacia spp., Syzygium spp., Tagetes spp., Tamarindus indica, Theobroma cacao, Trifolium spp., Triticosecale rimpaui, Triticum spp. (e.g. Triticum aestivum, Triticum durum, Triticum turgidum, Triticum hybernum, Triticum macha, Triticum sativum or Triticum vulgare), Tropaeolum minus, Tropaeolum majus, Vaccinium spp., Vicia spp., Vigna spp., Viola odorata, Vitis spp., Zea mays, Zizania palustris or Ziziphus spp., amongst others.

In some embodiments, the plant or plant part is a rice, maize, wheat, barley, sorghum, millet, oat, triticale, rye, buckwheat, fonio, quinoa, sugar cane, bamboo, banana, ginger, onion, lily, daffodil, iris, amaryllis, orchid, canna, bluebell, tulip, garlic, secale, einkorn, spelt, emmer, durum, kamut, grass (e.g., gramma grass), teff, milo, flax, Tripsacum sp., or teosinte plant or plant part. In some embodiments, the plant or plant part is a blackberry, raspberry, strawberry, barberry, bearberry, blueberry, coffee berry, cranberry, crowberry, currant, elderberry, gooseberry, goji berry, honeyberry, lemon, lime, lingonberry, mangosteen, orange, pepper, persimmon, pomegranate, prune, cotton, clover, acai, plum, peach, nectarin, cherry, guava, almond, pecan, walnut, apple, amaranth, sweet pea, pear, potato, soybean, sugar beet, sunflower, sweet potato, tamarind, tea, tobacco or tomato plant or plant part.

The present invention extends to products harvested from plants and plant parts produced according to methods of the present invention, including, but not limited to, plant cells and harvestable plant parts such as seeds, leaves, fruits, flowers, stems, rhizomes, tubers and bulbs. In some embodiments, the harvested product is a plant part capable of producing a plant or plant part that expresses one or more CRY71 proteins, and/or one or more CRY72 proteins, and/or that exhibits enhanced pest resistance (e.g., enhanced Acarina and/or insect resistance). In some embodiments, the harvested product is a plant part capable of producing a plant or plant that exhibits increased survival rate, increased growth rate, increased height, increased chlorophyll content and/or increased yield (e.g., increased biomass, increased seed yield, increased YGSMN, increased GWTPN, increased PYREC, and/or decreased YRED) when grown under pest stress conditions (e.g., e.g., Acarina-, Anoplura-, Coleoptera-, Dermaptera-, Diptera-, Hemiptera-, Heteroptera-, Homoptera-, Hymenoptera-, Isoptera-, Lepidoptera-, Mallophaga-, Orthoptera-, Psocoptera-, Siphoptera-, Thysanoptera-, Thysanura-, and/or Trichoptera-stress conditions).

The present invention also extends to products derived from plants produced according to methods of the present invention, including, but not limited to, dry pellets and powders, oils, fats, fatty acids, starches and proteins.

The present invention also encompasses methods of controlling pests and of protecting plants and plant parts. Such methods may comprise, consist essentially of or consist of: expressing a nucleic acid/protein of the present invention in a plant or plant part; applying a pesticidal composition of the present invention to a plant or plant part in a pesticidally effective amount; and/or applying a pesticidal composition of the present invention to the pest and/or the pest's environment in a pesticidally effective amount.

Methods of the present invention may be used to control any suitable pest(s), including, but not limited to, pests belonging to the order Acarina, pests belonging to the order Anoplura, pests belonging to the order Coleoptera, pests belonging to the order Dermaptera, pests belonging to the order Diptera, pests belonging to the order Hemiptera, pests belonging to the order Heteroptera, pests belonging to the order Homoptera, pests belonging to the order Hymenoptera, pests belonging to the order Isoptera, pests belonging to the order Lepidoptera, pests belonging to the order Mallophaga, pests belonging to the order Orthoptera, pests belonging to the order Psocoptera, pests belonging to the order Siphoptera, pests belonging to the order Thysanoptera, pests belonging to the order Thysanura, and pests belonging to the order Trichoptera.

The Cry proteins of the invention can be used in combination with other Bacillus thuringiensis Cry proteins or other pesticidal principles to increase pest target range or for the prevention and/or management of insect resistance. Other pesticidal principles include without limitation insecticidal, fungicidal or nematicidal principles. Such insecticidal principles include biological insecticidal principles such as vegetative insecticidal proteins, such as Vip1, Vip2 and Vip3, protease inhibitors (both serine and cysteine types), lectins, alpha-amylase, peroxidase and cholesterol oxidase. Such chemical insecticidal principles include, without limitation, dinotefuran, thiamethoxam, imidacloprid, acetamiprid, nitenpyram, nidinotefuran, chlorfenapyr, tebufenpyrad, tebufenozide, methoxyfenozide, halofenozide, triazamate, avermectin, spinosad, fiprinol, acephate, fenamiphos, diazinon, chlorpyrifos, chlorpyrifon-methyl, malathion, carbaryl, aldicarb, carbofuran, thiodicarb, and oxamyl.

### EXAMPLES

The following examples are not intended to be a detailed catalog of all the different ways in which the present invention may be implemented or of all the features that may be added to the present invention. Persons skilled in the art will appreciate that numerous variations and additions to the various embodiments may be made without departing from the present invention. Hence, the following descriptions are intended to illustrate some particular embodiments of the invention, and not to exhaustively specify all permutations, combinations and variations thereof

### Example 1

### Isolation of Bt cry71Aa1 Gene and Recombinant Protein Expression

Bacillus thuringiensis strain HS18-1, obtained and isolated from the soil of primeval forest regions in Muchuan (Sichuan province, China), was found to exhibit extreme toxicity toward Lepidoptera pests, Diptera pests, etc. This strain was deposited under Accession No. 2718 at the China General Microbiological Culture Collection Center (CGMCC, 3a Datun Road, Chaoyang District, Beijing, Institute Of Microbiology Chinese Academy of Sciences, 100101), and disclosed in application number ZL200910081594.2.

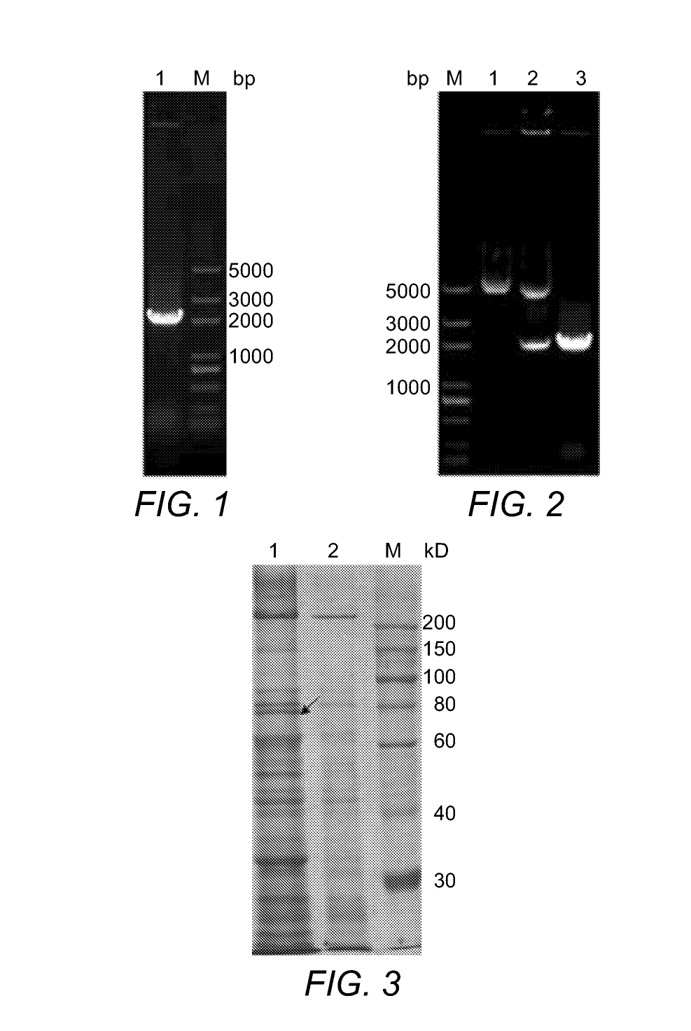

A DNA purification kit (SBS Genetech Co., Ltd.) was used to extract the total DNA from strain HS18-1. Total DNA was used as a template for amplifying the cry71Aa1 gene using forward primer pS71-F: GCC GGA TCC AAT GAA TTC ATA TCA AAG TGA A (SEQ ID NO: 11) and reverse primer pS71-R: GGG GTC GAC CTA CTT TGT TTT AAA TAA ACT (SEQ ID NO: 12), wherein the BamHI (GGATCC) and SalI (GTCGAC) enzyme digestion sites are underlined. The 25 μl PCR amplification reaction included 2.5 μl 10× buffer, 1.5 μl MgCl₂ (25 mM), 0.2 μl Taq DNA polymerase, 2 μl dNTPs (2.5 mM), 1 μl pS71-F primer, 1 μl pS71-R primer, 5 μl template DNA and 11.8 μl double distilled water. The thermocycling reaction included pre-denaturation at 94° C. for 5 minutes; 30 cycles of: denaturation at 94° C. for 50 seconds, annealing at 54° C. for 50 seconds, and extension at 72° C. for 2 minutes; and a final extension at 72° C. for 10 minutes. The amplification reaction products were subjected to electrophoresis in 0.7% agarose gel, and placed into a gel imaging system for observing the PCR amplification products (FIG. 1). A 2151 bp amplicon encoding Cry71Aa1 protein was obtained. The nucleotide sequence of the amplicon was analyzed and shown to have a GC content of 35.15%. The nucleotide sequence of the cry71Aa1 gene is provided herein as SEQ ID NO: 1.

Using the bacterial sigma70 promoter recognition program, a sequence having an RNA polymerase active site was identified upstream of the coding region. Further analysis indicated that the cry71Aa1 gene encoded a Cry71Aa1 protein of 716 amino acid residues, the sequence of which is provided herein as SEQ ID NO: 7. The amino acid composition of the Cry71Aa1 protein is provided in Table 1.

TABLE 1

| Amino Acid | Number | Percentage | Amino Acid | Number | Percentage |
|---|---|---|---|---|---|
| Ala(A): | 43 | 4.06 | Met(M): | 8 | 1.26 |
| Cys(C): | 10 | 1.28 | Asn(N): | 54 | 7.56 |
| Asp(D): | 34 | 4.80 | Pro(P): | 33 | 4.03 |
| Glu(E): | 38 | 5.93 | Gln(Q): | 29 | 4.49 |
| Phe(F): | 31 | 5.43 | Arg(R): | 36 | 6.65 |
| Gly(G): | 41 | 3.26 | Ser(S): | 63 | 7.02 |
| His(H): | 14 | 1.96 | Thr(T): | 56 | 7.07 |
| Ile(I): | 49 | 6.81 | Val(V): | 28 | 3.48 |
| Lys(K): | 32 | 4.96 | Trp(W): | 15 | 3.25 |
| Leu(L): | 61 | 8.48 | Tyr(Y): | 41 | 7.87 |

The PCR-amplified cry71Aa1 gene was digested with BamHI and SalI, and ligated into the BamHI and Sall sites of the shuttle vector pSTK. The recombinant plasmid was transformed into E. coli DH5α competent cells, the cells were grown, the plasmid was extracted, and the size of the insert was confirmed by electrophoresis (FIG. 2). The resulting recombinant plasmid was referred to as pSTK-cry71Aa1. To demethylate the plasmid DNA, the plasmid was transformed into E. coli Trans110 (Beijing TransGen Biotech Co., Ltd.). Subsequently, the plasmid was extracted and transferred into the Bacillus thuringiensis no-crystal mutant strain HD73⁻ by electroporation using the parameters 2.2 kV, 1000Ω and 25 μF. The recombinant strain containing the recombinant plasmid was referred to as HD71Aa1. As a negative control, the pSTK plasmid was also transformed into Bacillus thuringiensis no-crystal mutant strain HD73⁻. All transformants were cultivated in ½ Luria-Bertani (LB) medium at 28° C., 200 r/min for 72 hours. The culture solution was subsequently centrifuged to collect thalli, and the supernatant was discarded. The thalli were washed with sterile water three times; 30 mL 10 mmol/L Tris-HCl (pH 8.0) was added and the cells were disrupted by ultrasonication. Proteins were extracted and detected by SDS-PAGE. As shown in FIG. 3, SDS-PAGE analysis indicated that the HD71Aa1 transformant expressed an ˜80 kDa Cry71Aa1 protein, the molecular weight of which was consistent with the molecular weight of the predicted protein.

To microscopically observe the expression of the Cry71Aa1 protein in HD73⁻ cells, the transformants were cultivated in ½ LB medium at 28° C., 200 r/min, and thalli were collected after more than 90% sporulation. For optical microscopy, a slide was prepared for observing whether the transformants produced a crystal protein. Crystal morphology was also observed using a scanning electron microscope (Hitachi Co., Ltd., Japan). This analysis indicated that the Cry71Aa1 protein was expressed in HD73⁻, but parasporal crystals did not form.

To demonstrate insecticidal activity of the Cry71Aa1 protein against Spodoptera exigua, mixtures of spores and crystals were obtained after cultivating the HD71aA1 strain at 28° C., 200 r/min for 72 hours. The mixtures were prepared at five different concentrations: 13.7, 24, 48, 80.3, and 144.5 μg/mL. Subsequently, 45 1-year-old larvae of Spodoptera exigua were contacted with each mixture. Experiments were repeated three times with the pSTK plasmid transformed Bacillus thuringiensis no-crystal mutant strain HD73⁻ as a negative control, and clear water as a blank control. The results were counted after 72 hours, and LC₅₀ (medium lethal concentration, i.e., the amount of agent, which kills 50% of target organism) was analyzed using SPSS10.0 software. The results of this analysis are presented in Table 2.

TABLE 2

| | | 95% Confidence |
|---|---|---|
| Transformant | LC₅₀/(μg/mL) | Limit/(μg/mL) |
| HD71Aa1 | 210.1 | 76.1-193.9 |
| Negative Control | N | N |

N represents no insecticidal activity.

The results of this analysis indicated that the HD71Aa1 transformant exhibited insecticidal activity against the larvae of Spodoptera exigua with and LC₅₀ of 210.1 μg/mL. By comparison, the negative controls had no insecticidal activity against Spodoptera exigua.

### Example 2

### Isolation of Bt cry71Aa1 Operon and Recombinant Protein Expression

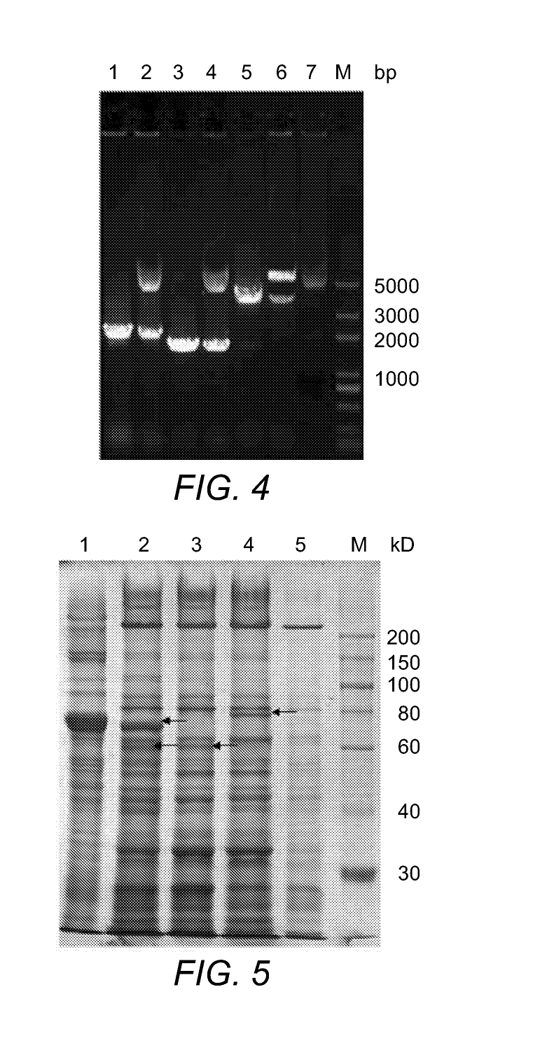

Total DNA from strain HS18-1 was extracted, as described in Example 1, and used as a template for PCR amplification of the cry71Aa1 operon. The PCR reaction included 1.5 μl forward primer 71AF: 5′-ATG AAT TCA TAT CAA AGT GAA-3′ (SEQ ID NO: 15), 1.5 μl reverse primer 71AR: 5′-TTA ACG TTT ATA TCC TTG ACT A-3′ (SEQ ID NO: 16), 2 μl template DNA, 7.5 μl double distilled water and 12.5 μl TAQMIX (Biomed Biotech, Beijing, China). The thermocycling reaction included pre-denaturation at 94° C. for 5 minutes; 30 cycles of: denaturation at 94° C. for 50 seconds, annealing at 54° C. for 50 seconds, and extension at 72° C. for 3.5 minutes; and a final extension at 72° C. for 10 minutes. The amplification reaction products were subjected to electrophoresis in 0.7% agarose gel, and placed into a gel imaging system for observing the PCR amplification products (FIG. 4). A 3819 bp amplicon containing the cry71Aa1 operon was obtained. Nucleotide sequence analysis of the operon indicated the presence of the 2151 bp cry71Aa1 gene (SEQ ID NO: 1), a 1611 bp Cry71orf2 gene (SEQ ID NO: 2), and a 57 bp non-coding spacer therebetween. The full-length operon is provided herein as SEQ ID NO: 3.

Sequence analysis indicated that the Cry71orf2 gene encoded a protein of 536 amino acid residues, the sequence of which is provided herein as SEQ ID NO: 8. The amino acid composition of the Cry71Orf2 protein is provided in Table 3.

TABLE 3

| Amino Acid | Number | Percentage | Amino Acid | Number | Percentage |
|---|---|---|---|---|---|
| Ala(A): | 24 | 3 | Met(M): | 18 | 3.77 |
| Cys(C): | 10 | 1.7 | Asn(N): | 39 | 7.24 |
| Asp(D): | 37 | 6.92 | Pro(P): | 20 | 3.23 |
| Glu(E): | 35 | 7.23 | Gln(Q): | 31 | 6.36 |
| Phe(F): | 14 | 3.25 | Arg(R): | 18 | 4.4 |
| Gly(G): | 36 | 6.72 | Ser(S): | 32 | 4.72 |
| His(H): | 21 | 4.58 | Thr(T): | 38 | 6.36 |
| Ile(I): | 24 | 4.42 | Val(V): | 29 | 4.77 |
| Lys(K): | 32 | 6.57 | Trp(W): | 7 | 2.01 |
| Leu(L): | 34 | 6.26 | Tyr(Y): | 37 | 9.41 |

The open reading frames of the cry71Aa1 operon were amplified using gene-specific primers. Cry71Aa1 was amplified with pS71-F (SEQ ID NO: 11) and pS71-R (SEQ ID NO: 12), Cry71orf2 was amplified with pSO-F (5′-GCC GGA TCC AAT GTA TAC CAA TAC TAT GAA A-3′; SEQ ID NO: 13) and pSO-R (5′-GGG GTC GAC TTA ACG TTT ATA TCC TTG ACT A-3′; SEQ ID NO: 14), and the complete cry71Aa1 operon was amplified with pS71-F and pSO-R. Amplification was performed using total DNA from strain HS18-1 as a template. The resulting amplicons were digested with BamHI and SalI and inserted into vector pSTK. Recombinant plasmids were transformed and amplified in trans1-T1 competent cells (TransGen Biotech, Inc., China). Plasmids were extracted and subjected to enzyme digestion and electrophoresis to confirm the size of the insert. Plasmids were subsequently demethylated with trans110 competent cells (TransGen Biotech, Inc., China) and transferred into the no-crystal mutant strain HD73⁻ by electroporation (2.2 kV, 1000Ω, 25 μF). The recombinant plasmids were named pSTK-cry71Aa1, pSTK-Cry71orf2, and pSTK-cry71Aa1-Cry71orf2, and the transformants containing the recombinant plasmids were named HD71, HDO, and HD71O, respectively.

All transformants were cultivated in ½ LB medium at 30° C., 200 r/min for 72 hours. The culture solution was subsequently centrifuged to collect thalli, and the supernatant was discarded. The thalli were washed with sterile water three times; 30 mL 10 mmol/L Tris-HCl (pH 8.0) was added and the cells were disrupted by ultrasonication. Proteins were extracted and detected by SDS-PAGE. As shown in FIG. 5, SDS-PAGE analysis indicated that the HD71O transformant expressed two proteins having different molecular weights, one at about 80 kDa (Cry71Aa1 protein) and the other at about 60 kDa (Cry71Orf 2 protein), whereas transformant HD71 only expressed the cry71Aa1 protein and transformant HDO only expressed the Cry71Orf2 protein.

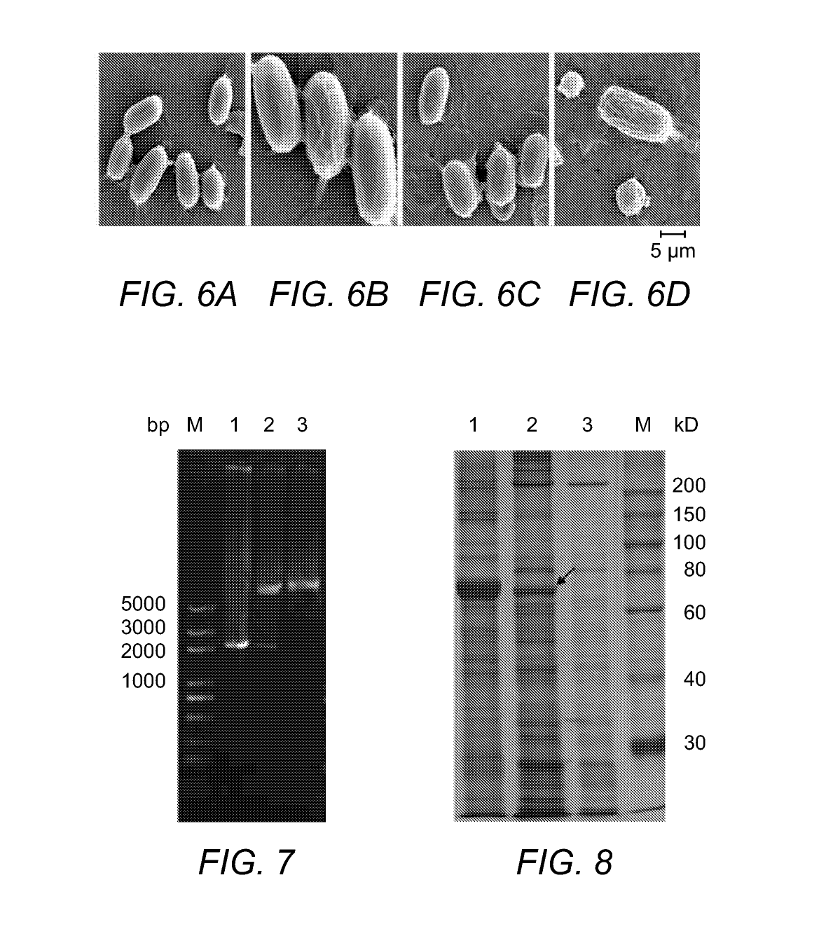

To microscopically observe the expression of the Cry71Aa1 and Cry71Orf2 proteins in HD73⁻ cells, the transformants were cultivated in ½ LB medium at 28° C., 200 r/min, and thalli were collected after more than 90% sporulation. For optical microscopy, a slide was prepared for observing whether the transformants produced a crystal protein. Crystal morphology was also observed using a scanning electron microscope (Hitachi Co., Ltd., Japan). This analysis indicated that the complete Cry71Aa1 operon gene was expressed in HD73⁻ and formed spherical parasporal crystals (FIG. 6D), whereas the cry71Aa1 gene alone was expressed in HD73⁻ at low levels (FIG. 6B), and the Cry71orf2 gene alone did not express the parasporal crystals (FIG. 6C).

To demonstrate insecticidal activity against Spodoptera exigua, mixtures of spores and crystals were obtained after cultivating the HD71, HDO, and HD71O strains at 28° C., 200 r/min for 72 hours. The mixtures were prepared at five different concentrations with HD71 at 13.7, 24, 48, 80.3, and 144.5 μg/mL; HDO at 10.5, 18.9, 34.0, 61.1, and 110.1 μg/mL; and HD71O at 1.34, 2.4, 4.3, 7.8, 14.8 and μg/mL. Subsequently, 45 1-year-old larvae of Spodoptera exigua were contacted with each mixture. Experiments were repeated three times with the pSTK plasmid transformed Bacillus thuringiensis no-crystal mutant strain HD73⁻ as a negative control, and clear water as a blank control. The results were counted after 12 hours, and LC₅₀ was analyzed using SPSS13.0 software. The results of this analysis are presented in Table 4.

TABLE 4

| | | 95% Confidence |
|---|---|---|
| Transformant | LC₅₀/(μg/mL) | Limit/(μg/mL) |
| HD71O | 28.61 | 15.51-133.84 |
| HD71 | 210.1 | 11.2-15.3 |
| HDO | N | N |
| Negative Control | N | N |

N represents no insecticidal activity.

The results of this analysis indicated that the expression product of the transformant HD71O exhibited good insecticidal activity against Spodoptera exigua with an LC₅₀ of 28.61 μg/mL. By comparison, the expression product of the transformant HD71 exhibited some insecticidal activity against Spodoptera exigua (LC₅₀ of 210.1 μg/mL), whereas neither the HDO transformant nor the negative control exhibited any insecticidal activity against Spodoptera exigua.

### Example 3

### Isolation of Bt cry72Aa1 Gene and Recombinant Protein Expression

Total DNA from strain HS18-1 was extracted, as described in Example 1, and used as a template for PCR amplification of the cry72Aa1 gene. Total DNA was used as a template for amplifying the cry72Aa1 gene using forward primer pS72-F: 5′-GGG GTC GAC AAT GTC TAA TCG TTA TCC ACG-3′ (SEQ ID NO: 17) and reverse primer pS72-R: 5′-CCC CTC GAG TTA TTT GAC AAA TAA ACT ATT-3′ (SEQ ID NO: 18), wherein the SalI (GTCGAC) and XhoI (CTCGAG) enzyme digestion sites are underlined. The 25 μl PCR amplification reaction included 2.5 μl 10× buffer, 1.5 μl MgCl₂ (25 mM), 0.2 Taq DNA polymerase, 2 μl dNTPs (2.5 mM), 1 μl pS72-F primer, 1 μl pS72-R primer, 5 μl template DNA and 11.8 μl double distilled water. The thermocycling reaction included pre-denaturation at 94° C. for 5 minutes; 30 cycles of: denaturation at 94° C. for 50 seconds, annealing at 54° C. for 50 seconds, and extension at 72° C. for 2 minutes; and a final extension at 72° C. for 10 minutes. The amplification reaction products were subjected to electrophoresis in 0.7% agarose gel, and placed into a gel imaging system for observing the PCR amplification products (FIG. 7). A 2064 bp amplicon encoding Cry72Aa1 protein was obtained. The nucleotide sequence of the amplicon was analyzed and shown to have a GC content of 36.87%. The nucleotide sequence of the cry72Aa1 gene is provided herein as SEQ ID NO:4.

Using the bacterial sigma70 promoter recognition program, a sequence having an RNA polymerase active site was identified upstream of the cry7 2Aa1 coding region. Further analysis indicated that the cry72Aa1 gene encoded a Cry72Aa1 protein of 687 amino acid residues, the sequence of which is provided herein as SEQ ID NO: 9. The amino acid composition of the Cry72Aa1 protein is provided in Table 5.

TABLE 5

| Amino Acid | Number | Percentage | Amino Acid | Number | Percentage |
|---|---|---|---|---|---|
| Ala(A): | 39 | 3.88 | Met(M): | 7 | 1.17 |
| Cys(C): | 10 | 1.35 | Asn(N): | 53 | 7.71 |
| Asp(D): | 37 | 5.5 | Pro(P): | 28 | 4.08 |
| Glu(E): | 34 | 4.95 | Gln(Q): | 24 | 3.49 |
| Phe(F): | 30 | 5.53 | Arg(R): | 39 | 7.58 |
| Gly(G): | 49 | 4.11 | Ser(S): | 66 | 7.74 |
| His(H): | 13 | 2.25 | Thr(T): | 43 | 5.72 |
| Ile(I): | 47 | 6.88 | Val(V): | 35 | 4.57 |
| Lys(K): | 25 | 4.08 | Trp(W): | 10 | 2.28 |
| Leu(L): | 60 | 8.78 | Tyr(Y): | 38 | 7.68 |

The PCR-amplified cry72Aa1 gene was digested with SalI and XhoI, and ligated into the SalI and XhoI sites of the shuttle vector pSTK. The recombinant plasmid was transformed into E. coli DH5α competent cells, the cells were grown, the plasmid was extracted, and the size of the insert was confirmed by electrophoresis (FIG. 7). The resulting recombinant plasmid was referred to as pSTK-cry72Aa1. To demethylate the plasmid DNA, the plasmid was transformed into E. coli Trans110 (Beijing TransGen Biotech Co., Ltd.). Subsequently, the plasmid was extracted and transferred into the Bacillus thuringiensis no-crystal mutant strain HD73⁻ by electroporation using the parameters 2.2 kV, 1000Ω and 25 μF. The recombinant strain containing the recombinant plasmid was referred to as HD72Aa1. As a negative control, the pSTK plasmid was also transformed into Bacillus thuringiensis no-crystal mutant strain HD73⁻. All transformants were cultivated in ½ LB medium at 28° C., 200 r/min for 72 hours. The culture solution was subsequently centrifuged to collect thalli, and the supernatant was discarded. The thalli were washed with sterile water three times; 30 mL 10 mmol/L Tris-HCl (pH 8.0) was added and the cells were disrupted by ultrasonication. Proteins were extracted and detected by SDS-PAGE. As shown in FIG. 8, SDS-PAGE analysis indicated that the HD72Aa1 transformant expressed an ˜77 kDa Cry72Aa1 protein, the molecular weight of which was consistent with the molecular weight of the predicted protein.

To microscopically observe the expression of the Cry72Aa1 protein in HD73⁻ cells, the transformants were cultivated in ½ LB medium at 28° C., 200 r/min, and thalli were collected after more than 90% sporulation. For optical microscopy, a slide was prepared for observing whether the transformants produced a crystal protein. Crystal morphology was also observed using a scanning electron microscope (Hitachi Co., Ltd., Japan). This analysis indicated that the Cry72Aa1 protein was expressed in HD73⁻, but parasporal crystals did not form.

To demonstrate insecticidal activity of the Cry72Aa1 protein, mixtures of spores and crystals were obtained after cultivating the HD72aA1 strain at 28° C., 200 r/min for 72 hours. The mixtures were prepared at five different concentrations: 1.5, 2.7, 4.8, 8.7, and 15.8 μg/mL. Subsequently, 45 1-year-old larvae of Spodoptera exigua, Plutella xylostella and Helicoverpa armigera were independently contacted with each mixture. Experiments were repeated three times, wherein the pSTK plasmid transformed Bacillus thuringiensis no-crystal mutant strain HD73⁻ served as a negative control, and clear water as a blank control. The results were counted after 72 hours, and LC₅₀ was analyzed using SPSS10.0 software. The results of this analysis are presented in Table 6.

TABLE 6

| | | 95% Confidence |
|---|---|---|
| Larvae | LC₅₀/(μg/mL) | Limit/(μg/mL) |
| Spodoptera exigua | 55.7 | 21.5-247.1 |
| Plutella xylostella | 12.5 | 0.15-16.1 |
| Helicoverpa armigera | 28.1 | 0.27-7.31 |

The results of this analysis indicated that the HD72Aa1 transformant exhibited insecticidal activity against each of Spodoptera exigua, Plutella xylostella and Helicoverpa armigera larvae, whereas the negative controls exhibited no insecticidal activity.

### Example 4

### Isolation of Bt cry72Aa1 Operon and Recombinant Protein Expression

Total DNA from strain HS18-1 was extracted, as described in Example 1, and used as a template for PCR amplification of the cry72Aa1 operon. The PCR reaction included 1.5 μl forward primer 72AF: 5′-ATG TCT AAT CGT TAT CCA CG-3′ (SEQ ID NO: 21), 1.5 μl reverse primer 72AOR: 5′-TTA ACG GCT GTA TCC TTG ATT-3′ (SEQ ID NO: 22), 2 μl template DNA, 7.5 μl double distilled water and 12.5 μl TAQMIX (Biomed Biotech, Beijing, China). The thermocycling reaction included pre-denaturation at 94° C. for 5 minutes; 30 cycles of: denaturation at 94° C. for 50 seconds, annealing at 54° C. for 50 seconds, and extension at 72° C. for 3.5 minutes; and a final extension at 72° C. for 10 minutes. The amplification reaction products were subjected to electrophoresis in 0.7% agarose gel, and placed into a gel imaging system for observing the PCR amplification products. A 3754 bp amplicon containing the Cry72Aa1 operon was obtained. Nucleotide sequence analysis of the operon indicated the presence of the 2064 bp cry72Aa1 gene (SEQ ID NO: 4), a 1623 bp Cry72orf2 gene (SEQ ID NO: 5), and a 67 bp non-coding spacer therebetween. The full-length operon is provided herein as SEQ ID NO: 6.

Sequence analysis indicated that the Cry72orf2 gene of the cry72Aa1 operon encoded a protein of 540 amino acid residues, the sequence of which is provided herein as SEQ ID NO: 10. The amino acid composition of the Cry72Orf2 protein is provided in Table 7.

TABLE 7

| Amino Acid | Number | Percentage | Amino Acid | Number | Percentage |
|---|---|---|---|---|---|
| Ala(A): | 27 | 3.39 | Met(M): | 15 | 3.15 |
| Cys(C): | 11 | 1.88 | Asn(N): | 40 | 7.45 |
| Asp(D): | 38 | 7.13 | Pro(P): | 19 | 3.08 |
| Glu(E): | 32 | 6.64 | Gln(Q): | 31 | 6.38 |
| Phe(F): | 15 | 3.49 | Arg(R): | 18 | 4.42 |
| Gly(G): | 41 | 4.34 | Ser(S): | 33 | 4.89 |
| His(H): | 20 | 3.70 | Thr(T): | 35 | 5.88 |
| Ile(I): | 31 | 5.73 | Val(V): | 27 | 4.46 |
| Lys(K): | 32 | 6.59 | Trp(W): | 6 | 1.73 |
| Leu(L): | 37 | 6.84 | Tyr(Y): | 32 | 8.17 |

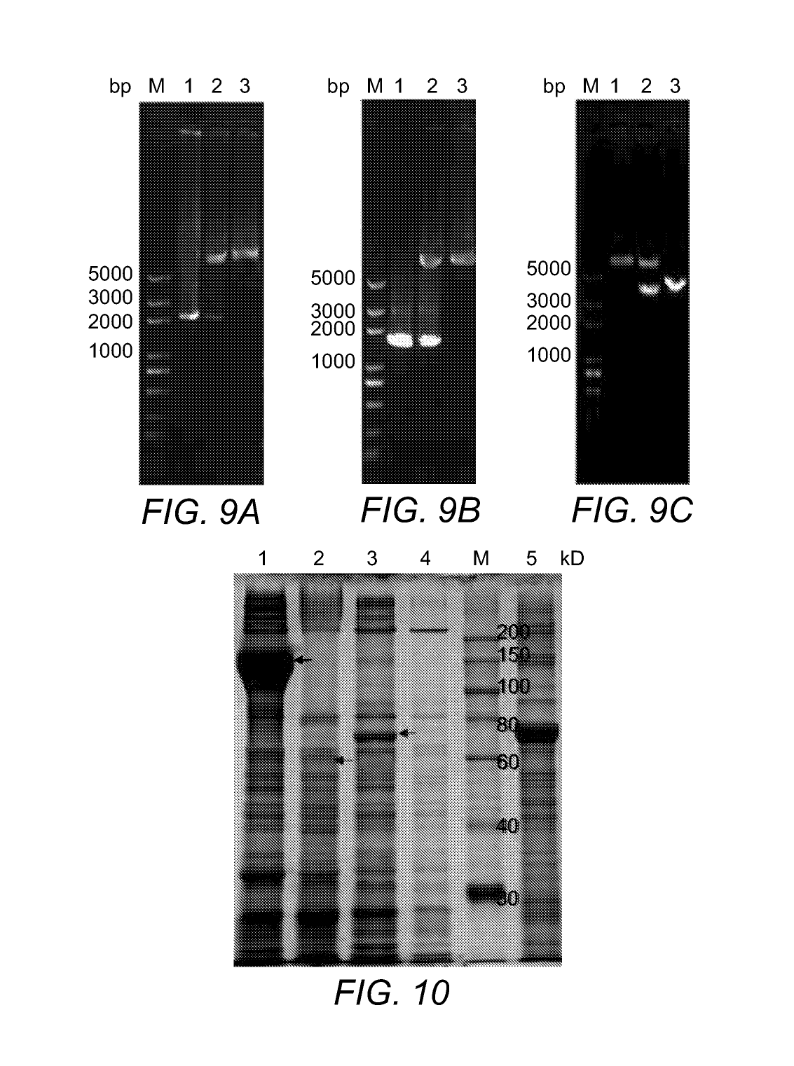

The open reading frames of the cry72Aa1 operon were amplified using gene-specific primers. Cry72Aa1 was amplified with pS72-F (SEQ ID NO: 17) and pS72-R (SEQ ID NO:18), Cry72orf2 was amplified with pS72O-F (5′-GGG GTC GAC AAT GTT TAC AAG TGG CAC GAA A-3′; SEQ ID NO:19) and pS720-R (5′-CCC CTC GAG TTA ACG GCT GTA TCC TTG ATT A-3′; SEQ ID NO: 20), and the complete cry72Aa1 operon was amplified with pS72-F and pS720-R. Amplification was performed using total DNA from strain HS18-1 as a template. The resulting amplicons were digested with SalI and XhoI and inserted into vector pSTK. Recombinant plasmids were transformed and amplified in trans1-T1 competent cells (TransGen Biotech, Inc., China). Plasmids were extracted and subjected to enzyme digestion and electrophoresis to confirm the size of the insert (FIGS. 9A-9C). Plasmids were subsequently demethylated with trans110 competent cells (TransGen Biotech, Inc., China) and transferred into the no-crystal mutant strain HD73⁻ by electroporation (2.2 kV, 1000Ω, 25 μF). The recombinant plasmids were named pSTK-cry72Aa1, pSTK-Cry72orf2 and pSTK-cry72Aa1-Cry72orf2, and the transformants containing the recombinant plasmids were named HD72, HD72orf, and HD720, respectively.

All transformants were cultivated in ½ LB medium at 30° C., 200 r/min for 72 hours. The culture solution was subsequently centrifuged to collect thalli, and the supernatant was discarded. The thalli were washed with sterile water three times; 30 mL 10 mmol/L Tris-HCl (pH 8.0) was added and the cells were disrupted by ultrasonication. Proteins were extracted and detected by SDS-PAGE. As shown in FIG. 10, SDS-PAGE analysis indicated that the HD72O transformant expressed a protein having a molecular weight of about 150 kDa (Cry72Aa1 protein and its chaperone, Cry72Orf2), whereas transformant HD72 only expressed the cry72Aa1 protein (˜77 kDa) and transformant HD72orf only expressed the Cry72Orf2 protein (60 kDa).

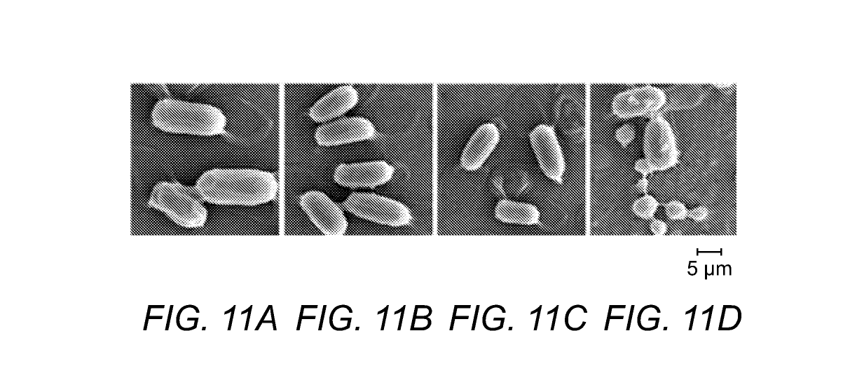

To microscopically observe the expression of the Cry72Aa1 and Cry72Orf2 proteins in HD73⁻ cells, the transformants were cultivated in ½ LB medium at 28° C., 200 r/min, and thalli were collected after more than 90% sporulation. For optical microscopy, a slide was prepared for observing whether the transformants produced a crystal protein. Crystal morphology was also observed using a scanning electron microscope (Hitachi Co., Ltd., Japan). This analysis indicated that the complete Cry72Aa1-Cry72orf2 operon gene was expressed in HD73⁻ and formed spherical parasporal crystals (FIG. 11D), whereas the cry72Aa1 gene alone was expressed in HD73⁻ at low levels (FIG. 11B), and the Cry72orf2 gene alone did not express the parasporal crystals (FIG. 11C).

To demonstrate insecticidal activity against Spodoptera exigua, mixtures of spores and crystals were obtained after cultivating the HD72, HD72orf, and HD720 strains at 28° C., 200 r/min for 72 hours. The mixtures were prepared at five different concentrations with HD72 at 1.5, 2.7, 4.8, 8.7 and 15.8 μg/mL; HD72orf at 0.1, 0.8, 1.6, 3.2, 6.4 and 12 μg/mL; and HD720 at 1.2, 22.7, 7.4, 13.2, 40.6 μg/mL. Subsequently, 45 1-year-old larvae of Spodoptera exigua were contacted with each mixture. Experiments were repeated three times with the pSTK plasmid transformed Bacillus thuringiensis no-crystal mutant strain HD73⁻ as a negative control, and clear water as a blank control. The results were counted after 72 hours, and LC₅₀ was analyzed using SPSS13.0 software. The results of this analysis are presented in Table 8.

TABLE 8

| | | 95% Confidence |
|---|---|---|
| Transformant | LC₅₀/(μg/mL) | Limit/(μg/mL) |
| HD72 | 55.7 | 21.5-247.1 |
| HD72O | 20.8 | 12.9-42.8 |
| HD72orf | N | N |
| Negative Control | N | N |

N represents no insecticidal activity.

The results of this analysis indicated that the expression product of the transformant HD720 exhibited good insecticidal activity against Spodoptera exigua with an LC₅₀ of 20.8 μg/mL. By comparison, the expression product of the transformant HD72 exhibited some insecticidal activity against Spodoptera exigua (LC₅₀ of 210.1 μg/mL), whereas neither the HD72orf transformant nor the negative control exhibited any insecticidal activity against Spodoptera exigua.

The foregoing is illustrative of the present invention and is not to be construed as limiting thereof. Although a few exemplary embodiments of this invention have been described, those skilled in the art will readily appreciate that many modifications are possible in the exemplary embodiments without materially departing from the novel teachings and advantages of this invention. Accordingly, all such modifications are intended to be included within the scope of the present invention.

1. A nonnaturally occurring nucleic acid encoding an insecticidal protein comprising:
(a) the nucleotide sequence set forth in SEQ ID NO: 1;
(b) the nucleotide sequence set forth in SEQ ID NO: 2;
(c) the nucleotide sequence set forth in SEQ ID NO: 3;
(d) the nucleotide sequence set forth in SEQ ID NO: 4;
(e) the nucleotide sequence set forth in SEQ ID NO: 5;
(f) the nucleotide sequence set forth in SEQ ID NO: 6;
(g) a nucleotide sequence that is at least 95% identical to the nucleotide sequence of any one of SEQ ID NOs: 1 to 6;
(h) a nucleotide sequence that encodes a polypeptide comprising the amino acid sequence set forth in SEQ ID NO: 7;
(i) a nucleotide sequence that encodes a polypeptide comprising the amino acid sequence set forth in SEQ ID NO: 8;
(j) a nucleotide sequence that encodes a polypeptide comprising the amino acid sequence set forth in SEQ ID NO: 9;
(k) a nucleotide sequence that encodes a polypeptide comprising the amino acid sequence set forth in SEQ ID NO: 10;
(l) a nucleotide sequence that encodes a polypeptide comprising an amino acid sequence that is at least 95% identical to the amino acid sequence set forth in any one of SEQ ID NOs: 7-10;
(m) a nucleotide sequence that is complementary to the nucleotide sequence of any one of (a) to (l) above;
(n) a nucleotide sequence that hybridizes to the nucleotide sequence of any one of (a) to (m) above under stringent hybridization conditions;
(o) a functional fragment of any one of (a), (c), (d), (f), (g), (h) and (j) above, wherein the functional fragment encodes an insecticidal δ-endotoxin;
(p) a functional fragment of any one of (a), (c), (d), (f), (g), (h) and (j) above, wherein the functional fragment encodes a polypeptide that comprises an N-terminal helical bundle domain, a central beta-sheet domain and a C-terminal beta-sandwich domain; and/or
(q) a functional fragment of any one of (b), (c), (e), (f), (g), (i) and (k) above, wherein the functional fragment encodes a polypeptide the expression of which increases the expression, stability and/or activity of one or more δ-endotoxins; or
(r) a nucleotide sequence of any one of (a) to (q) above that has been codon optimized for expression in a transgenic plant.

2.-5. (canceled)

6. An expression cassette or vector comprising the nonnaturally occurring nucleic acid of claim 1 operably linked to a heterologous promoter.

7. A transgenic bacterium or transgenic plant or plant part comprising the nonnaturally occurring nucleic acid of claim 1.

8.-10. (canceled)

11. The transgenic plant or plant part of claim 7, wherein the plant or plant part is (a) a monocot, optionally rice, maize, wheat, barley, oats, rye, millet, sorghum, buckwheat, or sugar cane or (b) a dicot, optionally cotton, soybean, sugar beet or sunflower.

12.-13. (canceled)

14. A product harvested from the transgenic plant or plant part of claim 11.

15. A processed product produced from the harvested product of claim 14.

16. (canceled)

17. An isolated protein comprising:
(a) an amino acid sequence set forth in any of SEQ ID NOs: 7-10;
(b) an amino acid sequence that is at least 95% identical to the amino acid sequence set forth in any of SEQ ID NOs: 7-10;
(c) a functional fragment of any one of (a) to (b) above, wherein the functional fragment comprises an insecticidal δ-endotoxin; and/or
(d) a functional fragment of any one of (a) to (b) above, wherein the functional fragment comprises an N-terminal helical bundle domain, a central beta-sheet domain and a C-terminal beta-sandwich domain.

18.-21. (canceled)

22. A pesticidal composition comprising the isolated protein of claim 17.

23.-31. (canceled)

32. A method of producing a plant having enhanced pest resistance, comprising:
introducing into a plant the nucleic acid of claim 1, wherein expression of the insecticidal protein results in enhanced pest resistance in the plant as compared to a control plant of the same species grown under the same environmental conditions.

33. The method of claim 32, wherein the nucleic acid is introduced into the plant by transforming the plant.

34. The method of claim 32, wherein the nucleic acid is introduced by crossing a first parent plant comprising the nucleic acid with a different second parent plant,
wherein the progeny generation comprises at least one plant that possesses the exogenous nucleic acid within its genome and that exhibits enhanced pest resistance as compared to a control plant of the same species grown under the same environmental conditions.

35.-44. (canceled)

45. A method of controlling an insect pest, comprising expressing the nucleic acid of claim 1 in a plant.

46. (canceled)

47. The method of claim 45, wherein the plant is a monocot, optionally rice, maize, wheat, barley, oats, rye, millet, sorghum, buckwheat, or sugar cane.

48. (canceled)
